SEQUENCE LISTING (627 sequences)

SEQ ID NO: 1 - RNA, 18 nt, synthetic construct
  source 1..18: mol_type = other RNA
cagaggccagctgactgc

SEQ ID NO: 2 - RNA, 17 nt, synthetic construct
  source 1..17: mol_type = other RNA
tgtactcacagtcagtg

SEQ ID NO: 3 - RNA, 17 nt, synthetic construct
  source 1..17: mol_type = other RNA
aggaggcagcttccctg

SEQ ID NO: 4 - RNA, 16 nt, synthetic construct
  source 1..16: mol_type = other RNA
actgacgcgcggactg

SEQ ID NO: 5 - RNA, 16 nt, synthetic construct
  source 1..16: mol_type = other RNA
ctgcgcgagtctcccg

SEQ ID NO: 6 - RNA, 20 nt, synthetic construct
  source 1..20: mol_type = other RNA
tgccagccagggcgtgcagg

SEQ ID NO: 7 - RNA, 20 nt, synthetic construct
  source 1..20: mol_type = other RNA
cgagaatgcagcggcaacag

SEQ ID NO: 8 - RNA, 20 nt, synthetic construct
  source 1..20: mol_type = other RNA
aacagtccagaaatttctcc

SEQ ID NO: 9 - RNA, 20 nt, synthetic construct
  source 1..20: mol_type = other RNA
ccaagcaatgtgcacgtcac

SEQ ID NO: 10 - RNA, 20 nt, synthetic construct
  source 1..20: mol_type = other RNA
caaagagatggaggcttcca

SEQ ID NO: 11 - RNA, 19 nt, synthetic construct
  source 1..19: mol_type = other RNA
agaggccagctgactgctt

SEQ ID NO: 12 - RNA, 19 nt, synthetic construct
  source 1..19: mol_type = other RNA
acagaggccagctgactgc

SEQ ID NO: 13 - RNA, 17 nt, synthetic construct
  source 1..17: mol_type = other RNA
agaacagaggccagctg

SEQ ID NO: 14 - RNA, 17 nt, synthetic construct
  source 1..17: mol_type = other RNA
gaacagaggccagctga

SEQ ID NO: 15 - RNA, 17 nt, synthetic construct
  source 1..17: mol_type = other RNA
aacagaggccagctgac

SEQ ID NO: 16 - RNA, 17 nt, synthetic construct
  source 1..17: mol_type = other RNA
acagaggccagctgact

SEQ ID NO: 17 - RNA, 17 nt, synthetic construct
  source 1..17: mol_type = other RNA
cagaggccagctgactg

SEQ ID NO: 18 - RNA, 17 nt, synthetic construct
  source 1..17: mol_type = other RNA
agaggccagctgactgc

SEQ ID NO: 19 - RNA, 17 nt, synthetic construct
  source 1..17: mol_type = other RNA
gaggccagctgactgct

SEQ ID NO: 20 - RNA, 19 nt, synthetic construct
  source 1..19: mol_type = other RNA
cagaggccagctgactgct

SEQ ID NO: 21 - RNA, 17 nt, synthetic construct
  source 1..17: mol_type = other RNA
aggccagctgactgctt

SEQ ID NO: 22 - RNA, 18 nt, synthetic construct
  source 1..18: mol_type = other RNA
agaacagaggccagctga

SEQ ID NO: 23 - RNA, 18 nt, synthetic construct
  source 1..18: mol_type = other RNA
gaacagaggccagctgac

SEQ ID NO: 24 - RNA, 18 nt, synthetic construct
  source 1..18: mol_type = other RNA
aacagaggccagctgact

SEQ ID NO: 25 - RNA, 18 nt, synthetic construct
  source 1..18: mol_type = other RNA
acagaggccagctgactg

SEQ ID NO: 26 - RNA, 18 nt, synthetic construct
  source 1..18: mol_type = other RNA
agaggccagctgactgct

SEQ ID NO: 27 - RNA, 18 nt, synthetic construct
  source 1..18: mol_type = other RNA
gaggccagctgactgctt

SEQ ID NO: 28 - RNA, 18 nt, synthetic construct
  source 1..18: mol_type = other RNA
aggccagctgactgcttg

SEQ ID NO: 29 - RNA, 18 nt, synthetic construct
  source 1..18: mol_type = other RNA
ggccagctgactgcttga

SEQ ID NO: 30 - RNA, 17 nt, synthetic construct
  source 1..17: mol_type = other RNA
ggccagctgactgcttg

SEQ ID NO: 31 - RNA, 19 nt, synthetic construct
  source 1..19: mol_type = other RNA
gaacagaggccagctgact

SEQ ID NO: 32 - RNA, 19 nt, synthetic construct
  source 1..19: mol_type = other RNA
gaggccagctgactgcttg

SEQ ID NO: 33 - RNA, 19 nt, synthetic construct
  source 1..19: mol_type = other RNA
aacagaggccagctgactg

SEQ ID NO: 34 - RNA, 17 nt, synthetic construct
  source 1..17: mol_type = other RNA
gccagctgactgcttga

SEQ ID NO: 35 - RNA, 16 nt, synthetic construct
  source 1..16: mol_type = other RNA
actcacagtcagtgaa

SEQ ID NO: 36 - RNA, 16 nt, synthetic construct
  source 1..16: mol_type = other RNA
tactcacagtcagtga

SEQ ID NO: 37 - RNA, 16 nt, synthetic construct
  source 1..16: mol_type = other RNA
gtactcacagtcagtg

SEQ ID NO: 38 - RNA, 16 nt, synthetic construct
  source 1..16: mol_type = other RNA
tgtactcacagtcagt

SEQ ID NO: 39 - RNA, 16 nt, synthetic construct
  source 1..16: mol_type = other RNA
ttgtactcacagtcag

SEQ ID NO: 40 - RNA, 16 nt, synthetic construct
  source 1..16: mol_type = other RNA
gttgtactcacagtca

SEQ ID NO: 41 - RNA, 16 nt, synthetic construct
  source 1..16: mol_type = other RNA
ggttgtactcacagtc

SEQ ID NO: 42 - RNA, 16 nt, synthetic construct
  source 1..16: mol_type = other RNA
tggttgtactcacagt

SEQ ID NO: 43 - RNA, 20 nt, synthetic construct
  source 1..20: mol_type = other RNA
tgtactcacagtcagtgaag

SEQ ID NO: 44 - RNA, 20 nt, synthetic construct
  source 1..20: mol_type = other RNA
ttgtactcacagtcagtgaa

SEQ ID NO: 45 - RNA, 18 nt, synthetic construct
  source 1..18: mol_type = other RNA
gttgtactcacagtcagt

SEQ ID NO: 46 - RNA, 18 nt, synthetic construct
  source 1..18: mol_type = other RNA
ttgtactcacagtcagtg

SEQ ID NO: 47 - RNA, 18 nt, synthetic construct
  source 1..18: mol_type = other RNA
tgtactcacagtcagtga

SEQ ID NO: 48 - RNA, 18 nt, synthetic construct
  source 1..18: mol_type = other RNA
gtactcacagtcagtgaa

SEQ ID NO: 49 - RNA, 18 nt, synthetic construct
  source 1..18: mol_type = other RNA
tactcacagtcagtgaag

SEQ ID NO: 50 - RNA, 18 nt, synthetic construct
  source 1..18: mol_type = other RNA
actcacagtcagtgaaga

SEQ ID NO: 51 - RNA, 20 nt, synthetic construct
  source 1..20: mol_type = other RNA
ggttgtactcacagtcagtg

SEQ ID NO: 52 - RNA, 20 nt, synthetic construct
  source 1..20: mol_type = other RNA
gttgtactcacagtcagtga

SEQ ID NO: 53 - RNA, 16 nt, synthetic construct
  source 1..16: mol_type = other RNA
ctcacagtcagtgaag

SEQ ID NO: 54 - RNA, 18 nt, synthetic construct
  source 1..18: mol_type = other RNA
ggttgtactcacagtcag

SEQ ID NO: 55 - RNA, 18 nt, synthetic construct
  source 1..18: mol_type = other RNA
tggttgtactcacagtca

SEQ ID NO: 56 - RNA, 16 nt, synthetic construct
  source 1..16: mol_type = other RNA
tcacagtcagtgaaga

SEQ ID NO: 57 - RNA, 18 nt, synthetic construct
  source 1..18: mol_type = other RNA
aggaggcagcttccctgc

SEQ ID NO: 58 - RNA, 18 nt, synthetic construct
  source 1..18: mol_type = other RNA
gaggcagcttccctgctc

SEQ ID NO: 59 - RNA, 18 nt, synthetic construct
  source 1..18: mol_type = other RNA
ggaggcagcttccctgct

SEQ ID NO: 60 - RNA, 18 nt, synthetic construct
  source 1..18: mol_type = other RNA
gaggaggcagcttccctg

SEQ ID NO: 61 - RNA, 18 nt, synthetic construct
  source 1..18: mol_type = other RNA
ggaggaggcagcttccct

SEQ ID NO: 62 - RNA, 18 nt, synthetic construct
  source 1..18: mol_type = other RNA
tggaggaggcagcttccc

SEQ ID NO: 63 - RNA, 17 nt, synthetic construct
  source 1..17: mol_type = other RNA
ggcagcttccctgctct

SEQ ID NO: 64 - RNA, 17 nt, synthetic construct
  source 1..17: mol_type = other RNA
aggcagcttccctgctc

SEQ ID NO: 65 - RNA, 17 nt, synthetic construct
  source 1..17: mol_type = other RNA
gaggcagcttccctgct

SEQ ID NO: 66 - RNA, 17 nt, synthetic construct
  source 1..17: mol_type = other RNA
gaggaggcagcttccct

SEQ ID NO: 67 - RNA, 17 nt, synthetic construct
  source 1..17: mol_type = other RNA
ggaggaggcagcttccc

SEQ ID NO: 68 - RNA, 17 nt, synthetic construct
  source 1..17: mol_type = other RNA
tggaggaggcagcttcc

SEQ ID NO: 69 - RNA, 17 nt, synthetic construct
  source 1..17: mol_type = other RNA
ttggaggaggcagcttc

SEQ ID NO: 70 - RNA, 17 nt, synthetic construct
  source 1..17: mol_type = other RNA
ggaggcagcttccctgc

SEQ ID NO: 71 - RNA, 16 nt, synthetic construct
  source 1..16: mol_type = other RNA
ggcagcttccctgctc

SEQ ID NO: 72 - RNA, 16 nt, synthetic construct
  source 1..16: mol_type = other RNA
aggcagcttccctgct

SEQ ID NO: 73 - RNA, 16 nt, synthetic construct
  source 1..16: mol_type = other RNA
gaggcagcttccctgc

SEQ ID NO: 74 - RNA, 16 nt, synthetic construct
  source 1..16: mol_type = other RNA
ggaggcagcttccctg

SEQ ID NO: 75 - RNA, 16 nt, synthetic construct
  source 1..16: mol_type = other RNA
aggaggcagcttccct

SEQ ID NO: 76 - RNA, 16 nt, synthetic construct
  source 1..16: mol_type = other RNA
gaggaggcagcttccc

SEQ ID NO: 77 - RNA, 16 nt, synthetic construct
  source 1..16: mol_type = other RNA
ggaggaggcagcttcc

SEQ ID NO: 78 - RNA, 16 nt, synthetic construct
  source 1..16: mol_type = other RNA
tggaggaggcagcttc

SEQ ID NO: 79 - RNA, 16 nt, synthetic construct
  source 1..16: mol_type = other RNA
ttggaggaggcagctt

SEQ ID NO: 80 - RNA, 16 nt, synthetic construct
  source 1..16: mol_type = other RNA
gcagcttccctgctct

SEQ ID NO: 81 - RNA, 15 nt, synthetic construct
  source 1..15: mol_type = other RNA
acgcgcggactgggc

SEQ ID NO: 82 - RNA, 15 nt, synthetic construct
  source 1..15: mol_type = other RNA
gacgcgcggactggg

SEQ ID NO: 83 - RNA, 15 nt, synthetic construct
  source 1..15: mol_type = other RNA
tgacgcgcggactgg

SEQ ID NO: 84 - RNA, 15 nt, synthetic construct
  source 1..15: mol_type = other RNA
ctgacgcgcggactg

SEQ ID NO: 85 - RNA, 15 nt, synthetic construct
  source 1..15: mol_type = other RNA
actgacgcgcggact

SEQ ID NO: 86 - RNA, 15 nt, synthetic construct
  source 1..15: mol_type = other RNA
ccgactgacgcgcgg

SEQ ID NO: 87 - RNA, 15 nt, synthetic construct
  source 1..15: mol_type = other RNA
cgactgacgcgcgga

SEQ ID NO: 88 - RNA, 16 nt, synthetic construct
  source 1..16: mol_type = other RNA
accgactgacgcgcgg

SEQ ID NO: 89 - RNA, 16 nt, synthetic construct
  source 1..16: mol_type = other RNA
acgcgcggactgggcg

SEQ ID NO: 90 - RNA, 16 nt, synthetic construct
  source 1..16: mol_type = other RNA
ccgactgacgcgcgga

SEQ ID NO: 91 - RNA, 15 nt, synthetic construct
  source 1..15: mol_type = other RNA
gactgacgcgcggac

SEQ ID NO: 92 - RNA, 16 nt, synthetic construct
  source 1..16: mol_type = other RNA
cgactgacgcgcggac

SEQ ID NO: 93 - RNA, 17 nt, synthetic construct
  source 1..17: mol_type = other RNA
tgacgcgcggactgggc

SEQ ID NO: 94 - RNA, 16 nt, synthetic construct
  source 1..16: mol_type = other RNA
ctgacgcgcggactgg

SEQ ID NO: 95 - RNA, 16 nt, synthetic construct
  source 1..16: mol_type = other RNA
tgacgcgcggactggg

SEQ ID NO: 96 - RNA, 16 nt, synthetic construct
  source 1..16: mol_type = other RNA
gacgcgcggactgggc

SEQ ID NO: 97 - RNA, 17 nt, synthetic construct
  source 1..17: mol_type = other RNA
accgactgacgcgcgga

SEQ ID NO: 98 - RNA, 17 nt, synthetic construct
  source 1..17: mol_type = other RNA
ccgactgacgcgcggac

SEQ ID NO: 99 - RNA, 17 nt, synthetic construct
  source 1..17: mol_type = other RNA
cgactgacgcgcggact

SEQ ID NO: 100 - RNA, 17 nt, synthetic construct
  source 1..17: mol_type = other RNA
gactgacgcgcggactg

SEQ ID NO: 101 - RNA, 17 nt, synthetic construct
  source 1..17: mol_type = other RNA
gacgcgcggactgggcg

SEQ ID NO: 102 - RNA, 17 nt, synthetic construct
  source 1..17: mol_type = other RNA
actgacgcgcggactgg

SEQ ID NO: 103 - RNA, 17 nt, synthetic construct
  source 1..17: mol_type = other RNA
ctgacgcgcggactggg

SEQ ID NO: 104 - RNA, 16 nt, synthetic construct
  source 1..16: mol_type = other RNA
gactgacgcgcggact

SEQ ID NO: 105 - RNA, 16 nt, synthetic construct
  source 1..16: mol_type = other RNA
gcgagtctcccggctc

SEQ ID NO: 106 - RNA, 16 nt, synthetic construct
  source 1..16: mol_type = other RNA
gcgctgcgcgagtctc

SEQ ID NO: 107 - RNA, 16 nt, synthetic construct
  source 1..16: mol_type = other RNA
ggcgctgcgcgagtct

SEQ ID NO: 108 - RNA, 20 nt, synthetic construct
  source 1..20: mol_type = other RNA
cgctgcgcgagtctcccggc

SEQ ID NO: 109 - RNA, 16 nt, synthetic construct
  source 1..16: mol_type = other RNA
cgctgcgcgagtctcc

SEQ ID NO: 110 - RNA, 20 nt, synthetic construct
  source 1..20: mol_type = other RNA
gctgcgcgagtctcccggct

SEQ ID NO: 111 - RNA, 16 nt, synthetic construct
  source 1..16: mol_type = other RNA
cgcgagtctcccggct

SEQ ID NO: 112 - RNA, 16 nt, synthetic construct
  source 1..16: mol_type = other RNA
gctgcgcgagtctccc

SEQ ID NO: 113 - RNA, 16 nt, synthetic construct
  source 1..16: mol_type = other RNA
tgcgcgagtctcccgg

SEQ ID NO: 114 - RNA, 16 nt, synthetic construct
  source 1..16: mol_type = other RNA
gcgcgagtctcccggc

SEQ ID NO: 115 - RNA, 20 nt, synthetic construct
  source 1..20: mol_type = other RNA
gcgctgcgcgagtctcccgg

SEQ ID NO: 116 - RNA, 18 nt, synthetic construct
  source 1..18: mol_type = other RNA
gcgctgcgcgagtctccc

SEQ ID NO: 117 - RNA, 18 nt, synthetic construct
  source 1..18: mol_type = other RNA
cgctgcgcgagtctcccg

SEQ ID NO: 118 - RNA, 18 nt, synthetic construct
  source 1..18: mol_type = other RNA
gctgcgcgagtctcccgg

SEQ ID NO: 119 - RNA, 18 nt, synthetic construct
  source 1..18: mol_type = other RNA
ctgcgcgagtctcccggc

SEQ ID NO: 120 - RNA, 18 nt, synthetic construct
  source 1..18: mol_type = other RNA
tgcgcgagtctcccggct

SEQ ID NO: 121 - RNA, 18 nt, synthetic construct
  source 1..18: mol_type = other RNA
ggcgctgcgcgagtctcc

SEQ ID NO: 122 - RNA, 18 nt, synthetic construct
  source 1..18: mol_type = other RNA
gcgcgagtctcccggctc

SEQ ID NO: 123 - RNA, 18 nt, synthetic construct
  source 1..18: mol_type = other RNA
agccagggcgtgcagggc

SEQ ID NO: 124 - RNA, 18 nt, synthetic construct
  source 1..18: mol_type = other RNA
gccagggcgtgcagggct

SEQ ID NO: 125 - RNA, 20 nt, synthetic construct
  source 1..20: mol_type = other RNA
cagtgccagccagggcgtgc

SEQ ID NO: 126 - RNA, 20 nt, synthetic construct
  source 1..20: mol_type = other RNA
agtgccagccagggcgtgca

SEQ ID NO: 127 - RNA, 20 nt, synthetic construct
  source 1..20: mol_type = other RNA
gccagccagggcgtgcaggg

SEQ ID NO: 128 - RNA, 20 nt, synthetic construct
  source 1..20: mol_type = other RNA
ccagccagggcgtgcagggc

SEQ ID NO: 129 - RNA, 20 nt, synthetic construct
  source 1..20: mol_type = other RNA
cagccagggcgtgcagggct

SEQ ID NO: 130 - RNA, 18 nt, synthetic construct
  source 1..18: mol_type = other RNA
cagccagggcgtgcaggg

SEQ ID NO: 131 - RNA, 20 nt, synthetic construct
  source 1..20: mol_type = other RNA
gtgccagccagggcgtgcag

SEQ ID NO: 132 - RNA, 18 nt, synthetic construct
  source 1..18: mol_type = other RNA
ccagccagggcgtgcagg

SEQ ID NO: 133 - RNA, 16 nt, synthetic construct
  source 1..16: mol_type = other RNA
gccagggcgtgcaggg

SEQ ID NO: 134 - RNA, 18 nt, synthetic construct
  source 1..18: mol_type = other RNA
tgccagccagggcgtgca

SEQ ID NO: 135 - RNA, 16 nt, synthetic construct
  source 1..16: mol_type = other RNA
agtgccagccagggcg

SEQ ID NO: 136 - RNA, 18 nt, synthetic construct
  source 1..18: mol_type = other RNA
gccagccagggcgtgcag

SEQ ID NO: 137 - RNA, 16 nt, synthetic construct
  source 1..16: mol_type = other RNA
tgccagccagggcgtg

SEQ ID NO: 138 - RNA, 16 nt, synthetic construct
  source 1..16: mol_type = other RNA
gccagccagggcgtgc

SEQ ID NO: 139 - RNA, 16 nt, synthetic construct
  source 1..16: mol_type = other RNA
ccagccagggcgtgca

SEQ ID NO: 140 - RNA, 16 nt, synthetic construct
  source 1..16: mol_type = other RNA
gtgccagccagggcgt

SEQ ID NO: 141 - RNA, 16 nt, synthetic construct
  source 1..16: mol_type = other RNA
agccagggcgtgcagg

SEQ ID NO: 142 - RNA, 16 nt, synthetic construct
  source 1..16: mol_type = other RNA
ccagggcgtgcagggc

SEQ ID NO: 143 - RNA, 18 nt, synthetic construct
  source 1..18: mol_type = other RNA
cagtgccagccagggcgt

SEQ ID NO: 144 - RNA, 18 nt, synthetic construct
  source 1..18: mol_type = other RNA
agtgccagccagggcgtg

SEQ ID NO: 145 - RNA, 18 nt, synthetic construct
  source 1..18: mol_type = other RNA
gtgccagccagggcgtgc

SEQ ID NO: 146 - RNA, 16 nt, synthetic construct
  source 1..16: mol_type = other RNA
cagccagggcgtgcag

SEQ ID NO: 147 - RNA, 18 nt, synthetic construct
  source 1..18: mol_type = other RNA
gaatgcagcggcaacagt

SEQ ID NO: 148 - RNA, 20 nt, synthetic construct
  source 1..20: mol_type = other RNA
gaatgcagcggcaacagtgc

SEQ ID NO: 149 - RNA, 20 nt, synthetic construct
  source 1..20: mol_type = other RNA
agaatgcagcggcaacagtg

SEQ ID NO: 150 - RNA, 20 nt, synthetic construct
  source 1..20: mol_type = other RNA
gagaatgcagcggcaacagt

SEQ ID NO: 151 - RNA, 20 nt, synthetic construct
  source 1..20: mol_type = other RNA
acgagaatgcagcggcaaca

SEQ ID NO: 152 - RNA, 20 nt, synthetic construct
  source 1..20: mol_type = other RNA
cacgagaatgcagcggcaac

SEQ ID NO: 153 - RNA, 20 nt, synthetic construct
  source 1..20: mol_type = other RNA
acacgagaatgcagcggcaa

SEQ ID NO: 154 - RNA, 18 nt, synthetic construct
  source 1..18: mol_type = other RNA
atgcagcggcaacagtgc

SEQ ID NO: 155 - RNA, 18 nt, synthetic construct
  source 1..18: mol_type = other RNA
aatgcagcggcaacagtg

SEQ ID NO: 156 - RNA, 18 nt, synthetic construct
  source 1..18: mol_type = other RNA
agaatgcagcggcaacag

SEQ ID NO: 157 - RNA, 18 nt, synthetic construct
  source 1..18: mol_type = other RNA
gagaatgcagcggcaaca

SEQ ID NO: 158 - RNA, 18 nt, synthetic construct
  source 1..18: mol_type = other RNA
cgagaatgcagcggcaac

SEQ ID NO: 159 - RNA, 16 nt, synthetic construct
  source 1..16: mol_type = other RNA
cacgagaatgcagcgg

SEQ ID NO: 160 - RNA, 16 nt, synthetic construct
  source 1..16: mol_type = other RNA
acgagaatgcagcggc

SEQ ID NO: 161 - RNA, 16 nt, synthetic construct
  source 1..16: mol_type = other RNA
gagaatgcagcggcaa

SEQ ID NO: 162 - RNA, 16 nt, synthetic construct
  source 1..16: mol_type = other RNA
agaatgcagcggcaac

SEQ ID NO: 163 - RNA, 16 nt, synthetic construct
  source 1..16: mol_type = other RNA
gaatgcagcggcaaca

SEQ ID NO: 164 - RNA, 16 nt, synthetic construct
  source 1..16: mol_type = other RNA
cgagaatgcagcggca

SEQ ID NO: 165 - RNA, 16 nt, synthetic construct
  source 1..16: mol_type = other RNA
atgcagcggcaacagt

SEQ ID NO: 166 - RNA, 16 nt, synthetic construct
  source 1..16: mol_type = other RNA
tgcagcggcaacagtg

SEQ ID NO: 167 - RNA, 18 nt, synthetic construct
  source 1..18: mol_type = other RNA
acacgagaatgcagcggc

SEQ ID NO: 168 - RNA, 18 nt, synthetic construct
  source 1..18: mol_type = other RNA
cacgagaatgcagcggca

SEQ ID NO: 169 - RNA, 18 nt, synthetic construct
  source 1..18: mol_type = other RNA
acgagaatgcagcggcaa

SEQ ID NO: 170 - RNA, 16 nt, synthetic construct
  source 1..16: mol_type = other RNA
aatgcagcggcaacag

SEQ ID NO: 171 - RNA, 18 nt, synthetic construct
  source 1..18: mol_type = other RNA
agtccagaaatttctcct

SEQ ID NO: 172 - RNA, 20 nt, synthetic construct
  source 1..20: mol_type = other RNA
agtccagaaatttctcctgc

SEQ ID NO: 173 - RNA, 20 nt, synthetic construct
  source 1..20: mol_type = other RNA
cagtccagaaatttctcctg

SEQ ID NO: 174 - RNA, 20 nt, synthetic construct
  source 1..20: mol_type = other RNA
taacagtccagaaatttctc

SEQ ID NO: 175 - RNA, 20 nt, synthetic construct
  source 1..20: mol_type = other RNA
ctaacagtccagaaatttct

SEQ ID NO: 176 - RNA, 20 nt, synthetic construct
  source 1..20: mol_type = other RNA
cctaacagtccagaaatttc

SEQ ID NO: 177 - RNA, 18 nt, synthetic construct
  source 1..18: mol_type = other RNA
tccagaaatttctcctgc

SEQ ID NO: 178 - RNA, 18 nt, synthetic construct
  source 1..18: mol_type = other RNA
gtccagaaatttctcctg

SEQ ID NO: 179 - RNA, 18 nt, synthetic construct
  source 1..18: mol_type = other RNA
cagtccagaaatttctcc

SEQ ID NO: 180 - RNA, 20 nt, synthetic construct
  source 1..20: mol_type = other RNA
acagtccagaaatttctcct

SEQ ID NO: 181 - RNA, 18 nt, synthetic construct
  source 1..18: mol_type = other RNA
aacagtccagaaatttct

SEQ ID NO: 182 - RNA, 16 nt, synthetic construct
  source 1..16: mol_type = other RNA
ctaacagtccagaaat

SEQ ID NO: 183 - RNA, 16 nt, synthetic construct
  source 1..16: mol_type = other RNA
taacagtccagaaatt

SEQ ID NO: 184 - RNA, 18 nt, synthetic construct
  source 1..18: mol_type = other RNA
acagtccagaaatttctc

SEQ ID NO: 185 - RNA, 16 nt, synthetic construct
  source 1..16: mol_type = other RNA
aacagtccagaaattt

SEQ ID NO: 186 - RNA, 16 nt, synthetic construct
  source 1..16: mol_type = other RNA
acagtccagaaatttc

SEQ ID NO: 187 - RNA, 16 nt, synthetic construct
  source 1..16: mol_type = other RNA
cagtccagaaatttct

SEQ ID NO: 188 - RNA, 16 nt, synthetic construct
  source 1..16: mol_type = other RNA
ccagaaatttctcctg

SEQ ID NO: 189 - RNA, 16 nt, synthetic construct
  source 1..16: mol_type = other RNA
gtccagaaatttctcc

SEQ ID NO: 190 - RNA, 16 nt, synthetic construct
  source 1..16: mol_type = other RNA
tccagaaatttctcct

SEQ ID NO: 191 - RNA, 18 nt, synthetic construct
  source 1..18: mol_type = other RNA
taacagtccagaaatttc

SEQ ID NO: 192 - RNA, 18 nt, synthetic construct
  source 1..18: mol_type = other RNA
ctaacagtccagaaattt

SEQ ID NO: 193 - RNA, 16 nt, synthetic construct
  source 1..16: mol_type = other RNA
agtccagaaatttctc

SEQ ID NO: 194 - RNA, 18 nt, synthetic construct
  source 1..18: mol_type = other RNA
cctaacagtccagaaatt

SEQ ID NO: 195 - RNA, 20 nt, synthetic construct
  source 1..20: mol_type = other RNA
agcaatgtgcacgtcaccca

SEQ ID NO: 196 - RNA, 20 nt, synthetic construct
  source 1..20: mol_type = other RNA
aagcaatgtgcacgtcaccc

SEQ ID NO: 197 - RNA, 20 nt, synthetic construct
  source 1..20: mol_type = other RNA
caagcaatgtgcacgtcacc

SEQ ID NO: 198 - RNA, 20 nt, synthetic construct
  source 1..20: mol_type = other RNA
acccaagcaatgtgcacgtc

SEQ ID NO: 199 - RNA, 20 nt, synthetic construct
  source 1..20: mol_type = other RNA
tacccaagcaatgtgcacgt

SEQ ID NO: 200 - RNA, 18 nt, synthetic construct
  source 1..18: mol_type = other RNA
caatgtgcacgtcaccca

SEQ ID NO: 201 - RNA, 18 nt, synthetic construct
  source 1..18: mol_type = other RNA
gcaatgtgcacgtcaccc

SEQ ID NO: 202 - RNA, 18 nt, synthetic construct
  source 1..18: mol_type = other RNA
agcaatgtgcacgtcacc

SEQ ID NO: 203 - RNA, 18 nt, synthetic construct
  source 1..18: mol_type = other RNA
aagcaatgtgcacgtcac

SEQ ID NO: 204 - RNA, 20 nt, synthetic construct
  source 1..20: mol_type = other RNA
cccaagcaatgtgcacgtca

SEQ ID NO: 205 - RNA, 18 nt, synthetic construct
  source 1..18: mol_type = other RNA
ccaagcaatgtgcacgtc

SEQ ID NO: 206 - RNA, 18 nt, synthetic construct
  source 1..18: mol_type = other RNA
caagcaatgtgcacgtca

SEQ ID NO: 207 - RNA, 16 nt, synthetic construct
  source 1..16: mol_type = other RNA
cccaagcaatgtgcac

SEQ ID NO: 208 - RNA, 16 nt, synthetic construct
  source 1..16: mol_type = other RNA
ccaagcaatgtgcacg

SEQ ID NO: 209 - RNA, 16 nt, synthetic construct
  source 1..16: mol_type = other RNA
caagcaatgtgcacgt

SEQ ID NO: 210 - RNA, 16 nt, synthetic construct
  source 1..16: mol_type = other RNA
aagcaatgtgcacgtc

SEQ ID NO: 211 - RNA, 16 nt, synthetic construct
  source 1..16: mol_type = other RNA
agcaatgtgcacgtca

SEQ ID NO: 212 - RNA, 16 nt, synthetic construct
  source 1..16: mol_type = other RNA
acccaagcaatgtgca

SEQ ID NO: 213 - RNA, 16 nt, synthetic construct
  source 1..16: mol_type = other RNA
caatgtgcacgtcacc

SEQ ID NO: 214 - RNA, 16 nt, synthetic construct
  source 1..16: mol_type = other RNA
aatgtgcacgtcaccc

SEQ ID NO: 215 - RNA, 18 nt, synthetic construct
  source 1..18: mol_type = other RNA
tacccaagcaatgtgcac

SEQ ID NO: 216 - RNA, 18 nt, synthetic construct
  source 1..18: mol_type = other RNA
acccaagcaatgtgcacg

SEQ ID NO: 217 - RNA, 18 nt, synthetic construct
  source 1..18: mol_type = other RNA
cccaagcaatgtgcacgt

SEQ ID NO: 218 - RNA, 16 nt, synthetic construct
  source 1..16: mol_type = other RNA
gcaatgtgcacgtcac

SEQ ID NO: 219 - RNA, 18 nt, synthetic construct
  source 1..18: mol_type = other RNA
agagatggaggcttccag

SEQ ID NO: 220 - RNA, 20 nt, synthetic construct
  source 1..20: mol_type = other RNA
agagatggaggcttccagcg

SEQ ID NO: 221 - RNA, 20 nt, synthetic construct
  source 1..20: mol_type = other RNA
aagagatggaggcttccagc

SEQ ID NO: 222 - RNA, 20 nt, synthetic construct
  source 1..20: mol_type = other RNA
aaagagatggaggcttccag

SEQ ID NO: 223 - RNA, 20 nt, synthetic construct
  source 1..20: mol_type = other RNA
ccaaagagatggaggcttcc

SEQ ID NO: 224 - RNA, 20 nt, synthetic construct
  source 1..20: mol_type = other RNA
tccaaagagatggaggcttc

SEQ ID NO: 225 - RNA, 20 nt, synthetic construct
  source 1..20: mol_type = other RNA
ttccaaagagatggaggctt

SEQ ID NO: 226 - RNA, 18 nt, synthetic construct
  source 1..18: mol_type = other RNA
agatggaggcttccagcg

SEQ ID NO: 227 - RNA, 18 nt, synthetic construct
  source 1..18: mol_type = other RNA
gagatggaggcttccagc

SEQ ID NO: 228 - RNA, 18 nt, synthetic construct
  source 1..18: mol_type = other RNA
aagagatggaggcttcca

SEQ ID NO: 229 - RNA, 16 nt, synthetic construct
  source 1..16: mol_type = other RNA
gatggaggcttccagc

SEQ ID NO: 230 - RNA, 18 nt, synthetic construct
  source 1..18: mol_type = other RNA
caaagagatggaggcttc

SEQ ID NO: 231 - RNA, 18 nt, synthetic construct
  source 1..18: mol_type = other RNA
ccaaagagatggaggctt

SEQ ID NO: 232 - RNA, 18 nt, synthetic construct
  source 1..18: mol_type = other RNA
tccaaagagatggaggct

SEQ ID NO: 233 - RNA, 18 nt, synthetic construct
  source 1..18: mol_type = other RNA
ttccaaagagatggaggc

SEQ ID NO: 234 - RNA, 16 nt, synthetic construct
  source 1..16: mol_type = other RNA
agatggaggcttccag

SEQ ID NO: 235 - RNA, 16 nt, synthetic construct
  source 1..16: mol_type = other RNA
gagatggaggcttcca

SEQ ID NO: 236 - RNA, 16 nt, synthetic construct
  source 1..16: mol_type = other RNA
agagatggaggcttcc

SEQ ID NO: 237 - RNA, 16 nt, synthetic construct
  source 1..16: mol_type = other RNA
aagagatggaggcttc

SEQ ID NO: 238 - RNA, 16 nt, synthetic construct
  source 1..16: mol_type = other RNA
aaagagatggaggctt

SEQ ID NO: 239 - RNA, 16 nt, synthetic construct
  source 1..16: mol_type = other RNA
caaagagatggaggct

SEQ ID NO: 240 - RNA, 16 nt, synthetic construct
  source 1..16: mol_type = other RNA
ccaaagagatggaggc

SEQ ID NO: 241 - RNA, 18 nt, synthetic construct
  source 1..18: mol_type = other RNA
aaagagatggaggcttcc

SEQ ID NO: 242 - RNA, 16 nt, synthetic construct
  source 1..16: mol_type = other RNA
tccaaagagatggagg

SEQ ID NO: 243 - RNA, 18 nt, synthetic construct
  source 1..18: mol_type = other RNA
ccactagtcctgtttgga

SEQ ID NO: 244 - RNA, 20 nt, synthetic construct
  source 1..20: mol_type = other RNA
tttggaggaggcagcttccc

SEQ ID NO: 245 - RNA, 18 nt, synthetic construct
  source 1..18: mol_type = other RNA
ttgtgcttcagtactttc

SEQ ID NO: 246 - RNA, 20 nt, synthetic construct
  source 1..20: mol_type = other RNA
ttgtgcttcagtactttcaa

SEQ ID NO: 247 - RNA, 18 nt, synthetic construct
  source 1..18: mol_type = other RNA
cagcagcacaaatggtgt

SEQ ID NO: 248 - RNA, 16 nt, synthetic construct
  source 1..16: mol_type = other RNA
gagcgaggctgccccg

SEQ ID NO: 249 - RNA, 16 nt, synthetic construct
  source 1..16: mol_type = other RNA
gccagagcgaggctgc

SEQ ID NO: 250 - RNA, 16 nt, synthetic construct
  source 1..16: mol_type = other RNA
gccagagcgaggctgc

SEQ ID NO: 251 - RNA, 16 nt, synthetic construct
  source 1..16: mol_type = other RNA
ggcgcggcgcgagcca

SEQ ID NO: 252 - RNA, 16 nt, synthetic construct
  source 1..16: mol_type = other RNA
gcggactgggcgcggg

SEQ ID NO: 253 - RNA, 16 nt, synthetic construct
  source 1..16: mol_type = other RNA
acgcgcggactgggcg

SEQ ID NO: 254 - RNA, 16 nt, synthetic construct
  source 1..16: mol_type = other RNA
accgactgacgcgcgg

SEQ ID NO: 255 - RNA, 16 nt, synthetic construct
  source 1..16: mol_type = other RNA
cgcagccgcgctaggg

SEQ ID NO: 256 - RNA, 16 nt, synthetic construct
  source 1..16: mol_type = other RNA
gccccgcagccgcgct

SEQ ID NO: 257 - RNA, 16 nt, synthetic construct
  source 1..16: mol_type = other RNA
ccagccgcagctctcc

SEQ ID NO: 258 - RNA, 16 nt, synthetic construct
  source 1..16: mol_type = other RNA
gcctgcggaccccgcc

SEQ ID NO: 259 - RNA, 16 nt, synthetic construct
  source 1..16: mol_type = other RNA
cgacgcctgcggaccc

SEQ ID NO: 260 - RNA, 16 nt, synthetic construct
  source 1..16: mol_type = other RNA
gcgagtctcccggctc

SEQ ID NO: 261 - RNA, 20 nt, synthetic construct
  source 1..20: mol_type = other RNA
ttaaaaaacacaatgcatta

SEQ ID NO: 262 - RNA, 20 nt, synthetic construct
  source 1..20: mol_type = other RNA
gatcacagcactgtggacac

SEQ ID NO: 263 - RNA, 20 nt, synthetic construct
  source 1..20: mol_type = other RNA
gactggaggacttccctcgg

SEQ ID NO: 264 - RNA, 20 nt, synthetic construct
  source 1..20: mol_type = other RNA
tgtcagggcactgtgactta

SEQ ID NO: 265 - RNA, 20 nt, synthetic construct
  source 1..20: mol_type = other RNA
gcgggagtttgcttctcacc

SEQ ID NO: 266 - RNA, 16 nt, synthetic construct
  source 1..16: mol_type = other RNA
cgcgctgggccagccg

SEQ ID NO: 267 - RNA, 18 nt, synthetic construct
  source 1..18: mol_type = other RNA
gatctcctcgtcccgcga

SEQ ID NO: 268 - RNA, 16 nt, synthetic construct
  source 1..16: mol_type = other RNA
gcagtggctcactcct

SEQ ID NO: 269 - RNA, 16 nt, synthetic construct
  source 1..16: mol_type = other RNA
ttttgagacagtcttg

SEQ ID NO: 270 - RNA, 16 nt, synthetic construct
  source 1..16: mol_type = other RNA
cgggcacggtggctca

SEQ ID NO: 271 - RNA, 21 nt, synthetic construct
  source 1..21: mol_type = other RNA
ccccttcttcttgaccttctt

SEQ ID NO: 272 - RNA, 17 nt, synthetic construct
  source 1..17: mol_type = other RNA
caggaggacagcatcct

SEQ ID NO: 273 - RNA, 17 nt, synthetic construct
  source 1..17: mol_type = other RNA
aaggtttaccttctcgg

SEQ ID NO: 274 - RNA, 22 nt, synthetic construct
  source 1..22: mol_type = other RNA
gcctccaggctggggagcggct

SEQ ID NO: 275 - RNA, 22 nt, synthetic construct
  source 1..22: mol_type = other RNA
gatacacagcctccaggctggg

SEQ ID NO: 276 - RNA, 22 nt, synthetic construct
  source 1..22: mol_type = other RNA
tttaccttctcggatggagtga

SEQ ID NO: 277 - RNA, 22 nt, synthetic construct
  source 1..22: mol_type = other RNA
tcctggattagctgagccagca

SEQ ID NO: 278 - RNA, 22 nt, synthetic construct
  source 1..22: mol_type = other RNA
cgagcttgtcctggattagctg

SEQ ID NO: 279 - RNA, 21 nt, synthetic construct
  source 1..21: mol_type = other RNA
ctaagggattttcacctacct

SEQ ID NO: 280 - RNA, 14 nt, synthetic construct
  source 1..14: mol_type = other RNA
agccgcagctctcc

SEQ ID NO: 281 - RNA, 18 nt, synthetic construct
  source 1..18: mol_type = other RNA
cttcttgaccttctttat

SEQ ID NO: 282 - RNA, 22 nt, synthetic construct
  source 1..22: mol_type = other RNA
tactcaccttccattccccctt

SEQ ID NO: 283 - RNA, 22 nt, synthetic construct
  source 1..22: mol_type = other RNA
aactgtactttctactcacctt

SEQ ID NO: 284 - RNA, 17 nt, synthetic construct
  source 1..17: mol_type = other RNA
cagcaggaggacagcat

SEQ ID NO: 285 - RNA, 20 nt, synthetic construct
  source 1..20: mol_type = other RNA
ctcactcgttatgccctcgg

SEQ ID NO: 286 - RNA, 22 nt, synthetic construct
  source 1..22: mol_type = other RNA
gcacctgcaaggaaagagaaca

SEQ ID NO: 287 - RNA, 17 nt, synthetic construct
  source 1..17: mol_type = other RNA
tagcagtcggcgggtcc

SEQ ID NO: 288 - RNA, 20 nt, synthetic construct
  source 1..20: mol_type = other RNA
cggtatttagcagtcggcgg

SEQ ID NO: 289 - RNA, 21 nt, synthetic construct
  source 1..21: mol_type = other RNA
tactcacagtcagtgaagaag

SEQ ID NO: 290 - RNA, 17 nt, synthetic construct
  source 1..17: mol_type = other RNA
caagccagacttacctc

SEQ ID NO: 291 - RNA, 20 nt, synthetic construct
  source 1..20: mol_type = other RNA
acttacctcccccattgttg

SEQ ID NO: 292 - RNA, 17 nt, synthetic construct
  source 1..17: mol_type = other RNA
ttctctccagtattcat

SEQ ID NO: 293 - RNA, 21 nt, synthetic construct
  source 1..21: mol_type = other RNA
ccgggtgacttccctggaaga

SEQ ID NO: 294 - RNA, 21 nt, synthetic construct
  source 1..21: mol_type = other RNA
tttaccttctctccagtattc

SEQ ID NO: 295 - RNA, 22 nt, synthetic construct
  source 1..22: mol_type = other RNA
ctctccagtattcattctcttg

SEQ ID NO: 296 - RNA, 20 nt, synthetic construct
  source 1..20: mol_type = other RNA
tttggtgtcaagtttgtcct

SEQ ID NO: 297 - RNA, 21 nt, synthetic construct
  source 1..21: mol_type = other RNA
ggtagtgtttggtgtcaagtt

SEQ ID NO: 298 - RNA, 22 nt, synthetic construct
  source 1..22: mol_type = other RNA
acctacctgacggcggtggtgc

SEQ ID NO: 299 - RNA, 18 nt, synthetic construct
  source 1..18: mol_type = other RNA
agagaaagcagggactaa

SEQ ID NO: 300 - RNA, 20 nt, synthetic construct
  source 1..20: mol_type = other RNA
aggcagcttccctgctctta

SEQ ID NO: 301 - RNA, 17 nt, synthetic construct
  source 1..17: mol_type = other RNA
aagacagggtctgtgcc

SEQ ID NO: 302 - RNA, 16 nt, synthetic construct
  source 1..16: mol_type = other RNA
gcctcctcaggtgggc

SEQ ID NO: 303 - RNA, 17 nt, synthetic construct
  source 1..17: mol_type = other RNA
aggagtcatgcaatttt

SEQ ID NO: 304 - RNA, 17 nt, synthetic construct
  source 1..17: mol_type = other RNA
gcaacaccatggacctt

SEQ ID NO: 305 - RNA, 17 nt, synthetic construct
  source 1..17: mol_type = other RNA
ggaagataaaaggaggc

SEQ ID NO: 306 - RNA, 17 nt, synthetic construct
  source 1..17: mol_type = other RNA
agcactggcatgggttt

SEQ ID NO: 307 - RNA, 17 nt, synthetic construct
  source 1..17: mol_type = other RNA
ttactgggcatttgagc

SEQ ID NO: 308 - RNA, 17 nt, synthetic construct
  source 1..17: mol_type = other RNA
gtcctgtttggaggagg

SEQ ID NO: 309 - RNA, 17 nt, synthetic construct
  source 1..17: mol_type = other RNA
tagtcctgtttggagga

SEQ ID NO: 310 - RNA, 18 nt, synthetic construct
  source 1..18: mol_type = other RNA
accttgctatggatgcta

SEQ ID NO: 311 - RNA, 18 nt, synthetic construct
  source 1..18: mol_type = other RNA
accttgctatggatgcta

SEQ ID NO: 312 - RNA, 18 nt, synthetic construct
  source 1..18: mol_type = other RNA
agataaaaggaggcagct

SEQ ID NO: 313 - RNA, 18 nt, synthetic construct
  source 1..18: mol_type = other RNA
aaagcagggactaattta

SEQ ID NO: 314 - RNA, 19 nt, synthetic construct
  source 1..19: mol_type = other RNA
aacaccatggaccttgcta

SEQ ID NO: 315 - RNA, 19 nt, synthetic construct
  source 1..19: mol_type = other RNA
tttgagcactggcatgggt

SEQ ID NO: 316 - RNA, 20 nt, synthetic construct
  source 1..20: mol_type = other RNA
ctgggcatttgagcactggc

SEQ ID NO: 317 - RNA, 20 nt, synthetic construct
  source 1..20: mol_type = other RNA
ctaatttactgggcatttga

SEQ ID NO: 318 - RNA, 21 nt, synthetic construct
  source 1..21: mol_type = other RNA
gcagggactaatttactgggc

SEQ ID NO: 319 - RNA, 21 nt, synthetic construct
  source 1..21: mol_type = other RNA
tgctaacccactagtcctgtt

SEQ ID NO: 320 - RNA, 17 nt, synthetic construct
  source 1..17: mol_type = other RNA
ttggctccatctctctg

SEQ ID NO: 321 - RNA, 17 nt, synthetic construct
  source 1..17: mol_type = other RNA
aaggaaaacacacctca

SEQ ID NO: 322 - RNA, 17 nt, synthetic construct
  source 1..17: mol_type = other RNA
ctaggtcccacagccct

SEQ ID NO: 323 - RNA, 17 nt, synthetic construct
  source 1..17: mol_type = other RNA
tctaggtcccacagccc

SEQ ID NO: 324 - RNA, 17 nt, synthetic construct
  source 1..17: mol_type = other RNA
ttctcagttgtctcagt

SEQ ID NO: 325 - RNA, 18 nt, synthetic construct
  source 1..18: mol_type = other RNA
ttcttaggaaattcagga

SEQ ID NO: 326 - RNA, 18 nt, synthetic construct
  source 1..18: mol_type = other RNA
tggaggaacagtagttct

SEQ ID NO: 327 - RNA, 18 nt, synthetic construct
  source 1..18: mol_type = other RNA
ggctccatctctctggtg

SEQ ID NO: 328 - RNA, 18 nt, synthetic construct
  source 1..18: mol_type = other RNA
gccttcctctaggtccca

SEQ ID NO: 329 - RNA, 18 nt, synthetic construct
  source 1..18: mol_type = other RNA
tgctttgtttcctctttc

SEQ ID NO: 330 - RNA, 19 nt, synthetic construct
  source 1..19: mol_type = other RNA
ggatccacaaatgtctaga

SEQ ID NO: 331 - RNA, 19 nt, synthetic construct
  source 1..19: mol_type = other RNA
aacagtagttcttaggaaa

SEQ ID NO: 332 - RNA, 19 nt, synthetic construct
  source 1..19: mol_type = other RNA
tctgtgccttcctctaggt

SEQ ID NO: 333 - RNA, 19 nt, synthetic construct
  source 1..19: mol_type = other RNA
atgacttctctgctttgtt

SEQ ID NO: 334 - RNA, 20 nt, synthetic construct
  source 1..20: mol_type = other RNA
acctcattggctccatctct

SEQ ID NO: 335 - RNA, 20 nt, synthetic construct
  source 1..20: mol_type = other RNA
gtcttttaaatgacttctct

SEQ ID NO: 336 - RNA, 21 nt, synthetic construct
  source 1..21: mol_type = other RNA
ctctctggtgacagtgtggag

SEQ ID NO: 337 - RNA, 21 nt, synthetic construct
  source 1..21: mol_type = other RNA
aggaaaacacacctcattggc

SEQ ID NO: 338 - RNA, 21 nt, synthetic construct
  source 1..21: mol_type = other RNA
tttccccaagacagggtctgt

SEQ ID NO: 339 - RNA, 21 nt, synthetic construct
  source 1..21: mol_type = other RNA
tcctctttctcagttgtctca

SEQ ID NO: 340 - RNA, 18 nt, synthetic construct
  source 1..18: mol_type = other RNA
tatgtttgtgcttcagta

SEQ ID NO: 341 - RNA, 18 nt, synthetic construct
  source 1..18: mol_type = other RNA
tgatatatgtttgtgctt

SEQ ID NO: 342 - RNA, 20 nt, synthetic construct
  source 1..20: mol_type = other RNA
ttgtgcttcagtactttcaa

SEQ ID NO: 343 - RNA, 20 nt, synthetic construct
  source 1..20: mol_type = other RNA
tatgtttgtgcttcagtact

SEQ ID NO: 344 - RNA, 20 nt, synthetic construct
  source 1..20: mol_type = other RNA
tgatatatgtttgtgcttca

SEQ ID NO: 345 - RNA, 22 nt, synthetic construct
  source 1..22: mol_type = other RNA
ttgtgcttcagtactttcaaac

SEQ ID NO: 346 - RNA, 22 nt, synthetic construct
  source 1..22: mol_type = other RNA
tatgtttgtgcttcagtacttt

SEQ ID NO: 347 - RNA, 22 nt, synthetic construct
  source 1..22: mol_type = other RNA
tgatatatgtttgtgcttcagt

SEQ ID NO: 348 - RNA, 18 nt, synthetic construct
  source 1..18: mol_type = other RNA
aacagcagcagcacaaat

SEQ ID NO: 349 - RNA, 18 nt, synthetic construct
  source 1..18: mol_type = other RNA
acgacaacagcagcagca

SEQ ID NO: 350 - RNA, 20 nt, synthetic construct
  source 1..20: mol_type = other RNA
cagcagcacaaatggtgtgc

SEQ ID NO: 351 - RNA, 20 nt, synthetic construct
  source 1..20: mol_type = other RNA
aacagcagcagcacaaatgg

SEQ ID NO: 352 - RNA, 20 nt, synthetic construct
  source 1..20: mol_type = other RNA
acgacaacagcagcagcaca

SEQ ID NO: 353 - RNA, 22 nt, synthetic construct
  source 1..22: mol_type = other RNA
cagcagcacaaatggtgtgctc

SEQ ID NO: 354 - RNA, 22 nt, synthetic construct
  source 1..22: mol_type = other RNA
aacagcagcagcacaaatggtg

SEQ ID NO: 355 - RNA, 22 nt, synthetic construct
  source 1..22: mol_type = other RNA
acgacaacagcagcagcacaaa

SEQ ID NO: 356 - RNA, 16 nt, synthetic construct
  source 1..16: mol_type = other RNA
gggcggggcaagccgg

SEQ ID NO: 357 - RNA, 16 nt, synthetic construct
  source 1..16: mol_type = other RNA
gcgggggcggggcaag

SEQ ID NO: 358 - RNA, 16 nt, synthetic construct
  source 1..16: mol_type = other RNA
gcgcgcgggggcgggg

SEQ ID NO: 359 - RNA, 16 nt, synthetic construct
  source 1..16: mol_type = other RNA
cggcgcgcgcgggggc

SEQ ID NO: 360 - RNA, 16 nt, synthetic construct
  source 1..16: mol_type = other RNA
ccgccggcgcgcgcgg

SEQ ID NO: 361 - RNA, 16 nt, synthetic construct
  source 1..16: mol_type = other RNA
cccgccgccggcgcgc

SEQ ID NO: 362 - RNA, 16 nt, synthetic construct
  source 1..16: mol_type = other RNA
ctgccccgccgccggc

SEQ ID NO: 363 - RNA, 16 nt, synthetic construct
  source 1..16: mol_type = other RNA
gaggctgccccgccgc

SEQ ID NO: 364 - RNA, 16 nt, synthetic construct
  source 1..16: mol_type = other RNA
gccagagcgaggctgc

SEQ ID NO: 365 - RNA, 16 nt, synthetic construct
  source 1..16: mol_type = other RNA
gcgagccagagcgagg

SEQ ID NO: 366 - RNA, 16 nt, synthetic construct
  source 1..16: mol_type = other RNA
cggcgcgagccagagc

SEQ ID NO: 367 - RNA, 16 nt, synthetic construct
  source 1..16: mol_type = other RNA
ggcgcggcgcgagcca

SEQ ID NO: 368 - RNA, 16 nt, synthetic construct
  source 1..16: mol_type = other RNA
cgggggcgcggcgcga

SEQ ID NO: 369 - RNA, 16 nt, synthetic construct
  source 1..16: mol_type = other RNA
ggcgcgggggcgcggc

SEQ ID NO: 370 - RNA, 16 nt, synthetic construct
  source 1..16: mol_type = other RNA
actgggcgcgggggcg

SEQ ID NO: 371 - RNA, 16 nt, synthetic construct
  source 1..16: mol_type = other RNA
agggaccgactgacgc

SEQ ID NO: 372 - RNA, 16 nt, synthetic construct
  source 1..16: mol_type = other RNA
cgctagggaccgactg

SEQ ID NO: 373 - RNA, 16 nt, synthetic construct
  source 1..16: mol_type = other RNA
gccgcgctagggaccg

SEQ ID NO: 374 - RNA, 16 nt, synthetic construct
  source 1..16: mol_type = other RNA
ctccgccccgcagccg

SEQ ID NO: 375 - RNA, 16 nt, synthetic construct
  source 1..16: mol_type = other RNA
agctctccgccccgca

SEQ ID NO: 376 - RNA, 16 nt, synthetic construct
  source 1..16: mol_type = other RNA
ccgcagctctccgccc

SEQ ID NO: 377 - RNA, 16 nt, synthetic construct
  source 1..16: mol_type = other RNA
tgggccagccgcagct

SEQ ID NO: 378 - RNA, 16 nt, synthetic construct
  source 1..16: mol_type = other RNA
gcgctgggccagccgc

SEQ ID NO: 379 - RNA, 16 nt, synthetic construct
  source 1..16: mol_type = other RNA
gggcgcgctgggccag

SEQ ID NO: 380 - RNA, 16 nt, synthetic construct
  source 1..16: mol_type = other RNA
aggtgggcgcgctggg

SEQ ID NO: 381 - RNA, 16 nt, synthetic construct
  source 1..16: mol_type = other RNA
cctcaggtgggcgcgc

SEQ ID NO: 382 - RNA, 16 nt, synthetic construct
  source 1..16: mol_type = other RNA
cgccgcctcctcaggt

SEQ ID NO: 383 - RNA, 16 nt, synthetic construct
  source 1..16: mol_type = other RNA
accccgccgcctcctc

SEQ ID NO: 384 - RNA, 16 nt, synthetic construct
  source 1..16: mol_type = other RNA
gcggaccccgccgcct

SEQ ID NO: 385 - RNA, 16 nt, synthetic construct
  source 1..16: mol_type = other RNA
cccgcgacgcctgcgg

SEQ ID NO: 386 - RNA, 16 nt, synthetic construct
  source 1..16: mol_type = other RNA
tcgtcccgcgacgcct

SEQ ID NO: 387 - RNA, 16 nt, synthetic construct
  source 1..16: mol_type = other RNA
ctcctcgtcccgcgac

SEQ ID NO: 388 - RNA, 16 nt, synthetic construct
  source 1..16: mol_type = other RNA
cgatctcctcgtcccg

SEQ ID NO: 389 - RNA, 16 nt, synthetic construct
  source 1..16: mol_type = other RNA
gctccgatctcctcgt

SEQ ID NO: 390 - RNA, 16 nt, synthetic construct
  source 1..16: mol_type = other RNA
cccggctccgatctcc

SEQ ID NO: 391 - RNA, 16 nt, synthetic construct
  source 1..16: mol_type = other RNA
gtctcccggctccgat

SEQ ID NO: 392 - RNA, 16 nt, synthetic construct
  source 1..16: mol_type = other RNA
ggcgctgcgcgagtct

SEQ ID NO: 393 - RNA, 16 nt, synthetic construct
  source 1..16: mol_type = other RNA
ccatggcgctgcgcga

SEQ ID NO: 394 - RNA, 16 nt, synthetic construct
  source 1..16: mol_type = other RNA
ggggccatggcgctgc

SEQ ID NO: 395 - RNA, 16 nt, synthetic construct
  source 1..16: mol_type = other RNA
aatgggggccatggcg

SEQ ID NO: 396 - RNA, 20 nt, synthetic construct
  source 1..20: mol_type = other RNA
tttaactgcttatttcttca

SEQ ID NO: 397 - RNA, 20 nt, synthetic construct
  source 1..20: mol_type = other RNA
ttggaggggcgacttatttt

SEQ ID NO: 398 - RNA, 20 nt, synthetic construct
  source 1..20: mol_type = other RNA
ctgtgggatgggggcgtgtt

SEQ ID NO: 399 - RNA, 20 nt, synthetic construct
  source 1..20: mol_type = other RNA
tggtgggaagctgcggagcg

SEQ ID NO: 400 - RNA, 20 nt, synthetic construct
  source 1..20: mol_type = other RNA
aaaggaactgaggcgggcgg

SEQ ID NO: 401 - RNA, 20 nt, synthetic construct
  source 1..20: mol_type = other RNA
gctggggaggcaacagacgc

SEQ ID NO: 402 - RNA, 20 nt, synthetic construct
  source 1..20: mol_type = other RNA
gaagagggcatcactgaaca

SEQ ID NO: 403 - RNA, 20 nt, synthetic construct
  source 1..20: mol_type = other RNA
ttttcttttgtttcaaacaa

SEQ ID NO: 404 - RNA, 20 nt, synthetic construct
  source 1..20: mol_type = other RNA
acatgtataagatactcttt

SEQ ID NO: 405 - RNA, 20 nt, synthetic construct
  source 1..20: mol_type = other RNA
tgagcttctctttttaggat

SEQ ID NO: 406 - RNA, 20 nt, synthetic construct
  source 1..20: mol_type = other RNA
tgctgtgcaccaattgcaca

SEQ ID NO: 407 - RNA, 20 nt, synthetic construct
  source 1..20: mol_type = other RNA
aacagtccagaaatttctcc

SEQ ID NO: 408 - RNA, 20 nt, synthetic construct
  source 1..20: mol_type = other RNA
agaaggcgtccattcatcct

SEQ ID NO: 409 - RNA, 20 nt, synthetic construct
  source 1..20: mol_type = other RNA
acaaatcttaaataacgggg

SEQ ID NO: 410 - RNA, 20 nt, synthetic construct
  source 1..20: mol_type = other RNA
ccagggttatgtacaaggtc

SEQ ID NO: 411 - RNA, 20 nt, synthetic construct
  source 1..20: mol_type = other RNA
aaatttctaccgttccatac

SEQ ID NO: 412 - RNA, 20 nt, synthetic construct
  source 1..20: mol_type = other RNA
acaaggttttaaaaacaccc

SEQ ID NO: 413 - RNA, 20 nt, synthetic construct
  source 1..20: mol_type = other RNA
acaaggttttaaaaacaccc

SEQ ID NO: 414 - RNA, 20 nt, synthetic construct
  source 1..20: mol_type = other RNA
acatctctatgattctcaac

SEQ ID NO: 415 - RNA, 20 nt, synthetic construct
  source 1..20: mol_type = other RNA
gaaatactccaagaacacag

SEQ ID NO: 416 - RNA, 20 nt, synthetic construct
  source 1..20: mol_type = other RNA
agcagattagtcctcagtgt

SEQ ID NO: 417 - RNA, 20 nt, synthetic construct
  source 1..20: mol_type = other RNA
gtagggactggaatgaagat

SEQ ID NO: 418 - RNA, 20 nt, synthetic construct
  source 1..20: mol_type = other RNA
atgagagcaggcactgaggg

SEQ ID NO: 419 - RNA, 20 nt, synthetic construct
  source 1..20: mol_type = other RNA
tcacctcccaggttatttgg

SEQ ID NO: 420 - RNA, 20 nt, synthetic construct
  source 1..20: mol_type = other RNA
ctcctgagatatcctgattg

SEQ ID NO: 421 - RNA, 20 nt, synthetic construct
  source 1..20: mol_type = other RNA
aggtctgttccaccttggac

SEQ ID NO: 422 - RNA, 20 nt, synthetic construct
  source 1..20: mol_type = other RNA
agacgctgggaaaggcaaag

SEQ ID NO: 423 - RNA, 20 nt, synthetic construct
  source 1..20: mol_type = other RNA
gctgcactaccgggggtatg

SEQ ID NO: 424 - RNA, 20 nt, synthetic construct
  source 1..20: mol_type = other RNA
caccccagcctccacccaca

SEQ ID NO: 425 - RNA, 20 nt, synthetic construct
  source 1..20: mol_type = other RNA
ctggcctgacttcgtgcaga

SEQ ID NO: 426 - RNA, 20 nt, synthetic construct
  source 1..20: mol_type = other RNA
acaggctgtggaggaggacg

SEQ ID NO: 427 - RNA, 20 nt, synthetic construct
  source 1..20: mol_type = other RNA
aggctggggagggggcagtg

SEQ ID NO: 428 - RNA, 20 nt, synthetic construct
  source 1..20: mol_type = other RNA
ctccagactaactgtttttc

SEQ ID NO: 429 - RNA, 20 nt, synthetic construct
  source 1..20: mol_type = other RNA
agaagggcctgggccacagg

SEQ ID NO: 430 - RNA, 20 nt, synthetic construct
  source 1..20: mol_type = other RNA
ttgggatgatgcctggggac

SEQ ID NO: 431 - RNA, 20 nt, synthetic construct
  source 1..20: mol_type = other RNA
ggactagggaaaatgagctg

SEQ ID NO: 432 - RNA, 20 nt, synthetic construct
  source 1..20: mol_type = other RNA
cctgacccttgaacgaaggc

SEQ ID NO: 433 - RNA, 20 nt, synthetic construct
  source 1..20: mol_type = other RNA
cccatctgttctggtccatt

SEQ ID NO: 434 - RNA, 20 nt, synthetic construct
  source 1..20: mol_type = other RNA
ctgttcaggggcctccagaa

SEQ ID NO: 435 - RNA, 20 nt, synthetic construct
  source 1..20: mol_type = other RNA
ttctccacagccatagccct

SEQ ID NO: 436 - RNA, 20 nt, synthetic construct
  source 1..20: mol_type = other RNA
agtccaacgggccaagaacc

SEQ ID NO: 437 - RNA, 20 nt, synthetic construct
  source 1..20: mol_type = other RNA
gagggtacagggtctgtgtg

SEQ ID NO: 438 - RNA, 20 nt, synthetic construct
  source 1..20: mol_type = other RNA
tgactgaagatgcttgccga

SEQ ID NO: 439 - RNA, 20 nt, synthetic construct
  source 1..20: mol_type = other RNA
atctgaaactgaggataatc

SEQ ID NO: 440 - RNA, 14 nt, synthetic construct
  source 1..14: mol_type = other RNA
cagccgcagctctc

SEQ ID NO: 441 - RNA, 14 nt, synthetic construct
  source 1..14: mol_type = other RNA
tgggccagccgcag

SEQ ID NO: 442 - RNA, 14 nt, synthetic construct
  source 1..14: mol_type = other RNA
cgcgctgggccagc

SEQ ID NO: 443 - RNA, 16 nt, synthetic construct
  source 1..16: mol_type = other RNA
cagccgcagctctccg

SEQ ID NO: 444 - RNA, 16 nt, synthetic construct
  source 1..16: mol_type = other RNA
gtgggcgcgctgggcc

SEQ ID NO: 445 - RNA, 18 nt, synthetic construct
  source 1..18: mol_type = other RNA
cagccgcagctctccgcc

SEQ ID NO: 446 - RNA, 18 nt, synthetic construct
  source 1..18: mol_type = other RNA
tgggccagccgcagctct

SEQ ID NO: 447 - RNA, 18 nt, synthetic construct
  source 1..18: mol_type = other RNA
cgcgctgggccagccgca

SEQ ID NO: 448 - RNA, 18 nt, synthetic construct
  source 1..18: mol_type = other RNA
cgcgctgggccagccgca

SEQ ID NO: 449 - RNA, 14 nt, synthetic construct
  source 1..14: mol_type = other RNA
cagctctccgcccc

SEQ ID NO: 450 - RNA, 14 nt, synthetic construct
  source 1..14: mol_type = other RNA
gggccagccgcagc

SEQ ID NO: 451 - RNA, 16 nt, synthetic construct
  source 1..16: mol_type = other RNA
cagctctccgccccgc

SEQ ID NO: 452 - RNA, 16 nt, synthetic construct
  source 1..16: mol_type = other RNA
agccgcagctctccgc

SEQ ID NO: 453 - RNA, 16 nt, synthetic construct
  source 1..16: mol_type = other RNA
gggccagccgcagctc

SEQ ID NO: 454 - RNA, 18 nt, synthetic construct
  source 1..18: mol_type = other RNA
cagctctccgccccgcag

SEQ ID NO: 455 - RNA, 18 nt, synthetic construct
  source 1..18: mol_type = other RNA
cagctctccgccccgcag

SEQ ID NO: 456 - RNA, 18 nt, synthetic construct
  source 1..18: mol_type = other RNA
gggccagccgcagctctc

SEQ ID NO: 457 - RNA, 18 nt, synthetic construct
  source 1..18: mol_type = other RNA
gcgctgggccagccgcag

SEQ ID NO: 458 - RNA, 14 nt, synthetic construct
  source 1..14: mol_type = other RNA
cgtcccgcgacgcc

SEQ ID NO: 459 - RNA, 14 nt, synthetic construct
  source 1..14: mol_type = other RNA
ctcctcgtcccgcg

SEQ ID NO: 460 - RNA, 14 nt, synthetic construct
  source 1..14: mol_type = other RNA
ccgatctcctcgtc

SEQ ID NO: 461 - RNA, 16 nt, synthetic construct
  source 1..16: mol_type = other RNA
cgtcccgcgacgcctg

SEQ ID NO: 462 - RNA, 16 nt, synthetic construct
  source 1..16: mol_type = other RNA
ccgatctcctcgtccc

SEQ ID NO: 463 - RNA, 16 nt, synthetic construct
  source 1..16: mol_type = other RNA
cggctccgatctcctc

SEQ ID NO: 464 - RNA, 18 nt, synthetic construct
  source 1..18: mol_type = other RNA
cgtcccgcgacgcctgcg

SEQ ID NO: 465 - RNA, 18 nt, synthetic construct
  source 1..18: mol_type = other RNA
ctcctcgtcccgcgacgc

SEQ ID NO: 466 - RNA, 18 nt, synthetic construct
  source 1..18: mol_type = other RNA
ccgatctcctcgtcccgc

SEQ ID NO: 467 - RNA, 18 nt, synthetic construct
  source 1..18: mol_type = other RNA
cggctccgatctcctcgt

SEQ ID NO: 468 - RNA, 14 nt, synthetic construct
  source 1..14: mol_type = other RNA
gatctcctcgtccc

SEQ ID NO: 469 - RNA, 14 nt, synthetic construct
  source 1..14: mol_type = other RNA
gctccgatctcctc

SEQ ID NO: 470 - RNA, 14 nt, synthetic construct
  source 1..14: mol_type = other RNA
tcccggctccgatc

SEQ ID NO: 471 - RNA, 16 nt, synthetic construct
  source 1..16: mol_type = other RNA
gatctcctcgtcccgc

SEQ ID NO: 472 - RNA, 16 nt, synthetic construct
  source 1..16: mol_type = other RNA
tcccggctccgatctc

SEQ ID NO: 473 - RNA, 20 nt, synthetic construct
  source 1..20: mol_type = other RNA
aacacaaggtattatgaagt

SEQ ID NO: 474 - RNA, 20 nt, synthetic construct
  source 1..20: mol_type = other RNA
gatgatgtatgaccccacac

SEQ ID NO: 475 - RNA, 20 nt, synthetic construct
  source 1..20: mol_type = other RNA
ccatcttctcttacaaacac

SEQ ID NO: 476 - RNA, 20 nt, synthetic construct
  source 1..20: mol_type = other RNA
ttatacagagaataaaatga

SEQ ID NO: 477 - RNA, 20 nt, synthetic construct
  source 1..20: mol_type = other RNA
ttctgctttagagctaagtt

SEQ ID NO: 478 - RNA, 20 nt, synthetic construct
  source 1..20: mol_type = other RNA
cctgcatttgctgctttagt

SEQ ID NO: 479 - RNA, 20 nt, synthetic construct
  source 1..20: mol_type = other RNA
gaggatggcgagacagcctt

SEQ ID NO: 480 - RNA, 20 nt, synthetic construct
  source 1..20: mol_type = other RNA
aatgagagctgctgagtctt

SEQ ID NO: 481 - RNA, 20 nt, synthetic construct
  source 1..20: mol_type = other RNA
tggtgtgctcaccactggag

SEQ ID NO: 482 - RNA, 20 nt, synthetic construct
  source 1..20: mol_type = other RNA
gacaacagcagcagcacaaa

SEQ ID NO: 483 - RNA, 20 nt, synthetic construct
  source 1..20: mol_type = other RNA
ccactgttattatatttcac

SEQ ID NO: 484 - RNA, 20 nt, synthetic construct
  source 1..20: mol_type = other RNA
aggggacatttttgtgactt

SEQ ID NO: 485 - RNA, 20 nt, synthetic construct
  source 1..20: mol_type = other RNA
gggcggcgagggggctgggc

SEQ ID NO: 486 - RNA, 20 nt, synthetic construct
  source 1..20: mol_type = other RNA
catggcctgcaggaggtcaa

SEQ ID NO: 487 - RNA, 20 nt, synthetic construct
  source 1..20: mol_type = other RNA
cactagacaagtaatacaca

SEQ ID NO: 488 - RNA, 20 nt, synthetic construct
  source 1..20: mol_type = other RNA
acagcactttgagaggacat

SEQ ID NO: 489 - RNA, 20 nt, synthetic construct
  source 1..20: mol_type = other RNA
aggtggcgccgagctcgcgt

SEQ ID NO: 490 - RNA, 20 nt, synthetic construct
  source 1..20: mol_type = other RNA
aggctctgaaagggaggcgg

SEQ ID NO: 491 - RNA, 20 nt, synthetic construct
  source 1..20: mol_type = other RNA
gagcagagagggcggggagc

SEQ ID NO: 492 - RNA, 20 nt, synthetic construct
  source 1..20: mol_type = other RNA
agagaacaccacaatgcagc

SEQ ID NO: 493 - RNA, 20 nt, synthetic construct
  source 1..20: mol_type = other RNA
gagatttcaaagccttgaga

SEQ ID NO: 494 - RNA, 20 nt, synthetic construct
  source 1..20: mol_type = other RNA
gactaatctcagtgcaaggg

SEQ ID NO: 495 - RNA, 20 nt, synthetic construct
  source 1..20: mol_type = other RNA
gagacggggagagatctgac

SEQ ID NO: 496 - RNA, 20 nt, synthetic construct
  source 1..20: mol_type = other RNA
aggtcgtataagttgggagg

SEQ ID NO: 497 - RNA, 20 nt, synthetic construct
  source 1..20: mol_type = other RNA
ggttccgtcctaaggaaatc

SEQ ID NO: 498 - RNA, 20 nt, synthetic construct
  source 1..20: mol_type = other RNA
cgcccggcgcaggtgcctgc

SEQ ID NO: 499 - RNA, 20 nt, synthetic construct
  source 1..20: mol_type = other RNA
aacaagcagcgggagtaaga

SEQ ID NO: 500 - RNA, 20 nt, synthetic construct
  source 1..20: mol_type = other RNA
tggtccgagggaggggacag

SEQ ID NO: 501 - RNA, 20 nt, synthetic construct
  source 1..20: mol_type = other RNA
cctgaagcatgagcactgtt

SEQ ID NO: 502 - RNA, 20 nt, synthetic construct
  source 1..20: mol_type = other RNA
acatcttcgacaaacaaggt

SEQ ID NO: 503 - RNA, 20 nt, synthetic construct
  source 1..20: mol_type = other RNA
taacagagaaagggaaacca

SEQ ID NO: 504 - RNA, 20 nt, synthetic construct
  source 1..20: mol_type = other RNA
ataaattatttttatataaa

SEQ ID NO: 505 - RNA, 20 nt, synthetic construct
  source 1..20: mol_type = other RNA
ttttttaaaatatccttttg

SEQ ID NO: 506 - RNA, 20 nt, synthetic construct
  source 1..20: mol_type = other RNA
aagtttcaagacagactagc

SEQ ID NO: 507 - RNA, 20 nt, synthetic construct
  source 1..20: mol_type = other RNA
ctgataattttaaggtaaac

SEQ ID NO: 508 - RNA, 20 nt, synthetic construct
  source 1..20: mol_type = other RNA
tactttcaaacactgagatt

SEQ ID NO: 509 - RNA, 20 nt, synthetic construct
  source 1..20: mol_type = other RNA
gatatatgtttgtgcttcag

SEQ ID NO: 510 - RNA, 20 nt, synthetic construct
  source 1..20: mol_type = other RNA
gtacagaatggtacagagat

SEQ ID NO: 511 - RNA, 20 nt, synthetic construct
  source 1..20: mol_type = other RNA
tattagactcaagtgcttta

SEQ ID NO: 512 - RNA, 20 nt, synthetic construct
  source 1..20: mol_type = other RNA
ggggtgctgatttctttatt

SEQ ID NO: 513 - RNA, 20 nt, synthetic construct
  source 1..20: mol_type = other RNA
agccccctggacaccgggaa

SEQ ID NO: 514 - RNA, 20 nt, synthetic construct
  source 1..20: mol_type = other RNA
tgatttctttatttattaga

SEQ ID NO: 515 - RNA, 20 nt, synthetic construct
  source 1..20: mol_type = other RNA
ggggtgctgatttctttatt

SEQ ID NO: 516 - RNA, 20 nt, synthetic construct
  source 1..20: mol_type = other RNA
aggggtgctgatttctttat

SEQ ID NO: 517 - RNA, 20 nt, synthetic construct
  source 1..20: mol_type = other RNA
cgggaaggggtgctgatttc

SEQ ID NO: 518 - RNA, 20 nt, synthetic construct
  source 1..20: mol_type = other RNA
aattatttttatataaataa

SEQ ID NO: 519 - RNA, 20 nt, synthetic construct
  source 1..20: mol_type = other RNA
tttgataaattatttttata

SEQ ID NO: 520 - RNA, 20 nt, synthetic construct
  source 1..20: mol_type = other RNA
tccttttgataaattatttt

SEQ ID NO: 521 - RNA, 20 nt, synthetic construct
  source 1..20: mol_type = other RNA
aaatatccttttgataaatt

SEQ ID NO: 522 - RNA, 16 nt, synthetic construct
  source 1..16: mol_type = other RNA
gagtctcccggctccg

SEQ ID NO: 523 - RNA, 18 nt, synthetic construct
  source 1..18: mol_type = other RNA
gctccgatctcctcgtcc

SEQ ID NO: 524 - RNA, 18 nt, synthetic construct
  source 1..18: mol_type = other RNA
tcccggctccgatctcct

SEQ ID NO: 525 - RNA, 18 nt, synthetic construct
  source 1..18: mol_type = other RNA
gagtctcccggctccgat

SEQ ID NO: 526 - RNA, 16 nt, synthetic construct
  source 1..16: mol_type = other RNA
ccccttcattctctag

SEQ ID NO: 527 - RNA, 16 nt, synthetic construct
  source 1..16: mol_type = other RNA
tcccaccactttggga

SEQ ID NO: 528 - RNA, 16 nt, synthetic construct
  source 1..16: mol_type = other RNA
actcctgtaatcccac

SEQ ID NO: 529 - RNA, 16 nt, synthetic construct
  source 1..16: mol_type = other RNA
tgtgccaagtgcagtg

SEQ ID NO: 530 - RNA, 16 nt, synthetic construct
  source 1..16: mol_type = other RNA
gtcctgcctcagccac

SEQ ID NO: 531 - RNA, 16 nt, synthetic construct
  source 1..16: mol_type = other RNA
tcaagtgattgtcctg

SEQ ID NO: 532 - RNA, 16 nt, synthetic construct
  source 1..16: mol_type = other RNA
actcccgggttcaagt

SEQ ID NO: 533 - RNA, 16 nt, synthetic construct
  source 1..16: mol_type = other RNA
gcaacctccaactccc

SEQ ID NO: 534 - RNA, 16 nt, synthetic construct
  source 1..16: mol_type = other RNA
gagtgcagtggcacaa

SEQ ID NO: 535 - RNA, 16 nt, synthetic construct
  source 1..16: mol_type = other RNA
cgcccaggctggagtg

SEQ ID NO: 536 - RNA, 16 nt, synthetic construct
  source 1..16: mol_type = other RNA
tcttgctctgtcgccc

SEQ ID NO: 537 - RNA, 16 nt, synthetic construct
  source 1..16: mol_type = other RNA
gtgtgccaagtgcagt

SEQ ID NO: 538 - RNA, 16 nt, synthetic construct
  source 1..16: mol_type = other RNA
gtagagacagggtttc

SEQ ID NO: 539 - RNA, 16 nt, synthetic construct
  source 1..16: mol_type = other RNA
acgcccggctaatttt

SEQ ID NO: 540 - RNA, 16 nt, synthetic construct
  source 1..16: mol_type = other RNA
tgtaatcctagcactt

SEQ ID NO: 541 - RNA, 16 nt, synthetic construct
  source 1..16: mol_type = other RNA
ggctcatgcctgtaat

SEQ ID NO: 542 - RNA, 16 nt, synthetic construct
  source 1..16: mol_type = other RNA
atctgtatcttaaatg

SEQ ID NO: 543 - RNA, 16 nt, synthetic construct
  source 1..16: mol_type = other RNA
cccggcctggatctgt

SEQ ID NO: 544 - RNA, 16 nt, synthetic construct
  source 1..16: mol_type = other RNA
agccaccatgcccggc

SEQ ID NO: 545 - RNA, 16 nt, synthetic construct
  source 1..16: mol_type = other RNA
gagatggagttttact

SEQ ID NO: 546 - RNA, 16 nt, synthetic construct
  source 1..16: mol_type = other RNA
tttattttttgagatg

SEQ ID NO: 547 - RNA, 16 nt, synthetic construct
  source 1..16: mol_type = other RNA
gaagtgaacacaagtt

SEQ ID NO: 548 - RNA, 16 nt, synthetic construct
  source 1..16: mol_type = other RNA
gaaaaagagtgaagtg

SEQ ID NO: 549 - RNA, 16 nt, synthetic construct
  source 1..16: mol_type = other RNA
ctgtggaggaggacgc

SEQ ID NO: 550 - RNA, 16 nt, synthetic construct
  source 1..16: mol_type = other RNA
acgaaggcggactagg

SEQ ID NO: 551 - RNA, 16 nt, synthetic construct
  source 1..16: mol_type = other RNA
tccattcctgaccctt

SEQ ID NO: 552 - RNA, 16 nt, synthetic construct
  source 1..16: mol_type = other RNA
agaacccatctgttct

SEQ ID NO: 553 - RNA, 16 nt, synthetic construct
  source 1..16: mol_type = other RNA
ctctgttcaggggcct

SEQ ID NO: 554 - RNA, 16 nt, synthetic construct
  source 1..16: mol_type = other RNA
gtcctgcctcagccac

SEQ ID NO: 555 - RNA, 16 nt, synthetic construct
  source 1..16: mol_type = other RNA
gacagtcttgctctgt

SEQ ID NO: 556 - RNA, 16 nt, synthetic construct
  source 1..16: mol_type = other RNA
ttctttttgagacagt

SEQ ID NO: 557 - RNA, 16 nt, synthetic construct
  source 1..16: mol_type = other RNA
ctcctgtacttaaggt

SEQ ID NO: 558 - RNA, 16 nt, synthetic construct
  source 1..16: mol_type = other RNA
gttttgagacggagtc

SEQ ID NO: 559 - RNA, 16 nt, synthetic construct
  source 1..16: mol_type = other RNA
ttgtttgtttgttttg

SEQ ID NO: 560 - RNA, 16 nt, synthetic construct
  source 1..16: mol_type = other RNA
gtttgtttgtttgttt

SEQ ID NO: 561 - RNA, 16 nt, synthetic construct
  source 1..16: mol_type = other RNA
gccatttacaggactg

SEQ ID NO: 562 - RNA, 16 nt, synthetic construct
  source 1..16: mol_type = other RNA
agccgggtgtcgtggc

SEQ ID NO: 563 - RNA, 16 nt, synthetic construct
  source 1..16: mol_type = other RNA
acaaaaaattagccgg

SEQ ID NO: 564 - RNA, 16 nt, synthetic construct
  source 1..16: mol_type = other RNA
ccaggtgcagtggctc

SEQ ID NO: 565 - RNA, 16 nt, synthetic construct
  source 1..16: mol_type = other RNA
ggcaccatcacccatg

SEQ ID NO: 566 - RNA, 16 nt, synthetic construct
  source 1..16: mol_type = other RNA
acacccaaagcctggc

SEQ ID NO: 567 - RNA, 16 nt, synthetic construct
  source 1..16: mol_type = other RNA
ggggaagatttacacc

SEQ ID NO: 568 - RNA, 16 nt, synthetic construct
  source 1..16: mol_type = other RNA
ctaaagatgaagggga

SEQ ID NO: 569 - RNA, 16 nt, synthetic construct
  source 1..16: mol_type = other RNA
tcaagtcaggtctaaa

SEQ ID NO: 570 - RNA, 16 nt, synthetic construct
  source 1..16: mol_type = other RNA
gaggatgggggtgcgg

SEQ ID NO: 571 - RNA, 16 nt, synthetic construct
  source 1..16: mol_type = other RNA
taacgggggtgaggat

SEQ ID NO: 572 - RNA, 16 nt, synthetic construct
  source 1..16: mol_type = other RNA
gaggggaaggtaacgg

SEQ ID NO: 573 - RNA, 16 nt, synthetic construct
  source 1..16: mol_type = other RNA
agaggggactgagggg

SEQ ID NO: 574 - RNA, 16 nt, synthetic construct
  source 1..16: mol_type = other RNA
gagggactgcaggaaa

SEQ ID NO: 575 - RNA, 16 nt, synthetic construct
  source 1..16: mol_type = other RNA
tggggaggaggaggga

SEQ ID NO: 576 - RNA, 16 nt, synthetic construct
  source 1..16: mol_type = other RNA
cccaggagggtgggga

SEQ ID NO: 577 - RNA, 16 nt, synthetic construct
  source 1..16: mol_type = other RNA
caaggaataaagacga

SEQ ID NO: 578 - RNA, 16 nt, synthetic construct
  source 1..16: mol_type = other RNA
agtctgggcgctcaag

SEQ ID NO: 579 - RNA, 16 nt, synthetic construct
  source 1..16: mol_type = other RNA
aaaagggagcggagtc

SEQ ID NO: 580 - RNA, 16 nt, synthetic construct
  source 1..16: mol_type = other RNA
agggactgcaggaaaa

SEQ ID NO: 581 - RNA, 16 nt, synthetic construct
  source 1..16: mol_type = other RNA
tagagatggggtttca

SEQ ID NO: 582 - RNA, 16 nt, synthetic construct
  source 1..16: mol_type = other RNA
gtatttttagtagaga

SEQ ID NO: 583 - RNA, 16 nt, synthetic construct
  source 1..16: mol_type = other RNA
attaatttttgtattt

SEQ ID NO: 584 - RNA, 16 nt, synthetic construct
  source 1..16: mol_type = other RNA
accacgcccaattaat

SEQ ID NO: 585 - RNA, 16 nt, synthetic construct
  source 1..16: mol_type = other RNA
agagactgggttttac

SEQ ID NO: 586 - RNA, 16 nt, synthetic construct
  source 1..16: mol_type = other RNA
tatttttagtagagac

SEQ ID NO: 587 - RNA, 16 nt, synthetic construct
  source 1..16: mol_type = other RNA
ctaatttttgtatttt

SEQ ID NO: 588 - RNA, 17 nt, synthetic construct
  source 1..17: mol_type = other RNA
tgaacaaaggtacgtca

SEQ ID NO: 589 - RNA, 17 nt, synthetic construct
  source 1..17: mol_type = other RNA
gccagctgactgcttga

SEQ ID NO: 590 - RNA, 17 nt, synthetic construct
  source 1..17: mol_type = other RNA
tttcttttctgtaataa

SEQ ID NO: 591 - RNA, 17 nt, synthetic construct
  source 1..17: mol_type = other RNA
agagtggaagagtttct

SEQ ID NO: 592 - RNA, 17 nt, synthetic construct
  source 1..17: mol_type = other RNA
gaaataaagaaaaagag

SEQ ID NO: 593 - RNA, 17 nt, synthetic construct
  source 1..17: mol_type = other RNA
ataaactaaaaaggaag

SEQ ID NO: 594 - RNA, 18 nt, synthetic construct
  source 1..18: mol_type = other RNA
ggtacgtcacaggaacat

SEQ ID NO: 595 - RNA, 18 nt, synthetic construct
  source 1..18: mol_type = other RNA
gagtttcttttctgtaat

SEQ ID NO: 596 - RNA, 18 nt, synthetic construct
  source 1..18: mol_type = other RNA
aataaagaaaaagagtga

SEQ ID NO: 597 - RNA, 19 nt, synthetic construct
  source 1..19: mol_type = other RNA
tacattttatgcagccaat

SEQ ID NO: 598 - RNA, 19 nt, synthetic construct
  source 1..19: mol_type = other RNA
ccaacttaggcctacattt

SEQ ID NO: 599 - RNA, 19 nt, synthetic construct
  source 1..19: mol_type = other RNA
agaaagcccttcagagtgg

SEQ ID NO: 600 - RNA, 19 nt, synthetic construct
  source 1..19: mol_type = other RNA
taatcaaagaaagcccttc

SEQ ID NO: 601 - RNA, 19 nt, synthetic construct
  source 1..19: mol_type = other RNA
taaaaaggaagaaataaag

SEQ ID NO: 602 - RNA, 20 nt, synthetic construct
  source 1..20: mol_type = other RNA
aaagaaaaagagtgacggaa

SEQ ID NO: 603 - RNA, 20 nt, synthetic construct
  source 1..20: mol_type = other RNA
aaggaagaaataaagaaaaa

SEQ ID NO: 604 - RNA, 21 nt, synthetic construct
  source 1..21: mol_type = other RNA
tgcagccaatgaggtatgaga

SEQ ID NO: 605 - RNA, 21 nt, synthetic construct
  source 1..21: mol_type = other RNA
cttttctgtaataagctgctt

SEQ ID NO: 606 - RNA, 21 nt, synthetic construct
  source 1..21: mol_type = other RNA
gtggaagagtttcttttctgt

SEQ ID NO: 607 - RNA, 20 nt, synthetic construct
  source 1..20: mol_type = other RNA
cttattattcaccaaaagtt

SEQ ID NO: 608 - RNA, 20 nt, synthetic construct
  source 1..20: mol_type = other RNA
tgctccttattattcaccaa

SEQ ID NO: 609 - RNA, 20 nt, synthetic construct
  source 1..20: mol_type = other RNA
ccttattattcaccaaaagt

SEQ ID NO: 610 - RNA, 20 nt, synthetic construct
  source 1..20: mol_type = other RNA
ctgctccttattattcacca

SEQ ID NO: 611 - RNA, 20 nt, synthetic construct
  source 1..20: mol_type = other RNA
ctccttattattcaccaaaa

SEQ ID NO: 612 - RNA, 20 nt, synthetic construct
  source 1..20: mol_type = other RNA
ttaccatgtgatccagcaat

SEQ ID NO: 613 - RNA, 20 nt, synthetic construct
  source 1..20: mol_type = other RNA
aatcctgctccttattattc

SEQ ID NO: 614 - RNA, 20 nt, synthetic construct
  source 1..20: mol_type = other RNA
ggttgctggaagaatctaaa

SEQ ID NO: 615 - RNA, 20 nt, synthetic construct
  source 1..20: mol_type = other RNA
ttgctggaagaatctaaaat

SEQ ID NO: 616 - RNA, 20 nt, synthetic construct
  source 1..20: mol_type = other RNA
gttgctggaagaatctaaaa

SEQ ID NO: 617 - RNA, 20 nt, synthetic construct
  source 1..20: mol_type = other RNA
aggttgctggaagaatctaa

SEQ ID NO: 618 - RNA, 20 nt, synthetic construct
  source 1..20: mol_type = other RNA
aatcaaaggagataataatg

SEQ ID NO: 619 - RNA, 20 nt, synthetic construct
  source 1..20: mol_type = other RNA
caggttgctggaagaatcta

SEQ ID NO: 620 - RNA, 20 nt, synthetic construct
  source 1..20: mol_type = other RNA
gctggaagaatctaaaatca

SEQ ID NO: 621 - RNA, 20 nt, synthetic construct
  source 1..20: mol_type = other RNA
caaattaaaacaacaatacc

SEQ ID NO: 622 - RNA, 20 nt, synthetic construct
  source 1..20: mol_type = other RNA
gttggacaggctggtctcaa

SEQ ID NO: 623 - RNA, 20 nt, synthetic construct
  source 1..20: mol_type = other RNA
gacaaaaacaacaacaaaga

SEQ ID NO: 624 - RNA, 20 nt, synthetic construct
  source 1..20: mol_type = other RNA
gtaggagtttccatttttct

SEQ ID NO: 625 - RNA, 20 nt, synthetic construct
  source 1..20: mol_type = other RNA
ttccacttggggaacctatg

SEQ ID NO: 626 - RNA, 20 nt, synthetic construct
  source 1..20: mol_type = other RNA
agtttccatttttctacctc

SEQ ID NO: 627 - RNA, 20 nt, synthetic construct
  source 1..20: mol_type = other RNA
tggtggatcagttaagcatg

(19) United States
(12) Patent Application Publication
(10) Pub. No.: US 2026/0286357 A1
(43) Pub. Date: Sep. 24, 2026

(54) COMPOSITIONS AND METHODS FOR TREATING STXBP1 DISORDERS

(71) Applicants: BIOMARIN PHARMACEUTICAL INC., Novato, CA (US); DEEP GENOMICS INCORPORATED, Toronto (CA)

(72) Inventors: Jan Hsi LUI, San Francisco, CA (US); Jonathan LEBOWITZ, Novato, CA (US); Guoying Karen YU, Kensington, CA (US); Xing TANG, Richmond, CA (US); Arthur WUSTER, San Anselmo, CA (US); Yumaine CHONG, Toronto (CA); Erle Marie HOLGERSEN, Toronto (CA); Mark George Ford SUN, Toronto (CA); Janine Jia-Ying TRUONG, Toronto (CA)

(21) Appl. No.: 19/477,926

(22) PCT Filed: Apr. 23, 2024
(86) PCT No.: PCT/US2024/025787

§ 371 Date: Oct. 23, 2025

Related U.S. Application Data
(60) Provisional application No. 63/497,836, filed on Apr. 24, 2023.

(51) Int. Cl. C12N 15/113; A61K 31/712; A61K 31/7125

(52) U.S. Cl. CPC C12N 15/113; A61K 31/712; A61K 31/7125; C12N 2310/11; C12N 2310/315; C12N 2310/321; C12N 2310/3513; C12N 2320/33

(57) ABSTRACT

Provided herein are antisense oligonucleotides (ASOs) and pharmaceutically acceptable derivatives thereof that modulate expression of STXBP1 and have activity in treating STXBP1 disorders. Also provided are pharmaceutical compositions containing the ASOs and methods of using the ASOs for treating a subject with an STXBP1 disorder.

### RELATED APPLICATION

This application claims priority to U.S. provisional application No. 63/497,836, filed Apr. 24, 2023. The contents of the above-referenced application are incorporated by reference herein in their entirety.

### FIELD

Provided herein are antisense oligonucleotides (“ASOs”), compositions comprising ASOs, and methods of using the ASOs and compositions for treating, preventing, or delaying the onset of an STXBP1 disorder or a disorder in which disease processes lower STXBP1 levels.

### INCORPORATION BY REFERENCE

All of the patents, patent applications and publications referred to herein are incorporated by reference herein in their entireties. Citation or identification of any reference in this application is not an admission that such reference is available as prior art to this application.

### SEQUENCE LISTING

This application contains a Sequence Listing, which is being submitted herewith as an XML file named “0100STX01WO_SL.xml”, created on Mar. 20, 2024, size 1,103,905 bytes, which is incorporated by reference herein in its entirety.

### BACKGROUND

Syntaxin binding protein 1 (“STXBP1”) is encoded by the STXBP1 gene (also referred to as Munc 18-1) and is involved in synaptic vesicle fusion with the neuronal cell membrane via its multiple interactions with the soluble N-ethylmaleimide sensitive factor adaptor protein receptors (SNARE) complex, and thus vital for neurotransmitter release (Rizo J et al. Annu. Rev. Biophys. 2015, 44, 339-67; Misura K M, et al Nature 2000, 404, 355-62; Dulubova I, et al PNAS 2007, 104, 2697-702; Shen J, et al Cell 2006, 128, 183-95).

The STXBP1 gene is located on chromosome 9q34.1. Its association with disease was discovered in 2008 in studies of patients with Ohtahara Syndrome, a severe early onset epilepsy. In these studies, five patients with Ohtahara syndrome, a severe, early-onset epilepsy characterized by a suppression-burst pattern on EEG and severe psychomotor retardation, were described with a variety of mutations in the STXBP1 gene, including missense, frameshift, splice site, and nonsense mutations (Saitsu H, et al Nat Genet. 2008, 40(6), 782-8). Soon after, patients presenting with other early-onset epileptic encephalopathies including West syndrome, Lennox-Gastaut syndrome, and Dravet syndrome were identified with STXBP1 mutations (Otsuka M, et al Epilepsia 2010, 51, 2449-52; Carvill G L, et al Neurology 2014, 82, 1245-53; Allen A S, et al Nature 2013, 501, 217-21; Grone B P, et al PLoS ONE 2016, 11, e0151148, doi:10.1371/journal.pone.0151148). Since then, a variety of de novo mutations in the STXBP1 gene have been found in individuals resulting in severe encephalopathies with a diverse set of phenotypes (Deprez L, et al Neurology 2010, 75, 1159-65; Hamdan F F, et al Eur. J. Hum. Genet. 2011, 19, 607-9; Stamberger H, et al Neurology 2016, 86, 954-62; Xian J, et al Brain. 2021, awab327, https://doi.org/10.1093/brain/awab327). The majority of the mutations in STXBP1 are spread across the STXBP1 gene, but there are a few mutational hotspots. For example p.Arg406His is the most common recurrent mutation. Missense mutations occur throughout the protein structure with a couple of minor hotspots (Xian J, et al, supra; Abramov D, et al J. Neurochem. 2021, 157, 165-78) and most missense mutations studied to date cause protein destabilization, aggregation, and degradation (Abramov D, et al, supra). Multiple mutations result in the formation of premature termination codons, which have been found early in the protein sequence (L36X) as well as quite late (W522X) (Stamberger H, et al, supra). Disease-causing mutations associated with STXBP1 include missense, nonsense, frameshift, and splice-site mutations, as well as intragenic, whole gene, and multi-gene deletions.

A recent estimate of the incidence for STXBP1-related disorders is approximately 1:30,000 births (Lopez-Rivera J A, et al Brain 2020, 143, 1099-1105) and STXBP1 is the fifth most implicated gene associated with epileptic developmental disorders (Symonds J D et al Eur. J. Paediatr. Neurol. 2020, 24, 15-23), suggesting that these disorders are not as rare as once believed.

Individuals having an STXBP1 disorder display the following symptoms in varying severities: epilepsy, global delay, cognitive impairment (mild to profound), movement disorders, hypotonia and autism. STXBP1 changes are typically new in families and a single copy of a damaged gene is enough to cause the disorder. Virtually all STXBP1 patients demonstrate developmental delay and intellectual disability, the majority of whom can be classified as severe to profound. Approximately 85%-90% of patients experience some form of epilepsy, which usually manifests within the first year. Over 30 types of seizures have been reported in individuals with the most common being focal-onset seizures, generalized-onset seizures, and epileptic spasms (Xian J, et al, supra). About 90% of patients have motor deficits such as dystonia, spasticity, ataxia, hypotonia, and tremor and behavioral issues including hyperactivity, anxiety, aggressiveness, and autism are common occurrences in subsets of patients. The variety of clinical disease phenotypes in the STXBP1 patient population is illustrated in a recent analysis of 534 STXBP1 patients (Xian J, et al, supra). The number of Human Phenotype Ontology (HPO) assigned terms used to describe their clinical features revealed 592 unique terms, with the median number of HPO terms used per patient being 10 (range 1-53); the most common assigned initial HPO terms were ‘Global developmental delay’, ‘Absent speech’, and ‘Infantile spasms.’

The primary mechanism of disorder formation is haploinsufficiency, as approximately 50%-60% of the reported mutations are either deletions, nonsense, frameshift, or splice site variants (Stamberger H, et al, supra; Xian J, et al, supra). A subset of missense mutations studied appear to promote aggregation and may decrease wild type protein levels, thus potentially producing a dominant-negative effect (Guiberson NGL, et al Nature Comm. 2018, 9, 3986-4009). In animal models, haploinsufficiency has been demonstrated to recapitulate several patient phenotypes (Kovacevik J, et al Brain 2018, 141, 1350-1374; Chen W, et al eLife 2020, 9, e48705 DOI: 10.7554/eLife.48705). Genotype-Phenotype relationships have been difficult to establish. In the recent analysis of 534 STXBP1 patients (Xian J, et al, supra), five genetic hotspots with recurrent variants were identified in more than 10 individuals but none were associated with a specific phenotypic feature, though there were several nominal associations.

Thus, there is a need for treatment of STXBP1 disorders, including epilepsy, global delay, cognitive impairment (mild to profound), movement disorders, hypotonia and autism.

### SUMMARY

Provided herein are ASOs, compositions containing the ASOs, and methods of treating or delaying an STXBP1 disorder using the ASOs. As demonstrated in the Examples, the ASOs provided herein are useful for modulating expression of STXBP1. In one embodiment, the ASOs are useful for increasing expression of STXBP1 in a cell such as a neuronal cell. In one embodiment, the ASOs hybridize with, bind to, or target an STXBP1 mRNA or pre-mRNA. In another embodiment, the ASOs modulate splicing of a STXBP1 pre-mRNA. In another embodiment, the ASOs induce exon skipping in a STXBP1 pre-mRNA. In another embodiment, the ASOs hybridize with, bind to, or target a 5′- or 3′-untranslated region (“UTR”) in a STXBP1 mRNA or pre-mRNA. In one embodiment, the STXBP1 mRNA or pre-mRNA is a wild type STXBP1 mRNA or pre-mRNA. In another embodiment, the STXBP1 mRNA or pre-mRNA is a mutant STXBP1 mRNA or pre-mRNA, such as a mutant STXBP1 mRNA or pre-mRNA associated with an STXBP1 disorder.

In one embodiment, the ASOs provided herein are complementary to an STXBP1 mRNA or pre-mRNA. In another embodiment, the ASOs provided herein are reverse complementary to an STXBP1 mRNA or pre-mRNA.

In another embodiment, provided herein is a method of treating or delaying the onset of an STXBP1 disorder by administering to subject having an STXBP1 disorder an ASO provided herein. In certain embodiments, the STXBP1 disorder is epilepsy, global delay, cognitive impairment (mild to profound), a movement disorder, hypotonia or autism. In one embodiment, the subject having an STXBP1 disorder is characterized by STXBP1 haploinsufficiency.

### BRIEF DESCRIPTION OF THE DRAWINGS

FIGS. 1A-G show 6-point dose-response curves generated using STXBP1 Het iNeurons treated by gymnosis with ASOs provided herein. FIG. 1A: SEQ ID NO: 2; FIG. 1B: SEQ ID NO: 17; FIG. 1C: SEQ ID NO: 75; FIG. 1D: SEQ ID NO: 168; FIG. 1E: SEQ ID NO: 197; FIG. 1F: SEQ ID NO: 188; FIG. 1G: SEQ ID NO: 225.

FIGS. 2A-G show the effect of treatment with ASOs provided herein and controls for 17-days at a 5 μM dose on cell viability determined using a Cell-Titer Fluor Assay. FIG. 2A: SEQ ID NO: 2; FIG. 2B: SEQ ID NO: 75; FIG. 2C: SEQ ID NO: 17; FIG. 2D: SEQ ID NO: 168; FIG. 2E: SEQ ID NO: 188; FIG. 2F: SEQ ID NO: 197; FIG. 2G: SEQ ID NO: 225

FIG. 3 shows the immunostimulatory effects on cytokine production following treatment of PBMCs for 24 hours at 10 μM with ASOs provided herein and controls.

FIGS. 4A-J show the results from orthogonal validation of ASOs provided herein and controls using ELISA using STXBP1 Het iNeurons along with each ASO's NHP mismatches.

FIG. 5 shows the results from an ELISA screen at 5 μM using non-HiBiT tagged STXBP1 Het iNeurons for ASOs provided herein and controls.

FIG. 6 shows the level of synaptically-localized STXBP1 protein in STXBP1+/−(HZ) Ngn2-induced neuron cultures, cultured without glia, after treatment with ASOs provided herein and controls.

FIG. 7 shows the level of synaptically-localized STXBP1 protein in WT Ngn2-induced neuron cultures after treatment with ASOs provided herein and controls.

FIG. 8 shows the level of synaptically-localized STXBP1 protein in STXBP1+/−(HZ) Ngn2-induced neuron cultures, cultured with rat glia, after treatment with ASOs provided herein and controls.

FIG. 9 shows the level of bulk STXBP1 protein in Ngn2-induced neuron cultures, cultured without glia, after treatment with ASOs provided herein and controls.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of an ASO or physiological responses that result upon in vivo administration of an ASO, composition or other mixture. Biological activity, thus, encompasses therapeutic effects and pharmacokinetic behavior of such ASOs, compositions and mixtures. Biological activities can be observed in in vitro systems designed to test for such activities. For example, in one embodiment the biological activity of the ASO refers to modulating gene expression. Methods for detecting and/or quantifying changes in gene expression are known in the art, including but not limited to the methods disclosed in the Examples.

As used herein, when the word “oligonucleotide” is used it may be replaced by “antisense oligonucleotide” and vice versa unless otherwise indicated.

As used herein, the term “complementary” encompasses both forward complementary and reverse complementary sequences, as will be apparent to a skilled person from the context. When an oligonucleotide is complementary, it is understood that it can also be reverse complementary. As such, when “an oligonucleotide is complementary to” a target sequence is used, this means that said oligonucleotide is reverse complementary to said target sequence as the sequence of the oligonucleotide is the reverse complement of the target sequence, unless otherwise stated. When “an antisense oligonucleotide is complementary to” a target sequence is used, this means that said antisense oligonucleotide is complementary to said target sequence as the sequence of the antisense oligonucleotide is the reverse complement of the target sequence, unless otherwise stated.

As used herein, the term “(reverse) complementarity” means a stretch of nucleic acids that can hybridize to another stretch of nucleic acids under physiological conditions. An antisense strand is generally said to be complementary to a matching sense strand. In this context, an antisense oligonucleotide is complementary to its target. Hybridization conditions are defined herein. It is thus not absolutely required that all the bases in the region of complementarity are capable of pairing with bases in the opposing strand. For instance, when designing an antisense oligonucleotide, one may want to incorporate for instance a residue that does not base pair with the base on the complementary strand. Mismatches may to some extent be allowed, if under the circumstances in the cell, the stretch of nucleotides is capable of hybridizing to the complementary part. In one embodiment, the ASOs provided herein have 1 or 2 mismatches with a target sequence, such as a target sequence within a STXBP1 mRNA or pre-mRNA.

As used herein, unless mentioned otherwise, the term “binds to” can be replaced with “complementary to”, “hybridizes to”, “overlaps with” and/or “targets” when used in the context of an antisense oligonucleotide which is complementary to a part of a pre-mRNA as identified herein. In this disclosure, such terms are synonymous. As used herein, “hybridizes” is used under physiological conditions in a cell. In one embodiment, the cell is a neuronal cell unless otherwise indicated.

In one embodiment, the ASOs provided herein may have one or more substitutions or modifications relative to ASOs made up of unmodified single stranded DNA or RNA oligonucleotides. Generally, a substitution replaces one chemical group, which might be hydrogen, by another chemical group. When considering the carbon skeleton of organic molecules, an RNA monomer is inherently 2′-substituted because it has a hydroxyl group at its 2′-position. A DNA monomer would therefore not be 2′-substituted, and an RNA monomer can be seen as a 2′-substituted DNA monomer. When an RNA monomer in turn is 2′-substituted, this substitution can have replaced either the 2′-OH or the 2′-H. When an RNA monomer is 2-O-substituted, this substitution replaces the H of the 2′-OH moiety. As a non-limiting example, 2′-O-methyl RNA is a 2′-substituted monomer (-OMe substitutes —H) and a 2′-substituted RNA monomer (-OMe substitutes —OH) and a 2′-O-substituted RNA monomer (-Me substitutes —H), while 2′-F RNA is a 2′-substituted RNA monomer (—F substitutes —OH or —H) yet not a 2′-O-substituted RNA monomer (2′-0 is either no longer present, or is not substituted). 2′-F RNA where F is substituted for 2′-OH is a 2′-F-2′-deoxy RNA, which is also a 2′-F DNA.

As will be understood by a skilled person, throughout this application, the terms “bicyclic nucleic acid”, “BNA”, “BNA scaffold”, “BNA nucleotide”, “BNA nucleoside”, “BNA modification”, or “BNA scaffold modification” may be replaced by conformationally restricted scaffold modification, locked scaffold modification, locked nucleotide, locked nucleoside, locked monomer, or Tm enhancing scaffold modification, or high-affinity modification and the like, as appropriate.

As used herein, “sequence identity” means a relationship between two or more nucleic acid (polynucleotide or nucleotide or oligonucleotide) sequences, as determined by comparing the sequences. In one embodiment, sequence identity is calculated based on the full length of two given SEQ ID NO or on part thereof. Part thereof means at least 50%, 60%, 70%, 80%, 90%, or 100% of both SEQ ID NO. As used herein, “identity” also means the degree of sequence relatedness between nucleic acid sequences, as the case may be, as determined by the match between strings of such sequences.

Methods to determine identity are designed to give the largest match between the sequences tested. Methods to determine identity and similarity are codified in publicly available computer programs. Computer program methods to determine identity and similarity between two sequences include, e.g., the GCG program package (Devereux, J., et al., Nucleic Acids Research 12 (1): 387 (1984)), BestFit, BLASTN, and FASTA (Altschul, S. F. et al., J. Mol. Biol. 215:403-410 (1990)). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990)). The well-known Smith Waterman algorithm may also be used to determine identity.

Parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis.

Hybridization conditions for a nucleic acid molecule may have low or medium or high stringency (southern blotting procedures). Low or medium or high stringency conditions means pre-hybridization and hybridization at 42° C. in 5×SSPE, 0.3% SDS, 200 pg/ml sheared and denatured salmon sperm DNA, and either 25% or 35% or 50% formamide for low or medium or high stringencies respectively. Subsequently, the hybridization reaction is washed three times for 30 minutes each using 2×SSC, 0.2% SDS and either 55° C. or 65° C., or 75° C. for low or medium or high stringencies respectively.

As used herein, treatment means any manner in which one or more of the symptoms of a disease or disorder are ameliorated or otherwise beneficially altered. Treatment also encompasses any pharmaceutical use of the compositions herein, such as use for treating an STXBP1 disorder.

As used herein, amelioration of the symptoms of a particular disorder by administration of a particular ASO or pharmaceutical composition refers to any lessening, whether permanent or temporary, lasting or transient that can be attributed to or associated with administration of the ASO or pharmaceutical composition.

Certain ASOs provided herein possess asymmetric atoms (optical centers) or double bonds; the racemates, diastereomers, tautomers, geometric isomers and individual isomers are encompassed within the scope of the present disclosure. The ASOs provided herein do not include those which are known in the art to be too unstable to synthesize and/or isolate.

The ASOs provided herein may also contain unnatural proportions of atomic isotopes at one or more of the atoms that constitute such ASOs. For example, the ASOs may be radiolabeled with radioactive isotopes, such as for example tritium (³H), iodine-125 (¹²⁵I) or carbon-14 (¹⁴C). All isotopic variations of the ASOs provided herein, whether radioactive or not, are encompassed within the scope of the present disclosure.

As used herein, an STXBP1 gene is well known in the art. In one embodiment, the coding strand of the human STXBP1 gene spans ~80,423 bp and is set forth in NCBI Accession No. NC_000009.12. Mutant STXBP1 genes are also well known in the art. The STXBP1 gene encodes two major splice variants: i) NM_003165 (isoform a), in which 19 consecutive exons are translated to generate a 68.7 kDa protein of 603 amino acids; and ii) NM_001032221 (isoform b), in which exon 19 is skipped, and a stop codon in exon 20 terminates translation to generate a 67.6 kDa protein of 594 amino acids.

### II. Antisense Oligonucleotides for Use in Compositions and Methods

In one embodiment, provided are antisense oligonucleotides (ASOs) that target or bind to a STXBP1 mRNA or pre-mRNA. In one embodiment, the ASOs provided herein are single stranded oligonucleotides. In one embodiment, the ASOs provided herein contain fewer than 50 nucleotides. In one embodiment, the ASOs provided herein comprise a base sequence that is complementary to a target sequence in an STXBP1 mRNA or pre-mRNA. In one embodiment, the ASOs provided herein target or bind to a portion of a complementary nucleic acid molecule, such as a mRNA or pre-mRNA corresponding to the coding strand of the human STXBP1 gene set forth in NCBI Accession No. NC_000009.12. In another embodiment, the ASOs provided herein target or bind to a portion of a complementary nucleic acid molecule, such as a mRNA or pre-mRNA corresponding to human STXBP1 gene splice variants NM_003165 or NM_001032221.

In one embodiment, the ASOs provided herein comprise a base sequence set forth in any one of SEQ ID NOs: 1-626. In one embodiment, the base sequence consists of a sequence set forth in any one of SEQ ID NOs: 1-626. In one embodiment, the ASO provided herein comprises a base sequence with at least 80%, 85%, 90% or 95% sequence identity to any one of SEQ ID NOs: 1-626 and when contacted with a cell increases expression of STXBP1 protein. In one embodiment, the cell is a neuronal cell, optionally in vivo or in vitro, and the ASO provided herein increases a level of functional STXBP1 protein in the neuronal cell relative to a control cell not contacted with the ASO.

In another embodiment, the ASOs provided herein contain at least 10, 11, 12, 13, 14, 15 or 16 consecutive bases of one of the following sequences:

(SEQ ID NO: 1)
CAGAGGCCAGCTGACTGC,
(SEQ ID NO: 2)
TGTACTCACAGTCAGTG,
(SEQ ID NO: 3)
AGGAGGCAGCTTCCCTG,
(SEQ ID NO: 4)
ACTGACGCGCGGACTG,
(SEQ ID NO: 5)
CTGCGCGAGTCTCCCG,
(SEQ ID NO: 6)
TGCCAGCCAGGGCGTGCAGG,
(SEQ ID NO: 7)
CGAGAATGCAGCGGCAACAG,
(SEQ ID NO: 8)
AACAGTCCAGAAATTTCTCC,
(SEQ ID NO: 9)
CCAAGCAATGTGCACGTCAC,
(SEQ ID NO: 10)
CAAAGAGATGGAGGCTTCCA,
(SEQ ID NO: 17)
CAGAGGCCAGCTGACTG,
(SEQ ID NO: 75)
AGGAGGCAGCTTCCCT,
(SEQ ID NO: 168)
CACGAGAATGCAGCGGCA,
(SEQ ID NO: 188)
CCAGAAATTTCTCCTG,
(SEQ ID NO: 197)
CAAGCAATGTGCACGTCACC,
and
(SEQ ID NO: 225)
TTCCAAAGAGATGGAGGCTT

Without being bound by any theory, it is believed that SEQ ID NOs: 1-3, 17 and 75 target a splice site and/or an exon splicing enhancer (ESE) of a mutant STXBP1 pre-mRNA, and/or induce exon skipping or inclusion in a mutant STXBP1 pre-mRNA. Without being bound by any theory, it is believed that SEQ ID NOs: 4 and 5 target a 5′-UTR of an STXBP1 mRNA. Without being bound by any theory, it is believed that SEQ ID NOs: 6-10, 168, 188, 197 and 225 target a 3′-UTR of an STXBP1 mRNA.

In all embodiments herein, the ASOs provided herein are complementary or reverse complementary to their intended mRNA or pre-mRNA target. The terms complementarity and reverse complementarity are used herein to refer to a stretch of nucleic acids that can hybridize to another stretch of nucleic acids under physiological conditions in a cell. Such hybridization generally follows well-known A-T(U)/G-C base pairing. Thus, reference to T herein also includes the corresponding ASO containing a U instead of the T. In one embodiment, the ASOs provided herein are fully T containing ASOs. In another embodiment, the ASOs provided herein are fully U containing ASOs. In another embodiment, the ASOs provided herein contain a mixture of T and U bases. In one embodiment, the ASOs provided herein are fully complementary or reverse complementary to their target. In another embodiment, the ASOs provided herein are less than fully complementary or reverse complementary to their target. In such embodiments, such “mismatches” are within the scope of this disclosure to the extent that the resulting ASO hybridizes to its target under physiological conditions in a cell. In certain embodiments, the degree of complementarity or reverse complementarity of the ASO provided herein to its target is at least 85%, 90%, 95%, 96%, 97%, 98%, 99% or is 100%. The ASOs specifically disclosed herein, represented by SEQ ID NOs, are 100% reverse complementary to their target. Thus, unless otherwise indicated, recitation of a specific SEQ ID NO herein, which is 100% complementary or reverse complementary to its target, also encompasses ASOs containing mismatches that are at least 85%, 90%, 95%, 96%, 97%, 98%, 99% complementary or reverse complementary to the same target. In one embodiment, the ASOs provided herein have 1 or 2 mismatches to their target, such as a STXBP1 mRNA or pre-mRNA sequence.

In one embodiment, the ASO provided herein has a length of less than 50 nucleotides, or from 8-40, 10-33 or 15-25 nucleotides. In another embodiment, the ASO provided herein has a length of 12-30 nucleotides. In another embodiment, the ASO provided herein has a length of 16-25 nucleotides. In another embodiment, the ASO provided herein has a length of 16-20 nucleotides. In another embodiment, the ASO provided herein has a length of 16, 17, 18, 19 or 20 nucleotides.

In one embodiment, the ASO provided herein has one or more modified bases, one or more modified sugars, and/or one or more modified internucleoside linkages. In another embodiment, the ASO provided herein has one or more modified bases. In another embodiment, the ASO provided herein has one or more modified sugars. In another embodiment, the ASO provided herein has one or more modified internucleoside linkages.

In another embodiment, the ASO provided herein has one or more modified bases selected from hypoxanthine, pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-methylcytosine, 5-propynylcytosine, 5-aminomethyluracil, 5-hydroxymethyluracil, 5-aminomethylcytosine, 5-hydroxymethylcytosine, 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, pseudoisocytosine, N⁴-ethylcytosine, N²-cyclopentylguanine (cPent-G), N²-cyclopentyl-2-aminopurine (cPent-AP), or N²-propyl-2-aminopurine (Pr-AP).

In another embodiment, the ASO provided herein has one or more modified sugars selected from 2′-substituted RNA sugars including threose nucleic acid sugars, 2′-F sugars, 2′-O-substituted RNA sugars including 2′-OMe and 2′-MOE (2′-O-(2-methoxyethyl)) sugars, and a bicyclic (BNA) or tricyclic (TNA) nucleic acid sugars. In another embodiment, the ASO provided herein has one or more BNAs and/or TNAs selected from a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNANC(N—H) monomer, a 2′,4′-BNANC(N-Me) monomer, a 2′,4′-BNANC(N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer (such as triazolyl or tetrazolyl-linked), an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an oxetane nucleotide monomer, a locked PMO monomer derived from 2′-amino-LNA, a guanidine-bridged nucleic acid (GuNA) monomer, a spirocyclopropylene-bridged nucleic acid (scpBNA) monomer, and derivatives thereof. In one embodiment, the ASO provided herein has more than one distinct modified sugar. In another embodiment, the ASO provided herein has a combination of 2′-O-substituted RNA sugars and BNAs. In another embodiment, the ASO provided herein has a combination of 2′-MOE and LNA sugars. In another embodiment, the ASO provided herein has all 2′-MOE sugars. See, e.g., Seth et al., J. Org. Chem. 2010, 75, 1569-1581; Osawa et al., J. Org. Chem., 2015, 80 (21), pp 10474-10481; WO 2014/145356; WO 2014/126229; Yamamoto et al. Org. Biomol. Chem. 2015, 13, 3757; Nishida et al. Chem. Commun. 2010, 46, 5283; WO 2015/142910; Hanessian et al., J. Org. Chem., 2013, 78 (18), pp 9064-9075; Bolli et al., Chem Biol. 1996 March; 3(3):197-206; Murray et al., Nucl. Acids Res., 2012, Vol. 40, No. 13 6135-6143; WO 2011/097641 and WO 2016/017422.

In another embodiment, the ASO provided herein has a modified internucleoside linkage, such as a phosphorothioate or phosphoroamidate. In another embodiment, the ASO provided herein has a phosphorothioate internucleoside linkage. In another embodiment, the ASO provided herein has at least 50%, 60%, 70%, 80%, 90% or all phosphorothioate internucleoside linkages.

In another embodiment, the ASO provided herein is a phosphorodiamidate morpholino oligomer (PMO), or is incorporated into a conjugate, such as a peptide-conjugated PMO (PPMO) or an ASO-antibody conjugate. See, e.g., WO 2022/192749, WO 2020/028832, WO 2021/142307, WO 2022/020107, WO 2022/140535.

In another embodiment, the ASO provided herein has a combination of 2′-MOE and LNA sugars and all phosphorothioate internucleoside linkages. In another embodiment, the ASO provided herein has all 2′-MOE sugars and all phosphorothioate internucleoside linkages.

In another embodiment, the ASO provided herein contains at least 10, 11, 12, 13, 14, 15 or 16 consecutive bases of CTGCGCGAGTCTCCCG (SEQ ID NO: 1), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CTGCGCGAGTCTCCCG (SEQ ID NO: 1), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CTGCGCGAGTCTCCCG (SEQ ID NO: 1), and optionally has all 2′-MOE sugars and all phosphorothioate internucleoside linkages. In another embodiment, the ASO provided herein contains at least 10, 11, 12, 13, 14, 15 or 16 consecutive bases of CTGCGCGAGTCTCCCG (SEQ ID NO: 1), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CTGCGCGAGTCTCCCG (SEQ ID NO: 1), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CTGCGCGAGTCTCCCG (SEQ ID NO: 1), has all 2′-MOE sugars and all phosphorothioate internucleoside linkages.

In another embodiment, the ASO provided herein contains at least 10, 11, 12, 13, 14, 15 or 16 consecutive bases of TGTACTCACAGTCAGTG (SEQ ID NO: 2), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TGTACTCACAGTCAGTG (SEQ ID NO: 2), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TGTACTCACAGTCAGTG (SEQ ID NO: 2), and optionally has all 2′-MOE sugars and all phosphorothioate internucleoside linkages. In another embodiment, the ASO provided herein contains at least 10, 11, 12, 13, 14, 15 or 16 consecutive bases of TGTACTCACAGTCAGTG (SEQ ID NO: 2), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TGTACTCACAGTCAGTG (SEQ ID NO: 2), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TGTACTCACAGTCAGTG (SEQ ID NO: 2), has all 2′-MOE sugars and all phosphorothioate internucleoside linkages.

In another embodiment, the ASO provided herein contains at least 10, 11, 12, 13, 14, 15 or 16 consecutive bases of AGGAGGCAGCTTCCCTG (SEQ ID NO: 3), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AGGAGGCAGCTTCCCTG (SEQ ID NO: 3), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AGGAGGCAGCTTCCCTG (SEQ ID NO: 3), and optionally has all 2′-MOE sugars and all phosphorothioate internucleoside linkages. In another embodiment, the ASO provided herein contains at least 10, 11, 12, 13, 14, 15 or 16 consecutive bases of AGGAGGCAGCTTCCCTG (SEQ ID NO: 3), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AGGAGGCAGCTTCCCTG (SEQ ID NO: 3), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AGGAGGCAGCTTCCCTG (SEQ ID NO: 3), has all 2′-MOE sugars and all phosphorothioate internucleoside linkages.

In another embodiment, the ASO provided herein contains at least 10, 11, 12, 13, 14, 15 or 16 consecutive bases of ACTGACGCGCGGACTG (SEQ ID NO: 4), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of ACTGACGCGCGGACTG (SEQ ID NO: 4), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of ACTGACGCGCGGACTG (SEQ ID NO: 4), and optionally has all 2′-MOE sugars and all phosphorothioate internucleoside linkages. In another embodiment, the ASO provided herein contains at least 10, 11, 12, 13, 14, 15 or 16 consecutive bases of ACTGACGCGCGGACTG (SEQ ID NO: 4), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of ACTGACGCGCGGACTG (SEQ ID NO: 4), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of ACTGACGCGCGGACTG (SEQ ID NO: 4), has all 2′-MOE sugars and all phosphorothioate internucleoside linkages.

In another embodiment, the ASO provided herein contains at least 10, 11, 12, 13, 14, 15 or 16 consecutive bases of CTGCGCGAGTCTCCCG (SEQ ID NO: 5), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CTGCGCGAGTCTCCCG (SEQ ID NO: 5), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CTGCGCGAGTCTCCCG (SEQ ID NO: 5), and optionally has all 2′-MOE sugars and all phosphorothioate internucleoside linkages. In another embodiment, the ASO provided herein contains at least 10, 11, 12, 13, 14, 15 or 16 consecutive bases of CTGCGCGAGTCTCCCG (SEQ ID NO: 5), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CTGCGCGAGTCTCCCG (SEQ ID NO: 5), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CTGCGCGAGTCTCCCG (SEQ ID NO: 5), has all 2′-MOE sugars and all phosphorothioate internucleoside linkages.

In another embodiment, the ASO provided herein contains at least 10, 11, 12, 13, 14, 15 or 16 consecutive bases of TGCCAGCCAGGGCGTGCAGG (SEQ ID NO: 6), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TGCCAGCCAGGGCGTGCAGG (SEQ ID NO: 6), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TGCCAGCCAGGGCGTGCAGG (SEQ ID NO: 6), and optionally has all 2′-MOE sugars and all phosphorothioate internucleoside linkages. In another embodiment, the ASO provided herein contains at least 10, 11, 12, 13, 14, 15 or 16 consecutive bases of TGCCAGCCAGGGCGTGCAGG (SEQ ID NO: 6), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TGCCAGCCAGGGCGTGCAGG (SEQ ID NO: 6), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TGCCAGCCAGGGCGTGCAGG (SEQ ID NO: 6), has all 2′-MOE sugars and all phosphorothioate internucleoside linkages.

In another embodiment, the ASO provided herein contains at least 10, 11, 12, 13, 14, 15 or 16 consecutive bases of CGAGAATGCAGCGGCAACAG (SEQ ID NO: 7), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CGAGAATGCAGCGGCAACAG (SEQ ID NO: 7), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CGAGAATGCAGCGGCAACAG (SEQ ID NO: 7), and optionally has all 2′-MOE sugars and all phosphorothioate internucleoside linkages. In another embodiment, the ASO provided herein contains at least 10, 11, 12, 13, 14, 15 or 16 consecutive bases of CGAGAATGCAGCGGCAACAG (SEQ ID NO: 7), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CGAGAATGCAGCGGCAACAG (SEQ ID NO: 7), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CGAGAATGCAGCGGCAACAG (SEQ ID NO: 7), has all 2′-MOE sugars and all phosphorothioate internucleoside linkages.

In another embodiment, the ASO provided herein contains at least 10, 11, 12, 13, 14, 15 or 16 consecutive bases of AACAGTCCAGAAATTTCTCC (SEQ ID NO: 8), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AACAGTCCAGAAATTTCTCC (SEQ ID NO: 8), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AACAGTCCAGAAATTTCTCC (SEQ ID NO: 8), and optionally has all 2′-MOE sugars and all phosphorothioate internucleoside linkages. In another embodiment, the ASO provided herein contains at least 10, 11, 12, 13, 14, 15 or 16 consecutive bases of AACAGTCCAGAAATTTCTCC (SEQ ID NO: 8), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AACAGTCCAGAAATTTCTCC (SEQ ID NO: 8), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AACAGTCCAGAAATTTCTCC (SEQ ID NO: 8), has all 2′-MOE sugars and all phosphorothioate internucleoside linkages.

In another embodiment, the ASO provided herein contains at least 10, 11, 12, 13, 14, 15 or 16 consecutive bases of CCAAGCAATGTGCACGTCAC (SEQ ID NO: 9), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CCAAGCAATGTGCACGTCAC (SEQ ID NO: 9), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CCAAGCAATGTGCACGTCAC (SEQ ID NO: 9), and optionally has all 2′-MOE sugars and all phosphorothioate internucleoside linkages. In another embodiment, the ASO provided herein contains at least 10, 11, 12, 13, 14, 15 or 16 consecutive bases of CCAAGCAATGTGCACGTCAC (SEQ ID NO: 9), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CCAAGCAATGTGCACGTCAC (SEQ ID NO: 9), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CCAAGCAATGTGCACGTCAC (SEQ ID NO: 9), has all 2′-MOE sugars and all phosphorothioate internucleoside linkages.

In another embodiment, the ASO provided herein contains at least 10, 11, 12, 13, 14, 15 or 16 consecutive bases of CAAAGAGATGGAGGCTTCCA (SEQ ID NO: 10), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAAAGAGATGGAGGCTTCCA (SEQ ID NO: 10), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAAAGAGATGGAGGCTTCCA (SEQ ID NO: 10), and optionally has all 2′-MOE sugars and all phosphorothioate internucleoside linkages. In another embodiment, the ASO provided herein contains at least 10, 11, 12, 13, 14, 15 or 16 consecutive bases of CAAAGAGATGGAGGCTTCCA (SEQ ID NO: 10), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAAAGAGATGGAGGCTTCCA (SEQ ID NO: 10), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAAAGAGATGGAGGCTTCCA (SEQ ID NO: 10), has all 2′-MOE sugars and all phosphorothioate internucleoside linkages.

In another embodiment, the ASO provided herein contains at least 10, 11, 12, 13, 14, 15 or 16 consecutive bases of CAGAGGCCAGCTGACTG (SEQ ID NO: 17), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAGAGGCCAGCTGACTG (SEQ ID NO: 17), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAGAGGCCAGCTGACTG (SEQ ID NO: 17), and optionally has all 2′-MOE sugars and all phosphorothioate internucleoside linkages. In another embodiment, the ASO provided herein contains at least 10, 11, 12, 13, 14, 15 or 16 consecutive bases of CAGAGGCCAGCTGACTG (SEQ ID NO: 17), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAGAGGCCAGCTGACTG (SEQ ID NO: 17), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAGAGGCCAGCTGACTG (SEQ ID NO: 17), has all 2′-MOE sugars and all phosphorothioate internucleoside linkages.

In another embodiment, the ASO provided herein contains at least 10, 11, 12, 13, 14, 15 or 16 consecutive bases of AGGAGGCAGCTTCCCT (SEQ ID NO: 75), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AGGAGGCAGCTTCCCT (SEQ ID NO: 75), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AGGAGGCAGCTTCCCT (SEQ ID NO: 75), and optionally has all 2′-MOE sugars and all phosphorothioate internucleoside linkages. In another embodiment, the ASO provided herein contains at least 10, 11, 12, 13, 14, 15 or 16 consecutive bases of AGGAGGCAGCTTCCCT (SEQ ID NO: 75), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of AGGAGGCAGCTTCCCT (SEQ ID NO: 75), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of AGGAGGCAGCTTCCCT (SEQ ID NO: 75), has all 2′-MOE sugars and all phosphorothioate internucleoside linkages.

In another embodiment, the ASO provided herein contains at least 10, 11, 12, 13, 14, 15 or 16 consecutive bases of CACGAGAATGCAGCGGCA (SEQ ID NO: 168), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CACGAGAATGCAGCGGCA (SEQ ID NO: 168), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CACGAGAATGCAGCGGCA (SEQ ID NO: 168), and optionally has all 2′-MOE sugars and all phosphorothioate internucleoside linkages. In another embodiment, the ASO provided herein contains at least 10, 11, 12, 13, 14, 15 or 16 consecutive bases of CACGAGAATGCAGCGGCA (SEQ ID NO: 168), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CACGAGAATGCAGCGGCA (SEQ ID NO: 168), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CACGAGAATGCAGCGGCA (SEQ ID NO: 168), has all 2′-MOE sugars and all phosphorothioate internucleoside linkages.

In another embodiment, the ASO provided herein contains at least 10, 11, 12, 13, 14, 15 or 16 consecutive bases of CCAGAAATTTCTCCTG (SEQ ID NO: 188), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CCAGAAATTTCTCCTG (SEQ ID NO: 188), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CCAGAAATTTCTCCTG (SEQ ID NO: 188), and optionally has all 2′-MOE sugars and all phosphorothioate internucleoside linkages. In another embodiment, the ASO provided herein contains at least 10, 11, 12, 13, 14, 15 or 16 consecutive bases of CCAGAAATTTCTCCTG (SEQ ID NO: 188), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CCAGAAATTTCTCCTG (SEQ ID NO: 188), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CCAGAAATTTCTCCTG (SEQ ID NO: 188), has all 2′-MOE sugars and all phosphorothioate internucleoside linkages.

In another embodiment, the ASO provided herein contains at least 10, 11, 12, 13, 14, 15 or 16 consecutive bases of CAAGCAATGTGCACGTCACC (SEQ ID NO: 197), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAAGCAATGTGCACGTCACC (SEQ ID NO: 197), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAAGCAATGTGCACGTCACC (SEQ ID NO: 197), and optionally has all 2′-MOE sugars and all phosphorothioate internucleoside linkages. In another embodiment, the ASO provided herein contains at least 10, 11, 12, 13, 14, 15 or 16 consecutive bases of CAAGCAATGTGCACGTCACC (SEQ ID NO: 197), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of CAAGCAATGTGCACGTCACC (SEQ ID NO: 197), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of CAAGCAATGTGCACGTCACC (SEQ ID NO: 197), has all 2′-MOE sugars and all phosphorothioate internucleoside linkages.

In another embodiment, the ASO provided herein contains at least 10, 11, 12, 13, 14, 15 or 16 consecutive bases of TTCCAAAGAGATGGAGGCTT (SEQ ID NO: 225), and optionally has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TTCCAAAGAGATGGAGGCTT (SEQ ID NO: 225), and optionally has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TTCCAAAGAGATGGAGGCTT (SEQ ID NO: 225), and optionally has all 2′-MOE sugars and all phosphorothioate internucleoside linkages. In another embodiment, the ASO provided herein contains at least 10, 11, 12, 13, 14, 15 or 16 consecutive bases of TTCCAAAGAGATGGAGGCTT (SEQ ID NO: 225), has all 2′-MOE sugars and all phosphorothioate internucleoside linkages. In another embodiment, the ASO provided herein is 16-25 nucleotides in length, contains at least 10, 11, 12, 13, 14, 15 or 16 consecutive bases of TTCCAAAGAGATGGAGGCTT (SEQ ID NO: 225), has all 2′-MOE sugars and all phosphorothioate internucleoside linkages. In another embodiment, the ASO provided herein is 16-20 nucleotides in length, contains at least 10, 11, 12, 13, 14, 15 or 16 consecutive bases of TTCCAAAGAGATGGAGGCTT (SEQ ID NO: 225), has all 2′-MOE sugars and all phosphorothioate internucleoside linkages.

In one embodiment, provided herein is an ASO comprising a base sequence with at least 80%, 85%, 90%, 95% or 100% sequence identity to a sequence set forth in any one of Tables 1-4. In another embodiment, provided herein is an ASO comprising at least 8, 9, 10, 11, 12, 13, 14, 15, 16, 17, 18, 19 or 20 consecutive bases of a sequence set forth in any one of Tables 1-4. In another embodiment, provided herein is an ASO comprising between 16-25 nucleotides and containing at least 8, 9, 10, 11, 12, 13, 14, 15, 17, 17, 18 19 or 20 consecutive bases of any one of SEQ ID Nos: 1-626.

In one embodiment, provided herein is an ASO comprising a base sequence set forth in Table 1 or Table 2. In another embodiment, provided herein is an ASO comprising a base sequence selected from SEQ ID NOs: 2, 17, 75, 168, 188, 197 and 225.

In one embodiment, the ASO provided herein comprises one or more modified bases, one or more modified sugars, and/or one or more modified internucleoside linkages. In another embodiment, the ASO provided herein comprises one or more 2′-MOE sugars and one or more phosphorothioate internucleoside linkages. In one embodiment, at least 50%, 75%, 90% or all of the sugars in the ASO provided herein are 2′-MOE sugars and, independently, at least 50%, 75%, 90% or all of the internucleoside linkages in the ASO provided herein are phosphorothioate internucleoside linkages.

In one embodiment, the ASO provided herein comprises a base sequence selected from those in Table 1, optionally wherein the ASO has one or more modified bases, one or more modified sugars, and/or one or more modified internucleoside linkages. In another embodiment, the ASO provided herein has all 2′-MOE sugars and all phosphorothioate internucleoside linkages, and has a base sequence selected from those in Table 1:

TABLE 1
ASO sequence

| SEQ ID NO: | Base Sequence |
|---|---|
| 11 | AGAGGCCAGCTGACTGCTT |
| 12 | ACAGAGGCCAGCTGACTGC |
| 13 | AGAACAGAGGCCAGCTG |
| 14 | GAACAGAGGCCAGCTGA |
| 15 | AACAGAGGCCAGCTGAC |
| 16 | ACAGAGGCCAGCTGACT |
| 17 | CAGAGGCCAGCTGACTG |
| 18 | AGAGGCCAGCTGACTGC |
| 19 | GAGGCCAGCTGACTGCT |
| 20 | CAGAGGCCAGCTGACTGCT |
| 21 | AGGCCAGCTGACTGCTT |
| 22 | AGAACAGAGGCCAGCTGA |
| 23 | GAACAGAGGCCAGCTGAC |
| 24 | AACAGAGGCCAGCTGACT |
| 25 | ACAGAGGCCAGCTGACTG |
| 26 | AGAGGCCAGCTGACTGCT |
| 27 | GAGGCCAGCTGACTGCTT |
| 28 | AGGCCAGCTGACTGCTTG |
| 29 | GGCCAGCTGACTGCTTGA |
| 30 | GGCCAGCTGACTGCTTG |
| 31 | GAACAGAGGCCAGCTGACT |
| 32 | GAGGCCAGCTGACTGCTTG |
| 33 | AACAGAGGCCAGCTGACTG |
| 34 | GCCAGCTGACTGCTTGA |
| 35 | ACTCACAGTCAGTGAA |
| 36 | TACTCACAGTCAGTGA |
| 37 | GTACTCACAGTCAGTG |
| 38 | TGTACTCACAGTCAGT |
| 39 | TTGTACTCACAGTCAG |
| 40 | GTTGTACTCACAGTCA |
| 41 | GGTTGTACTCACAGTC |
| 42 | TGGTTGTACTCACAGT |
| 43 | TGTACTCACAGTCAGTGAAG |
| 44 | TTGTACTCACAGTCAGTGAA |
| 45 | GTTGTACTCACAGTCAGT |
| 46 | TTGTACTCACAGTCAGTG |
| 47 | TGTACTCACAGTCAGTGA |
| 48 | GTACTCACAGTCAGTGAA |
| 49 | TACTCACAGTCAGTGAAG |
| 50 | ACTCACAGTCAGTGAAGA |
| 51 | GGTTGTACTCACAGTCAGTG |
| 52 | GTTGTACTCACAGTCAGTGA |
| 53 | CTCACAGTCAGTGAAG |
| 54 | GGTTGTACTCACAGTCAG |
| 55 | TGGTTGTACTCACAGTCA |
| 56 | TCACAGTCAGTGAAGA |
| 57 | AGGAGGCAGCTTCCCTGC |
| 58 | GAGGCAGCTTCCCTGCTC |
| 59 | GGAGGCAGCTTCCCTGCT |
| 60 | GAGGAGGCAGCTTCCCTG |
| 61 | GGAGGAGGCAGCTTCCCT |
| 62 | TGGAGGAGGCAGCTTCCC |
| 63 | GGCAGCTTCCCTGCTCT |
| 64 | AGGCAGCTTCCCTGCTC |
| 65 | GAGGCAGCTTCCCTGCT |
| 66 | GAGGAGGCAGCTTCCCT |
| 67 | GGAGGAGGCAGCTTCCC |
| 68 | TGGAGGAGGCAGCTTCC |
| 69 | TTGGAGGAGGCAGCTTC |
| 70 | GGAGGCAGCTTCCCTGC |
| 71 | GGCAGCTTCCCTGCTC |
| 72 | AGGCAGCTTCCCTGCT |
| 73 | GAGGCAGCTTCCCTGC |
| 74 | GGAGGCAGCTTCCCTG |
| 75 | AGGAGGCAGCTTCCCT |
| 76 | GAGGAGGCAGCTTCCC |
| 77 | GGAGGAGGCAGCTTCC |
| 78 | TGGAGGAGGCAGCTTC |
| 79 | TTGGAGGAGGCAGCTT |
| 80 | GCAGCTTCCCTGCTCT |
| 81 | ACGCGCGGACTGGGC |
| 82 | GACGCGCGGACTGGG |
| 83 | TGACGCGCGGACTGG |
| 84 | CTGACGCGCGGACTG |
| 85 | ACTGACGCGCGGACT |
| 86 | CCGACTGACGCGCGG |
| 87 | CGACTGACGCGCGGA |
| 88 | ACCGACTGACGCGCGG |
| 89 | ACGCGCGGACTGGGCG |
| 90 | CCGACTGACGCGCGGA |
| 91 | GACTGACGCGCGGAC |
| 92 | CGACTGACGCGCGGAC |
| 93 | TGACGCGCGGACTGGGC |
| 94 | CTGACGCGCGGACTGG |
| 95 | TGACGCGCGGACTGGG |
| 96 | GACGCGCGGACTGGGC |
| 97 | ACCGACTGACGCGCGGA |
| 98 | CCGACTGACGCGCGGAC |
| 99 | CGACTGACGCGCGGACT |
| 100 | GACTGACGCGCGGACTG |
| 101 | GACGCGCGGACTGGGCG |
| 102 | ACTGACGCGCGGACTGG |
| 103 | CTGACGCGCGGACTGGG |
| 104 | GACTGACGCGCGGACT |
| 105 | GCGAGTCTCCCGGCTC |
| 106 | GCGCTGCGCGAGTCTC |
| 107 | GGCGCTGCGCGAGTCT |
| 108 | CGCTGCGCGAGTCTCCCGGC |
| 109 | CGCTGCGCGAGTCTCC |
| 110 | GCTGCGCGAGTCTCCCGGCT |
| 111 | CGCGAGTCTCCCGGCT |
| 112 | GCTGCGCGAGTCTCCC |
| 113 | TGCGCGAGTCTCCCGG |
| 114 | GCGCGAGTCTCCCGGC |
| 115 | GCGCTGCGCGAGTCTCCCGG |
| 116 | GCGCTGCGCGAGTCTCCC |
| 117 | CGCTGCGCGAGTCTCCCG |
| 118 | GCTGCGCGAGTCTCCCGG |
| 119 | CTGCGCGAGTCTCCCGGC |
| 120 | TGCGCGAGTCTCCCGGCT |
| 121 | GGCGCTGCGCGAGTCTCC |
| 122 | GCGCGAGTCTCCCGGCTC |
| 123 | AGCCAGGGCGTGCAGGGC |
| 124 | GCCAGGGCGTGCAGGGCT |
| 125 | CAGTGCCAGCCAGGGCGTGC |
| 126 | AGTGCCAGCCAGGGCGTGCA |
| 127 | GCCAGCCAGGGCGTGCAGGG |
| 128 | CCAGCCAGGGCGTGCAGGGC |
| 129 | CAGCCAGGGCGTGCAGGGCT |
| 130 | CAGCCAGGGCGTGCAGGG |
| 131 | GTGCCAGCCAGGGCGTGCAG |
| 132 | CCAGCCAGGGCGTGCAGG |
| 133 | GCCAGGGCGTGCAGGG |
| 134 | TGCCAGCCAGGGCGTGCA |
| 135 | AGTGCCAGCCAGGGCG |
| 136 | GCCAGCCAGGGCGTGCAG |
| 137 | TGCCAGCCAGGGCGTG |
| 138 | GCCAGCCAGGGCGTGC |
| 139 | CCAGCCAGGGCGTGCA |
| 140 | GTGCCAGCCAGGGCGT |
| 141 | AGCCAGGGCGTGCAGG |
| 142 | CCAGGGCGTGCAGGGC |
| 143 | CAGTGCCAGCCAGGGCGT |
| 144 | AGTGCCAGCCAGGGCGTG |
| 145 | GTGCCAGCCAGGGCGTGC |
| 146 | CAGCCAGGGCGTGCAG |
| 147 | GAATGCAGCGGCAACAGT |
| 148 | GAATGCAGCGGCAACAGTGC |
| 149 | AGAATGCAGCGGCAACAGTG |
| 150 | GAGAATGCAGCGGCAACAGT |
| 151 | ACGAGAATGCAGCGGCAACA |
| 152 | CACGAGAATGCAGCGGCAAC |
| 153 | ACACGAGAATGCAGCGGCAA |
| 154 | ATGCAGCGGCAACAGTGC |
| 155 | AATGCAGCGGCAACAGTG |
| 156 | AGAATGCAGCGGCAACAG |
| 157 | GAGAATGCAGCGGCAACA |
| 158 | CGAGAATGCAGCGGCAAC |
| 159 | CACGAGAATGCAGCGG |
| 160 | ACGAGAATGCAGCGGC |
| 161 | GAGAATGCAGCGGCAA |
| 162 | AGAATGCAGCGGCAAC |
| 163 | GAATGCAGCGGCAACA |
| 164 | CGAGAATGCAGCGGCA |
| 165 | ATGCAGCGGCAACAGT |
| 166 | TGCAGCGGCAACAGTG |
| 167 | ACACGAGAATGCAGCGGC |
| 168 | CACGAGAATGCAGCGGCA |
| 169 | ACGAGAATGCAGCGGCAA |
| 170 | AATGCAGCGGCAACAG |
| 171 | AGTCCAGAAATTTCTCCT |
| 172 | AGTCCAGAAATTTCTCCTGC |
| 173 | CAGTCCAGAAATTTCTCCTG |
| 174 | TAACAGTCCAGAAATTTCTC |
| 175 | CTAACAGTCCAGAAATTTCT |
| 176 | CCTAACAGTCCAGAAATTTC |
| 177 | TCCAGAAATTTCTCCTGC |
| 178 | GTCCAGAAATTTCTCCTG |
| 179 | CAGTCCAGAAATTTCTCC |
| 180 | ACAGTCCAGAAATTTCTCCT |
| 18 | AACAGTCCAGAAATTTCT |
| 182 | CTAACAGTCCAGAAAT |
| 183 | TAACAGTCCAGAAATT |
| 184 | ACAGTCCAGAAATTTCTC |
| 185 | AACAGTCCAGAAATTT |
| 186 | ACAGTCCAGAAATTTC |
| 187 | CAGTCCAGAAATTTCT |
| 188 | CCAGAAATTTCTCCTG |
| 189 | GTCCAGAAATTTCTCC |
| 190 | TCCAGAAATTTCTCCT |
| 191 | TAACAGTCCAGAAATTTC |
| 192 | CTAACAGTCCAGAAATTT |
| 193 | AGTCCAGAAATTTCTC |
| 194 | CCTAACAGTCCAGAAATT |
| 195 | AGCAATGTGCACGTCACCCA |
| 196 | AAGCAATGTGCACGTCACCC |
| 197 | CAAGCAATGTGCACGTCACC |
| 198 | ACCCAAGCAATGTGCACGTC |
| 199 | TACCCAAGCAATGTGCACGT |
| 200 | CAATGTGCACGTCACCCA |
| 201 | GCAATGTGCACGTCACCC |
| 202 | AGCAATGTGCACGTCACC |
| 203 | AAGCAATGTGCACGTCAC |
| 204 | CCCAAGCAATGTGCACGTCA |
| 205 | CCAAGCAATGTGCACGTC |
| 206 | CAAGCAATGTGCACGTCA |
| 207 | CCCAAGCAATGTGCAC |
| 208 | CCAAGCAATGTGCACG |
| 209 | CAAGCAATGTGCACGT |
| 210 | AAGCAATGTGCACGTC |
| 211 | AGCAATGTGCACGTCA |
| 212 | ACCCAAGCAATGTGCA |
| 213 | CAATGTGCACGTCACC |
| 214 | AATGTGCACGTCACCC |
| 215 | TACCCAAGCAATGTGCAC |
| 216 | ACCCAAGCAATGTGCACG |
| 217 | CCCAAGCAATGTGCACGT |
| 218 | GCAATGTGCACGTCAC |
| 219 | AGAGATGGAGGCTTCCAG |
| 220 | AGAGATGGAGGCTTCCAGCG |
| 221 | AAGAGATGGAGGCTTCCAGC |
| 222 | AAAGAGATGGAGGCTTCCAG |
| 223 | CCAAAGAGATGGAGGCTTCC |
| 224 | TCCAAAGAGATGGAGGCTTC |
| 225 | TTCCAAAGAGATGGAGGCTT |
| 226 | AGATGGAGGCTTCCAGCG |
| 227 | GAGATGGAGGCTTCCAGC |
| 228 | AAGAGATGGAGGCTTCCA |
| 229 | GATGGAGGCTTCCAGC |
| 230 | CAAAGAGATGGAGGCTTC |
| 231 | CCAAAGAGATGGAGGCTT |
| 232 | TCCAAAGAGATGGAGGCT |
| 233 | TTCCAAAGAGATGGAGGC |
| 234 | AGATGGAGGCTTCCAG |
| 235 | GAGATGGAGGCTTCCA |
| 236 | AGAGATGGAGGCTTCC |
| 237 | AAGAGATGGAGGCTTC |
| 238 | AAAGAGATGGAGGCTT |
| 239 | CAAAGAGATGGAGGCT |
| 240 | CCAAAGAGATGGAGGC |
| 241 | AAAGAGATGGAGGCTTCC |
| 242 | TCCAAAGAGATGGAGG |

In one embodiment, the ASO provided herein comprises a base sequence selected from those in Table 2, optionally wherein the ASO has one or more modified bases, one or more modified sugars, and/or one or more modified internucleoside linkages. In another embodiment, the ASO provided herein has all 2′-MOE sugars and all phosphorothioate internucleoside linkages, and has a base sequence selected from those in Table 2:

TABLE 2
ASO Sequence

| SEQ ID NO: | Base Sequence |
|---|---|
| 3 | AGGAGGCAGCTTCCCTG |
| 243 | CCACTAGTCCTGTTTGGA |
| 244 | TTTGGAGGAGGCAGCTTCCC |
| 245 | TTCCTCTAGGTCCCACAGC |
| 246 | TTGTGCTTCAGTACTTTC |
| 247 | TTGTGCTTCAGTACTTTCAA |
| 248 | CAGCAGCACAAATGGTGT |
| 249 | GAGCGAGGCTGCCCCG |
| 250 | GCCAGAGCGAGGCTGC |
| 251 | GGCGCGGCGCGAGCCA |
| 252 | GCGGACTGGGCGCGGG |
| 253 | ACGCGCGGACTGGGCG |
| 4 | ACTGACGCGCGGACTG |
| 254 | ACCGACTGACGCGCGG |
| 255 | CGCAGCCGCGCTAGGG |
| 256 | GCCCCGCAGCCGCGCT |
| 257 | CCAGCCGCAGCTCTCC |
| 258 | GCCTGCGGACCCCGCC |
| 259 | CGACGCCTGCGGACCC |
| 260 | GCGAGTCTCCCGGCTC |
| 5 | CTGCGCGAGTCTCCCG |
| 6 | TGCCAGCCAGGGCGTGCAGG |
| 7 | CGAGAATGCAGCGGCAACAG |
| 261 | TTAAAAAACACAATGCATTA |
| 8 | AACAGTCCAGAAATTTCTCC |
| 9 | CCAAGCAATGTGCACGTCAC |
| 262 | GATCACAGCACTGTGGACAC |
| 263 | GACTGGAGGACTTCCCTCGG |
| 264 | TGTCAGGGCACTGTGACTTA |
| 265 | GCGGGAGTTTGCTTCTCACC |
| 10 | CAAAGAGATGGAGGCTTCCA |
| 266 | CGCGCTGGGCCAGCCG |
| 267 | GATCTCCTCGTCCCGCGA |
| 1 | CAGAGGCCAGCTGACTGC |
| 268 | GCAGTGGCTCACTCCT |
| 269 | TTTTGAGACAGTCTTG |
| 270 | CGGGCACGGTGGCTCA |
| 271 | CCCCTTCTTCTTGACCTTCTT |
| 272 | CAGGAGGACAGCATCCT |
| 273 | AAGGTTTACCTTCTCGG |
| 274 | GCCTCCAGGCTGGGGAGCGGCT |
| 275 | GATACACAGCCTCCAGGCTGGG |
| 276 | TTTACCTTCTCGGATGGAGTGA |
| 2 | TGTACTCACAGTCAGTG |
| 277 | TCCTGGATTAGCTGAGCCAGCA |
| 278 | CGAGCTTGTCCTGGATTAGCTG |
| 279 | CTAAGGGATTTTCACCTACCT |

In one embodiment, the ASO provided herein comprises a base sequence selected from those in Table 3, optionally wherein the ASO has one or more modified bases, one or more modified sugars, and/or one or more modified internucleoside linkages. In another embodiment, the ASO provided herein has all 2′-MOE sugars and all phosphorothioate internucleoside linkages, and has a base sequence selected from those in Table 3: Table 3: ASO sequence

TABLE 3
ASO sequence

| SEQ ID NO: | Base Sequence |
|---|---|
| 280 | AGCCGCAGCTCTCC |
| 281 | CTTCTTGACCTTCTTTAT |
| 271 | CCCCTTCTTCTTGACCTTCTT |
| 282 | TACTCACCTTCCATTCCCCCTT |
| 283 | AACTGTACTTTCTACTCACCTT |
| 272 | CAGGAGGACAGCATCCT |
| 284 | CAGCAGGAGGACAGCAT |
| 285 | CTCACTCGTTATGCCCTCGG |
| 286 | GCACCTGCAAGGAAAGAGAACA |
| 273 | AAGGTTTACCTTCTCGG |
| 274 | GCCTCCAGGCTGGGGAGCGGCT |
| 275 | GATACACAGCCTCCAGGCTGGG |
| 276 | TTTACCTTCTCGGATGGAGTGA |
| 287 | TAGCAGTCGGCGGGTCC |
| 2 | TGTACTCACAGTCAGTG |
| 288 | CGGTATTTAGCAGTCGGCGG |
| 289 | TACTCACAGTCAGTGAAGAAG |
| 290 | CAAGCCAGACTTACCTC |
| 291 | ACTTACCTCCCCCATTGTTG |
| 277 | TCCTGGATTAGCTGAGCCAGCA |
| 278 | CGAGCTTGTCCTGGATTAGCTG |
| 292 | TTCTCTCCAGTATTCAT |
| 293 | CCGGGTGACTTCCCTGGAAGA |
| 294 | TTTACCTTCTCTCCAGTATTC |
| 295 | CTCTCCAGTATTCATTCTCTTG |
| 296 | TTTGGTGTCAAGTTTGTCCT |
| 297 | GGTAGTGTTTGGTGTCAAGTT |
| 279 | CTAAGGGATTTTCACCTACCT |
| 298 | ACCTACCTGACGGCGGTGGTGC |
| 299 | AGAGAAAGCAGGGACTAA |
| 300 | AGGCAGCTTCCCTGCTCTTA |
| 301 | AAGACAGGGTCTGTGCC |
| 302 | GCCTCCTCAGGTGGGC |
| 303 | AGGAGTCATGCAATTTT |
| 304 | GCAACACCATGGACCTT |
| 305 | GGAAGATAAAAGGAGGC |
| 306 | AGCACTGGCATGGGTTT |
| 307 | TTACTGGGCATTTGAGC |
| 3 | AGGAGGCAGCTTCCCTG |
| 308 | GTCCTGTTTGGAGGAGG |
| 309 | TAGTCCTGTTTGGAGGA |
| 310 | ACCTTGCTATGGATGCTA |
| 311 | GTTTCACAGCAACACCAT |
| 312 | AGATAAAAGGAGGCAGCT |
| 313 | AAAGCAGGGACTAATTTA |
| 243 | CCACTAGTCCTGTTTGGA |
| 314 | AACACCATGGACCTTGCTA |
| 315 | TTTGAGCACTGGCATGGGT |
| 316 | CTGGGCATTTGAGCACTGGC |
| 317 | CTAATTTACTGGGCATTTGA |
| 244 | TTTGGAGGAGGCAGCTTCCC |
| 318 | GCAGGGACTAATTTACTGGGC |
| 319 | TGCTAACCCACTAGTCCTGTT |
| 320 | TTGGCTCCATCTCTCTG |
| 321 | AAGGAAAACACACCTCA |
| 322 | CTAGGTCCCACAGCCCT |
| 323 | TCTAGGTCCCACAGCCC |
| 324 | TTCTCAGTTGTCTCAGT |
| 325 | TTCTTAGGAAATTCAGGA |
| 326 | TGGAGGAACAGTAGTTCT |
| 327 | GGCTCCATCTCTCTGGTG |
| 328 | GCCTTCCTCTAGGTCCCA |
| 329 | TGCTTTGTTTCCTCTTTC |
| 330 | GGATCCACAAATGTCTAGA |
| 331 | AACAGTAGTTCTTAGGAAA |
| 245 | TTCCTCTAGGTCCCACAGC |
| 332 | TCTGTGCCTTCCTCTAGGT |
| 333 | ATGACTTCTCTGCTTTGTT |
| 334 | ACCTCATTGGCTCCATCTCT |
| 335 | GTCTTTTAAATGACTTCTCT |
| 336 | CTCTCTGGTGACAGTGTGGAG |
| 337 | AGGAAAACACACCTCATTGGC |
| 338 | TTTCCCCAAGACAGGGTCTGT |
| 339 | TCCTCTTTCTCAGTTGTCTCA |
| 246 | TTGTGCTTCAGTACTTTC |
| 340 | TATGTTTGTGCTTCAGTA |
| 341 | TGATATATGTTTGTGCTT |
| 342 | TTGTGCTTCAGTACTTTCAA |
| 343 | TATGTTTGTGCTTCAGTACT |
| 344 | TGATATATGTTTGTGCTTCA |
| 345 | TTGTGCTTCAGTACTTTCAAAC |
| 346 | TATGTTTGTGCTTCAGTACTTT |
| 347 | TGATATATGTTTGTGCTTCAGT |
| 248 | CAGCAGCACAAATGGTGT |
| 348 | AACAGCAGCAGCACAAAT |
| 349 | ACGACAACAGCAGCAGCA |
| 350 | CAGCAGCACAAATGGTGTGC |
| 351 | AACAGCAGCAGCACAAATGG |
| 352 | ACGACAACAGCAGCAGCACA |
| 353 | CAGCAGCACAAATGGTGTGCTC |
| 354 | AACAGCAGCAGCACAAATGGTG |
| 355 | ACGACAACAGCAGCAGCACAAA |
| 356 | GGGCGGGGCAAGCCGG |
| 357 | GCGGGGGCGGGGCAAG |
| 358 | GCGCGCGGGGGCGGGG |
| 359 | CGGCGCGCGCGGGGGC |
| 360 | CCGCCGGCGCGCGCGG |
| 361 | CCCGCCGCCGGCGCGC |
| 362 | CTGCCCCGCCGCCGGC |
| 363 | GAGGCTGCCCCGCCGC |
| 249 | GAGCGAGGCTGCCCCG |
| 364 | GCCAGAGCGAGGCTGC |
| 365 | GCGAGCCAGAGCGAGG |
| 366 | CGGCGCGAGCCAGAGC |
| 367 | GGCGCGGCGCGAGCCA |
| 368 | CGGGGGCGCGGCGCGA |
| 369 | GGCGCGGGGGCGCGGC |
| 370 | ACTGGGCGCGGGGGCG |
| 252 | GCGGACTGGGCGCGGG |
| 253 | ACGCGCGGACTGGGCG |
| 4 | ACTGACGCGCGGACTG |
| 254 | ACCGACTGACGCGCGG |
| 371 | AGGGACCGACTGACGC |
| 372 | CGCTAGGGACCGACTG |
| 373 | GCCGCGCTAGGGACCG |
| 255 | CGCAGCCGCGCTAGGG |
| 256 | GCCCCGCAGCCGCGCT |
| 374 | CTCCGCCCCGCAGCCG |
| 375 | AGCTCTCCGCCCCGCA |
| 376 | CCGCAGCTCTCCGCCC |
| 257 | CCAGCCGCAGCTCTCC |
| 377 | TGGGCCAGCCGCAGCT |
| 378 | GCGCTGGGCCAGCCGC |
| 379 | GGGCGCGCTGGGCCAG |
| 380 | AGGTGGGCGCGCTGGG |
| 381 | CCTCAGGTGGGCGCGC |
| 382 | CGCCGCCTCCTCAGGT |
| 383 | ACCCCGCCGCCTCCTC |
| 384 | GCGGACCCCGCCGCCT |
| 258 | GCCTGCGGACCCCGCC |
| 259 | CGACGCCTGCGGACCC |
| 385 | CCCGCGACGCCTGCGG |
| 386 | TCGTCCCGCGACGCCT |
| 387 | CTCCTCGTCCCGCGAC |
| 388 | CGATCTCCTCGTCCCG |
| 389 | GCTCCGATCTCCTCGT |
| 390 | CCCGGCTCCGATCTCC |
| 391 | GTCTCCCGGCTCCGAT |
| 260 | GCGAGTCTCCCGGCTC |
| 5 | CTGCGCGAGTCTCCCG |
| 392 | GGCGCTGCGCGAGTCT |
| 393 | CCATGGCGCTGCGCGA |
| 394 | GGGGCCATGGCGCTGC |
| 395 | AATGGGGGCCATGGCG |
| 396 | TTTAACTGCTTATTTCTTCA |
| 397 | TTGGAGGGGCGACTTATTTT |
| 398 | CTGTGGGATGGGGGCGTGTT |
| 399 | TGGTGGGAAGCTGCGGAGCG |
| 400 | AAAGGAACTGAGGCGGGCGG |
| 401 | GCTGGGGAGGCAACAGACGC |
| 6 | TGCCAGCCAGGGCGTGCAGG |
| 7 | CGAGAATGCAGCGGCAACAG |
| 402 | GAAGAGGGCATCACTGAACA |
| 403 | TTTTCTTTTGTTTCAAACAA |
| 261 | TTAAAAAACACAATGCATTA |
| 404 | ACATGTATAAGATACTCTTT |
| 405 | TGAGCTTCTCTTTTTAGGAT |
| 406 | TGCTGTGCACCAATTGCACA |
| 407 | AACAGTCCAGAAATTTCTCC |
| 408 | AGAAGGCGTCCATTCATCCT |
| 409 | ACAAATCTTAAATAACGGGG |
| 410 | CCAGGGTTATGTACAAGGTC |
| 9 | CCAAGCAATGTGCACGTCAC |
| 411 | AAATTTCTACCGTTCCATAC |
| 412 | ACAAGGTTTTAAAAACACCC |
| 413 | AAGGACAGGAACAACCCCAA |
| 414 | ACATCTCTATGATTCTCAAC |
| 415 | GAAATACTCCAAGAACACAG |
| 416 | AGCAGATTAGTCCTCAGTGT |
| 417 | GTAGGGACTGGAATGAAGAT |
| 418 | ATGAGAGCAGGCACTGAGGG |
| 419 | TCACCTCCCAGGTTATTTGG |
| 420 | CTCCTGAGATATCCTGATTG |
| 421 | AGGTCTGTTCCACCTTGGAC |
| 422 | AGACGCTGGGAAAGGCAAAG |
| 423 | GCTGCACTACCGGGGGTATG |
| 424 | CACCCCAGCCTCCACCCACA |
| 425 | CTGGCCTGACTTCGTGCAGA |
| 426 | ACAGGCTGTGGAGGAGGACG |
| 427 | AGGCTGGGGAGGGGGCAGTG |
| 262 | GATCACAGCACTGTGGACAC |
| 263 | GACTGGAGGACTTCCCTCGG |
| 264 | TGTCAGGGCACTGTGACTTA |
| 265 | GCGGGAGTTTGCTTCTCACC |
| 10 | CAAAGAGATGGAGGCTTCCA |
| 428 | CTCCAGACTAACTGTTTTTC |
| 429 | AGAAGGGCCTGGGCCACAGG |
| 430 | TTGGGATGATGCCTGGGGAC |
| 431 | GGACTAGGGAAAATGAGCTG |
| 432 | CCTGACCCTTGAACGAAGGC |
| 433 | CCCATCTGTTCTGGTCCATT |
| 434 | CTGTTCAGGGGCCTCCAGAA |
| 435 | TTCTCCACAGCCATAGCCCT |
| 436 | AGTCCAACGGGCCAAGAACC |
| 437 | GAGGGTACAGGGTCTGTGTG |
| 438 | TGACTGAAGATGCTTGCCGA |
| 439 | ATCTGAAACTGAGGATAATC |
| 440 | CAGCCGCAGCTCTC |
| 441 | TGGGCCAGCCGCAG |
| 442 | CGCGCTGGGCCAGC |
| 443 | CAGCCGCAGCTCTCCG |
| 266 | CGCGCTGGGCCAGCCG |
| 444 | GTGGGCGCGCTGGGCC |
| 445 | CAGCCGCAGCTCTCCGCC |
| 446 | TGGGCCAGCCGCAGCTCT |
| 447 | CGCGCTGGGCCAGCCGCA |
| 448 | GTGGGCGCGCTGGGCCAG |
| 449 | CAGCTCTCCGCCCC |
| 450 | GGGCCAGCCGCAGC |
| 451 | CAGCTCTCCGCCCCGC |
| 452 | AGCCGCAGCTCTCCGC |
| 453 | GGGCCAGCCGCAGCTC |
| 454 | CAGCTCTCCGCCCCGCAG |
| 455 | AGCCGCAGCTCTCCGCCC |
| 456 | GGGCCAGCCGCAGCTCTC |
| 457 | GCGCTGGGCCAGCCGCAG |
| 458 | CGTCCCGCGACGCC |
| 459 | CTCCTCGTCCCGCG |
| 460 | CCGATCTCCTCGTC |
| 461 | CGTCCCGCGACGCCTG |
| 462 | CCGATCTCCTCGTCCC |
| 463 | CGGCTCCGATCTCCTC |
| 464 | CGTCCCGCGACGCCTGCG |
| 465 | CTCCTCGTCCCGCGACGC |
| 466 | CCGATCTCCTCGTCCCGC |
| 467 | CGGCTCCGATCTCCTCGT |
| 468 | GATCTCCTCGTCCC |
| 469 | GCTCCGATCTCCTC |
| 470 | TCCCGGCTCCGATC |
| 471 | GATCTCCTCGTCCCGC |
| 472 | TCCCGGCTCCGATCTC |
| 473 | AACACAAGGTATTATGAAGT |
| 474 | GATGATGTATGACCCCACAC |
| 475 | CCATCTTCTCTTACAAACAC |
| 476 | TTATACAGAGAATAAAATGA |
| 477 | TTCTGCTTTAGAGCTAAGTT |
| 478 | CCTGCATTTGCTGCTTTAGT |
| 479 | GAGGATGGCGAGACAGCCTT |
| 480 | AATGAGAGCTGCTGAGTCTT |
| 481 | TGGTGTGCTCACCACTGGAG |
| 482 | GACAACAGCAGCAGCACAAA |
| 483 | CCACTGTTATTATATTTCAC |
| 484 | AGGGGACATTTTTGTGACTT |
| 485 | GGGCGGCGAGGGGGCTGGGC |
| 486 | CATGGCCTGCAGGAGGTCAA |
| 487 | CACTAGACAAGTAATACACA |
| 488 | ACAGCACTTTGAGAGGACAT |
| 489 | AGGTGGCGCCGAGCTCGCGT |
| 490 | AGGCTCTGAAAGGGAGGCGG |
| 491 | GAGCAGAGAGGGCGGGGAGC |
| 492 | AGAGAACACCACAATGCAGC |
| 493 | GAGATTTCAAAGCCTTGAGA |
| 494 | GACTAATCTCAGTGCAAGGG |
| 495 | GAGACGGGGAGAGATCTGAC |
| 496 | AGGTCGTATAAGTTGGGAGG |
| 497 | GGTTCCGTCCTAAGGAAATC |
| 498 | CGCCCGGCGCAGGTGCCTGC |
| 499 | AACAAGCAGCGGGAGTAAGA |
| 500 | TGGTCCGAGGGAGGGGACAG |
| 501 | CCTGAAGCATGAGCACTGTT |
| 502 | ACATCTTCGACAAACAAGGT |
| 503 | TAACAGAGAAAGGGAAACCA |
| 504 | ATAAATTATTTTTATATAAA |
| 505 | TTTTTTAAAATATCCTTTTG |
| 506 | AAGTTTCAAGACAGACTAGC |
| 507 | CTGATAATTTTAAGGTAAAC |
| 508 | TACTTTCAAACACTGAGATT |
| 509 | GATATATGTTTGTGCTTCAG |
| 510 | GTACAGAATGGTACAGAGAT |
| 511 | TATTAGACTCAAGTGCTTTA |
| 512 | GGGGTGCTGATTTCTTTATT |
| 513 | AGCCCCCTGGACACCGGGAA |
| 514 | TGATTTCTTTATTTATTAGA |
| 515 | GGGGTGCTGATTTCTTTATT |
| 516 | AGGGGTGCTGATTTCTTTAT |
| 517 | CGGGAAGGGGTGCTGATTTC |
| 518 | AATTATTTTTATATAAATAA |
| 519 | TTTGATAAATTATTTTTATA |
| 520 | TCCTTTTGATAAATTATTTT |
| 521 | AAATATCCTTTTGATAAATT |
| 522 | GAGTCTCCCGGCTCCG |
| 267 | GATCTCCTCGTCCCGCGA |
| 523 | GCTCCGATCTCCTCGTCC |
| 524 | TCCCGGCTCCGATCTCCT |
| 525 | GAGTCTCCCGGCTCCGAT |
| 526 | CCCCTTCATTCTCTAG |
| 527 | TCCCACCACTTTGGGA |
| 528 | ACTCCTGTAATCCCAC |
| 268 | GCAGTGGCTCACTCCT |
| 529 | TGTGCCAAGTGCAGTG |
| 530 | GTCCTGCCTCAGCCAC |
| 531 | TCAAGTGATTGTCCTG |
| 532 | ACTCCCGGGTTCAAGT |
| 533 | GCAACCTCCAACTCCC |
| 534 | GAGTGCAGTGGCACAA |
| 535 | CGCCCAGGCTGGAGTG |
| 536 | TCTTGCTCTGTCGCCC |
| 269 | TTTTGAGACAGTCTTG |
| 537 | GTGTGCCAAGTGCAGT |
| 538 | GTAGAGACAGGGTTTC |
| 539 | ACGCCCGGCTAATTTT |
| 540 | TGTAATCCTAGCACTT |
| 541 | GGCTCATGCCTGTAAT |
| 270 | CGGGCACGGTGGCTCA |
| 542 | ATCTGTATCTTAAATG |
| 543 | CCCGGCCTGGATCTGT |
| 544 | AGCCACCATGCCCGGC |
| 545 | GAGATGGAGTTTTACT |
| 546 | TTTATTTTTTGAGATG |
| 547 | GAAGTGAACACAAGTT |
| 548 | GAAAAAGAGTGAAGTG |
| 549 | CTGTGGAGGAGGACGC |
| 550 | ACGAAGGCGGACTAGG |
| 551 | TCCATTCCTGACCCTT |
| 552 | AGAACCCATCTGTTCT |
| 553 | CTCTGTTCAGGGGCCT |
| 554 | GTCCTGCCTCAGCCAC |
| 555 | GACAGTCTTGCTCTGT |
| 556 | TTCTTTTTGAGACAGT |
| 557 | CTCCTGTACTTAAGGT |
| 558 | GTTTTGAGACGGAGTC |
| 559 | TTGTTTGTTTGTTTTG |
| 560 | GTTTGTTTGTTTGTTT |
| 561 | GCCATTTACAGGACTG |
| 562 | AGCCGGGTGTCGTGGC |
| 563 | ACAAAAAATTAGCCGG |
| 564 | CCAGGTGCAGTGGCTC |
| 565 | GGCACCATCACCCATG |
| 566 | ACACCCAAAGCCTGGC |
| 567 | GGGGAAGATTTACACC |
| 568 | CTAAAGATGAAGGGGA |
| 569 | TCAAGTCAGGTCTAAA |
| 570 | GAGGATGGGGGTGCGG |
| 571 | TAACGGGGGTGAGGAT |
| 572 | GAGGGGAAGGTAACGG |
| 573 | AGAGGGGACTGAGGGG |
| 574 | GAGGGACTGCAGGAAA |
| 575 | TGGGGAGGAGGAGGGA |
| 576 | CCCAGGAGGGTGGGGA |
| 577 | CAAGGAATAAAGACGA |
| 578 | AGTCTGGGCGCTCAAG |
| 579 | AAAAGGGAGCGGAGTC |
| 580 | AGGGACTGCAGGAAAA |
| 581 | TAGAGATGGGGTTTCA |
| 582 | GTATTTTTAGTAGAGA |
| 583 | ATTAATTTTTGTATTT |
| 584 | ACCACGCCCAATTAAT |
| 585 | AGAGACTGGGTTTTAC |
| 586 | TATTTTTAGTAGAGAC |
| 587 | CTAATTTTTGTATTTT |
| 588 | TGAACAAAGGTACGTCA |
| 589 | GCCAGCTGACTGCTTGA |
| 590 | TTTCTTTTCTGTAATAA |
| 591 | AGAGTGGAAGAGTTTCT |
| 592 | GAAATAAAGAAAAAGAG |
| 593 | ATAAACTAAAAAGGAAG |
| 594 | GGTACGTCACAGGAACAT |
| 1 | CAGAGGCCAGCTGACTGC |
| 595 | GAGTTTCTTTTCTGTAAT |
| 596 | AATAAAGAAAAAGAGTGA |
| 597 | TACATTTTATGCAGCCAAT |
| 598 | CCAACTTAGGCCTACATTT |
| 599 | AGAAAGCCCTTCAGAGTGG |
| 600 | TAATCAAAGAAAGCCCTTC |
| 601 | TAAAAAGGAAGAAATAAAG |
| 602 | AAAGAAAAAGAGTGACGGAA |
| 603 | AAGGAAGAAATAAAGAAAAA |
| 604 | TGCAGCCAATGAGGTATGAGA |
| 605 | CTTTTCTGTAATAAGCTGCTT |
| 606 | GTGGAAGAGTTTCTTTTCTGT |
| 607 | CTTATTATTCACCAAAAGTT |
| 608 | TGCTCCTTATTATTCACCAA |
| 609 | CCTTATTATTCACCAAAAGT |
| 610 | CTGCTCCTTATTATTCACCA |
| 611 | CTCCTTATTATTCACCAAAA |
| 612 | TTACCATGTGATCCAGCAAT |
| 613 | AATCCTGCTCCTTATTATTC |
| 614 | GGTTGCTGGAAGAATCTAAA |
| 615 | TTGCTGGAAGAATCTAAAAT |
| 616 | GTTGCTGGAAGAATCTAAAA |
| 617 | AGGTTGCTGGAAGAATCTAA |
| 618 | AATCAAAGGAGATAATAATG |
| 619 | CAGGTTGCTGGAAGAATCTA |
| 620 | GCTGGAAGAATCTAAAATCA |
| 621 | CAAATTAAAACAACAATACC |
| 622 | GTTGGACAGGCTGGTCTCAA |
| 623 | GACAAAAACAACAACAAAGA |
| 624 | GTAGGAGTTTCCATTTTTCT |
| 625 | TTCCACTTGGGGAACCTATG |
| 626 | AGTTTCCATTTTTCTACCTC |

In one embodiment, the ASO provided herein comprises a base sequence selected from SEQ ID NOs: 2, 17, 75, 168, 188, 197 and 225, optionally wherein the ASO has one or more modified bases, one or more modified sugars, and/or one or more modified internucleoside linkages. In another embodiment, the ASO provided herein has all 2′-MOE sugars and all phosphorothioate internucleoside linkages, and has a base sequence selected from SEQ ID NOs: 2, 17, 75, 168, 188, 197 and 225.

### III. Synthesis of the Antisense Oligonucleotides

The ASOs provided herein may be prepared according to standard methods well known in the art. See, e.g., Beaucage, et al. Tetrahedron 1992, 48(12), 2223; Reese Org. & Biomol. Chem. 2005, 3(21), 3851-3868.

### IV. Pharmaceutical Compositions

The pharmaceutical compositions provided herein contain therapeutically effective amounts of one or more of the ASOs provided herein and a pharmaceutically acceptable carrier, diluent or excipient.

The ASOs can be formulated into suitable pharmaceutical preparations such as solutions, suspensions, in sterile solutions or suspensions for ophthalmic or parenteral administration, as well as transdermal patch preparation. Typically, the ASOs described above are formulated into pharmaceutical compositions using techniques and procedures well known in the art (see, e.g., Ansel Introduction to Pharmaceutical Dosage Forms, Twelfth Edition 2021).

In the compositions, effective concentrations of one or more ASOs or pharmaceutically acceptable salts is (are) mixed with a suitable pharmaceutical carrier or vehicle. In certain embodiments, the concentrations of the ASOs in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of ASO is dissolved, suspended, dispersed or otherwise mixed in a selected vehicle at an effective concentration such that the treated condition is relieved or ameliorated. Pharmaceutical carriers or vehicles suitable for administration of the ASOs provided herein include any such carriers known to those skilled in the art to be suitable for the particular mode of administration.

In addition, the ASOs may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of an ASO provided herein in phosphate buffered saline lacking divalent cations (PBS) is added and the flask shaken until the lipid film is dispersed. The resulting vesicles are washed to remove unencapsulated ASO, pelleted by centrifugation, and then resuspended in PBS.

The active ASO is included in the pharmaceutically acceptable carrier in an amount sufficient to exert a therapeutically useful effect in the absence of undesirable side effects on the subject treated. The therapeutically effective concentration may be determined empirically by testing the ASOs in in vitro and in vivo systems described herein and then extrapolated therefrom for dosages for humans. In some embodiments, the active ASO is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active ASO in specific subjects or subject populations.

The concentration of active ASO in the pharmaceutical composition will depend on tissue distribution, inactivation and excretion rates of the active ASO, the physicochemical characteristics of the ASO, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of ASO per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the ASOs described herein or pharmaceutically acceptable salts thereof are mixed with a suitable pharmaceutical carrier or vehicle for systemic, topical or local administration to form pharmaceutical compositions. ASOs are included in an amount effective for ameliorating one or more symptoms of, or for treating, retarding progression, or preventing. The concentration of active ASO in the composition will depend on absorption, tissue distribution, inactivation, excretion rates of the active ASO, the dosage schedule, amount administered, particular formulation as well as other factors known to those of skill in the art.

The compositions are intended to be administered by a suitable route, including but not limited to parenteral, subcutaneous, intravenous, intramuscular, intraperitoneal, intrathecal, intracerebroventricular, intraocular, mucosal, dermal, transdermal, buccal, rectal, topical or local. The compositions are in liquid, semi-liquid or solid form and are formulated in a manner suitable for each route of administration. The term “intracerebroventricular” refers to administration of a composition into the ventricular system of the brain, e.g., via injection, infusion, or implantation (for example, into a ventricle of the brain). The term “intraocular” refers to the administration of a composition to the eye region, e.g., via injection, infusion, or implantation (for example, into the eyeball) or topical/ophthalmic administration (for example, using a cream, ointment, gel or liquid drops). The term “intrathecal” refers to administration of a composition into the lumbar region, e.g., via injection, infusion, or implantation (for example, into the subarachnoid space of the spinal cord).

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the ASOs exhibit insufficient solubility, methods for solubilizing ASOs may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the ASO(s), the resulting mixture may be a solution, suspension, emulsion or the like. The form of the resulting mixture depends upon a number of factors, including the intended mode of administration and the solubility of the ASO in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as powders, granules, sterile parenteral solutions or suspensions, and oil water emulsions containing suitable quantities of the ASOs or pharmaceutically acceptable salts thereof. The pharmaceutically therapeutically active ASOs and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active ASO sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the ASO provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated ASO remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active ASOs or pharmaceutically acceptable salts may be prepared with carriers that protect the ASO against rapid elimination from the body, such as time release formulations or coatings.

The compositions may include other active ASOs to obtain desired combinations of properties. The ASOs provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to STXBP1 haploinsufficiency. It is to be understood that such combination therapy constitutes a further aspect of the compositions and methods of treatment provided herein.

Further encompassed are anhydrous pharmaceutical compositions and dosage forms containing an ASO provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some ASOs. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### A. Injectables, Solutions and Emulsions

Parenteral administration, generally characterized by injection, either intrathecally, intracerebroventricularly, intraocularly, subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. For intrathecal or intracerebroventricular delivery, one or more preservatives, stabilizers or excipients may be used in the composition. In this regard, numerous well known and routinely employed preservatives, stabilizers and excipients useful for formulations for intrathecal or intracerebroventricular delivery are known in the art. More specifically, examples of such additives to formulations for use in intrathecal or intracerebroventricular delivery are described in WO 2013/096899. Implantation of a slow release or sustained release system, such that a constant level of dosage is maintained is also contemplated herein. Briefly, an ASO provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The ASO diffuses through the outer polymeric membrane in a release rate controlling step. The percentage of active ASO contained in such parenteral compositions is highly dependent on the specific nature thereof, as well as the activity of the ASO and the needs of the subject.

Parenteral administration of the compositions includes intrathecal, intracerebroventricular, intraocular, intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active ASO is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active ASO is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active ASO to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The ASO may be suspended in micronized or other suitable form or may be derivatized to produce a more soluble active product or to produce a prodrug. The form of the resulting mixture depends upon a number of factors, including the intended mode of administration and the solubility of the ASO in the selected carrier or vehicle. The effective concentration is sufficient for ameliorating the symptoms of the condition and may be empirically determined.

### B. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving an ASO provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the ASO. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected ASO. Such amount can be empirically determined.

### C. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The ASOs or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The ASOs may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active ASO alone or in combination with other pharmaceutically acceptable excipients can also be administered.

These solutions, particularly those intended for ophthalmic use, may be formulated as 0.01%-10% isotonic solutions, pH about 5-7, with appropriate salts.

### D. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or ASOs.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### E. Targeted Formulations

The ASOs provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of an ASO provided herein in phosphate buffered saline lacking divalent cations (PBS) is added and the flask shaken until the lipid film is dispersed. The resulting vesicles are washed to remove unencapsulated ASO, pelleted by centrifugation, and then resuspended in PBS.

### V. Dosing

The ASOs and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the ASO is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the ASO provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the ASO can be administered in an amount of about 25 mg/day. In a particular embodiment, the ASO can be administered in an amount of about 10 mg/day. In a particular embodiment, the ASO can be administered in an amount of about 5 mg/day. In a particular embodiment, the ASO can be administered in an amount of about 4 mg/day. In a particular embodiment, the ASO can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the ASO administered is sufficient to provide a plasma concentration of the ASO at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the ASO administered is sufficient to provide a plasma concentration of the ASO at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of an ASO provided herein, or a derivative thereof. Once steady state is reached, there are minor peaks and troughs on the time dependent curve of the plasma concentration of the ASO.

In certain embodiments, the amount of the ASO administered is sufficient to provide a maximum plasma concentration (peak concentration) of the ASO, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the ASO administered is sufficient to provide a minimum plasma concentration (trough concentration) of the ASO, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the ASO administered is sufficient to provide an area under the curve (AUC) of the ASO, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the ASO provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The ASO provided herein, or a derivative thereof, may be formulated, alone or together, in suitable dosage unit with pharmaceutically acceptable excipients, carriers, adjuvants and vehicles, appropriate for each route of administration.

In one embodiment, the ASO provided herein, or a derivative thereof, is administered orally. In another embodiment, the ASO provided herein, or a derivative thereof, is administered parenterally. In yet another embodiment, the ASO provided herein, or a derivative thereof, is administered intravenously.

The ASO provided herein, or a derivative thereof, can be delivered as a single dose such as, e.g., a single bolus injection, or oral tablets or pills; or over time, such as, e.g., continuous infusion over time or divided bolus doses over time. The ASO can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The ASO provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic ASO, such as the ASO provided herein, or a derivative thereof, is administered once or more than once each day, for example, for a period of time. The term “continuous” is intended to mean that a therapeutic ASO, such as the ASO provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the ASO provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic ASO, such as the ASO provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the ASO provided herein, or a derivative thereof, is administered once a day. In another embodiment, the ASO provided herein, or a derivative thereof, is administered twice a day. In yet another embodiment, the ASO provided herein, or a derivative thereof, is administered three times a day. In still another embodiment, the ASO provided herein, or a derivative thereof, is administered four times a day.

In certain embodiments, the ASO provided herein, or a derivative thereof, is administered once per day from one day to six months, from one week to three months, from one week to four weeks, from one week to three weeks, or from one week to two weeks. In certain embodiments, the ASO provided herein, or a derivative thereof, is administered once per day for one week, two weeks, three weeks, or four weeks. In one embodiment, the ASO provided herein, or a derivative thereof, is administered once per day for 4 days. In one embodiment, the ASO provided herein, or a derivative thereof, is administered once per day for 5 days. In one embodiment, the ASO provided herein, or a derivative thereof, is administered once per day for 6 days. In one embodiment, the ASO provided herein, or a derivative thereof, is administered once per day for one week. In another embodiment, the ASO provided herein, or a derivative thereof, is administered once per day for two weeks. In yet another embodiment, the ASO provided herein, or a derivative thereof, is administered once per day for three weeks. In still another embodiment, the ASO provided herein, or a derivative thereof, is administered once per day for four weeks.

### VI. Methods of Treatment

In one embodiment, the ASOs provided herein are useful for increasing expression of STXBP1. In another embodiment the ASOs provided herein increase the expression of STXBP1 protein when contacted with a cell such as a neuronal cell. In another embodiment, an ASO provided herein increases a level of functional STXBP1 protein in a neuronal cell relative to a control cell not contacted with the ASO. In another embodiment, an ASO provided herein increases the synaptic local expression of STXBP1 protein in a composition containing neuronal cells. In one embodiment, the composition containing neuronal cells is in vivo.

In one embodiment, the ASOs provided herein modulate expression of STXBP1. In another embodiment, the ASOs provided herein increase expression of STXBP1 in a neuronal cell. In another embodiment, the neuronal cell is haploinsufficient for STXBP1 and/or heterozygous for a deleterious mutation in STXBP1.

Also provided is a method of treating, preventing, or delaying the onset of an STXBP1 disorder using the ASOs provided herein. In one embodiment, the methods provided herein result in increased levels of STXBP1 in a cell and therefore are useful in methods of treatment of diseases which are associated with STXBP1 defects (i.e., reduced STXBP1 levels and/or loss-of-function mutations of the STXBP1 gene). In another embodiment, provided are methods of treating, preventing, or delaying the onset of diseases caused by a quantitative decrease in the predetermined, normal STXBP1 protein level. In certain embodiments, the methods provided herein can be performed in vitro, ex vivo or in vivo.

In one embodiment, the STXBP1 disorder is associated with STXBP1 haploinsufficiency. In one embodiment, the STXBP1 disorder is encephalopathy. In another embodiment, the STXBP1 disorder is STXBP1 encephalopathy. In another embodiment, the STXBP1 disorder is epilepsy. In another embodiment, the STXBP1 disorder is epileptic encephalopathy.

In another embodiment, the STXBP1 disorder is a severe early onset epileptic encephalopathy or a non-syndromic epilepsy selected from Ohtahara syndrome, West syndrome, Lennox-Gastaut syndrome, Dravet syndrome, early myoclonic encephalopathy, an unclassified early onset epileptic encephalopathy that is associated with STXBP1 haploinsufficiency, atypical Rett syndrome and a severe intellectual disability without epilepsy associated with STXBP1 haploinsufficiency. In certain embodiments, the STXBP1 disorder is epilepsy, global delay, cognitive impairment (mild to profound), a movement disorder, hypotonia or autism.

In another embodiment, the STXBP1 disorder is DEE4, Developmental and epileptic encephalopathy 4, Developmental and epileptic encephalopathy, type 4, Early-infantile epileptic encephalopathy 4, EIEE4, STXBP1 encephalopathy with epilepsy, STXBP1 epileptic encephalopathy, STXBP1-related developmental and epileptic encephalopathy, STXBP1-related early-onset encephalopathy, or STXBP1-related epileptic encephalopathy.

In another embodiment, provided is a method for treating, preventing or delaying the onset of a disorder caused by an STXBP1 mutation using an ASO provided herein. Many such mutations, many of which are missense mutations, are known (see, e.g., www.stxbp1disorders.org/what-is-stxbp1).

In another embodiment, provided is a method for treating, preventing, or delaying the onset of a disease in which disease processes lower STXBP1 levels using an ASO provided herein. In another embodiment, provided is a method of increasing STXBP1 levels in a subject having a disease in which disease processes lower STXBP1 levels by administering to the subject an ASO provided herein. In another embodiment, the disease is amyotrophic lateral sclerosis (ALS). In these embodiments, the STXBP1 gene may not carry a mutation.

### VII. Combination Therapy with a Second Active Agent

The ASO provided herein, or a derivative thereof, can also be combined or used in combination with other therapeutic agents useful in the treatment and/or prevention of an STXBP1 disorder.

In one embodiment, provided herein is a method of treating, preventing, or delaying an STXBP1 disorder, comprising administering to a subject an ASO provided herein, or a derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as an ASO provided herein, an ASO provided herein, e.g., the ASO provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the ASO provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the ASO provided herein, or a derivative thereof, is independent of the route of administration of a second therapy. In another embodiment, the ASO provided herein, or a derivative thereof, is administered intrathecally, intracerebroventricularly, intraocularly or intravenously. Thus, in accordance with these embodiments, the ASO provided herein, or a derivative thereof, is administered intrathecally, intracerebroventricularly, intraocularly or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, intracerebroventricularly, or in a slow release dosage form. In one embodiment, the ASO provided herein, or a derivative thereof, and a second therapy are administered by the same mode of administration, e.g., intrathecally, intracerebroventricularly, intraocularly or intravenously. In another embodiment, the ASO provided herein, or a derivative thereof, is administered by one mode of administration, e.g., intrathecally, intracerebroventricularly, intraocularly or intravenously, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the ASO provided herein, or a derivative thereof, and any optional additional active agents concurrently administered to the subject.

One or more second active ingredients or agents can be used together with the ASO provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or synthetic or recombinant proteins.

In one embodiment, the second active agent is stiripentol, cannabidiol, levetiracetam, a gene therapy, 4-phenylbutyrate, adrenocorticotropic hormone (ACTH), topiramate or prednisone.

### VIII. Examples

The examples below are meant to illustrate certain embodiments provided herein, and not to limit the scope of this disclosure.

### Example 1

ASOs with varying lengths, were designed to the STXBP1 mRNA sequence and then tested in STXBP1 heterozygous iPSC-derived neurons cell line model for resuce of the STXBP1 protein levels.

### AI-Based ASO Design

An artificial intelligent (AI)-based platform developed by Deep Genomics, Inc. (Toronto, Canada) was used to design ASOs that target STXBP1 mRNA or pre-mRNA and predicted to rescue or increase STXBP1 expression using a variety of different mechanisms. The set of 390 ASOs identified in Table 3 were then selected for primary screening in vitro using an STXBP1 haploinsufficient cell model with a HiBiT-Tag Luciferase System as set out below.

### STXBP1 Cell Model

iPSC-derived neurons (iNeurons) obtained under licence from iPS Academia Japan (Kyoto, Japan) were used as a physiologically-relevant model. As iNeurons mature in culture, they form functional pre- and postsynaptic specializations, and by 3 weeks in vitro, they exhibit action potentials, voltage-gated Na+ and K+ currents and evoke excitatory postsynaptic currents (Zhang et al., Neuron 78, no. 5 (June 2013): 785-98; Meijer et al., Cell Reports 27, no. 7 (May 2019): 2199-2211.e6). Calcium imaging and multi-electrode array recordings have also shown spontaneous and synaptically-mediated action potentials that are blocked by glutamate receptor antagonist CNQX.

The model used has a neurogenin-2 (NGN2) transgene under the control of an inducible promoter. iPSCs with this NGN2 inducible system robustly express neurogenin-2, which rapidly and irreversibly induces their differentiation into excitatory iNeurons.

### STXBP1 Haploinsufficiency

The pathogenic mechanism of STXBP1 mutations is consistent with haploinsufficiency (Stamberger et al., supra). Therefore, this was modeled by introducing a frameshift mutation near the 5′ end of the STXBP1 transcript.

### CRISPR-Cas9 Editing

Starting with NGN2-inducible iPSCs as described above, CRISPR-Cas9 was used to edit cells with a guide RNA (5′-TGGTGGATCAGTTAAGCATG-3′) (SEQ ID NO: 627) that cuts specifically and efficiently at position chr9:127,653,738 (hg38). One successfully edited clone (1A2) showed a 2 nucleotide deletion on one allele, leading to a disruption in the reading frame to make the STXBP1 Het-HiBiT iNeurons (i.e., STXBP1 heterozygous clones).

### Protein Quantification of STXBP1 Heterozygous Clones

Disruption of the reading frame leading to a decrease in STXBP1 protein was confirmed in 2 independent batches of iNeurons. Protein lysates were collected 7 days after neuronal induction and STXBP1 protein levels were measured using the Jess WB system. STXBP1 signals were normalized to a corresponding gamma-tubulin signal as a loading control. Clone 1A2 exhibited an approximately 50% reduction in STXBP1 protein.

### HiBiT-TagLuciferase System

A HiBiT tag was introduced at aa527 of the STXBP1 protein of clone 1A2 (524-531 loop). The resulting clone 1A2-E11 was tested for protein stability using the Jess WB system. STXBP1 Heterozygous HiBiT-tagged iNeurons showed approximately half the amount of STXBP1 protein when compared to untagged WT iNeurons, indicating that protein stability was not adversely affected by the HiBiT tag.

### HiBiT-Tag Screening

STXBP1 Het-HiBiT iNeurons (clone 1A2-E11) were seeded into pre-coated PDL 96-well plates at a density of 25,000 cells/well. On the day after plating, media was removed and ASOs were added at a final concentration of 5 μM. After 10 days, a Cell-Titer Fluor assay was used to estimate the number of viable cells and a HiBiT assay was conducted.

HiBiT values were normalized to CTF values using a linear regression. Fold change values were calculated relative to the average of non-targeting controls. Data from the HiBiT-tag assay are provided in Table 4 below showing the fold change over a non-targeting control.

TABLE 4
HiBiT assay data presented in Average Fold Change

| SEQ ID NO: | HiBiT Average Fold Change |
|---|---|
| 1 | 1.89 |
| 2 | 2.15 |
| 3 | 2.8 |
| 4 | 2.62 |
| 5 | 2.11 |
| 6 | 2.65 |
| 7 | 1.74 |
| 8 | 3.11 |
| 9 | 1.73 |
| 10 | 2.02 |
| 11 | 1.28 |
| 12 | 1.44 |
| 13 | 1.17 |
| 14 | 1.28 |
| 15 | 1.2 |
| 16 | 1.35 |
| 17 | 1.49 |
| 18 | 1.51 |
| 19 | 1.53 |
| 20 | 1.34 |
| 21 | 1.38 |
| 22 | 1.19 |
| 23 | 1.36 |
| 24 | 1.28 |
| 25 | 1.45 |
| 26 | 1.3 |
| 27 | 1.35 |
| 28 | 1.26 |
| 29 | 1.25 |
| 30 | 1.3 |
| 31 | 1.28 |
| 32 | 1.3 |
| 33 | 1.49 |
| 34 | 1.1 |
| 35 | 0.92 |
| 36 | 1.01 |
| 37 | 1.03 |
| 38 | 0.96 |
| 39 | 0.97 |
| 40 | 0.89 |
| 41 | 0.95 |
| 42 | 1.06 |
| 43 | 1.09 |
| 44 | 1.07 |
| 45 | 1 |
| 46 | 1.27 |
| 47 | 1.23 |
| 48 | 1.11 |
| 49 | 1.28 |
| 50 | 1.03 |
| 51 | 1.23 |
| 52 | 1.15 |
| 53 | 0.96 |
| 54 | 1.13 |
| 55 | 1.09 |
| 56 | 0.95 |
| 57 | 2.19 |
| 58 | 1.08 |
| 59 | 1.62 |
| 60 | 2.11 |
| 61 | 2.24 |
| 62 | 1.98 |
| 63 | 0.97 |
| 64 | 1.07 |
| 65 | 1.3 |
| 66 | 2.25 |
| 67 | 1.87 |
| 68 | 1.68 |
| 69 | 1.2 |
| 70 | 1.8 |
| 71 | 0.99 |
| 72 | 1.02 |
| 73 | 1.81 |
| 74 | 1.83 |
| 75 | 2.94 |
| 76 | 2.1 |
| 77 | 1.9 |
| 78 | 1.34 |
| 79 | 1.21 |
| 80 | 0.95 |
| 81 | 1.14 |
| 82 | 1.34 |
| 83 | 1.53 |
| 84 | 1.89 |
| 85 | 2.04 |
| 86 | 1.63 |
| 87 | 1.39 |
| 88 | 1.22 |
| 89 | 1.61 |
| 90 | 1.47 |
| 91 | 1.58 |
| 92 | 1.4 |
| 93 | 1.31 |
| 94 | 1.59 |
| 95 | 1.5 |
| 96 | 1.25 |
| 97 | 1.21 |
| 98 | 1.18 |
| 99 | 1.64 |
| 100 | 1.92 |
| 101 | 1.59 |
| 102 | 1.66 |
| 103 | 1.4 |
| 104 | 1.86 |
| 105 | 1.19 |
| 106 | 1.52 |
| 107 | 1.06 |
| 108 | 1.22 |
| 109 | 1.51 |
| 110 | 1.53 |
| 111 | 1.91 |
| 112 | 1.37 |
| 113 | 1.16 |
| 114 | 1.79 |
| 115 | 0.64 |
| 116 | 1.22 |
| 117 | 1.28 |
| 118 | 0.67 |
| 119 | 1.43 |
| 120 | 1.56 |
| 121 | 1.17 |
| 122 | 1.33 |
| 123 | 1.38 |
| 124 | 1.5 |
| 125 | 1.43 |
| 126 | 1.43 |
| 127 | 1.26 |
| 128 | 1.5 |
| 129 | 1.56 |
| 130 | 1.1 |
| 131 | 1.58 |
| 132 | 1.13 |
| 133 | 1.17 |
| 134 | 1.3 |
| 135 | 1.08 |
| 136 | 1.25 |
| 137 | 1.01 |
| 138 | 0.93 |
| 139 | 1.22 |
| 140 | 1.03 |
| 141 | 1.19 |
| 142 | 1.41 |
| 143 | 1.17 |
| 144 | 1.16 |
| 145 | 1.23 |
| 146 | 1.15 |
| 147 | 0.99 |
| 148 | 1 |
| 149 | 1.04 |
| 150 | 0.98 |
| 151 | 1.1 |
| 152 | 1.1 |
| 153 | 1.28 |
| 154 | 1.04 |
| 155 | 1.07 |
| 156 | 0.94 |
| 157 | 1.06 |
| 158 | 1.08 |
| 159 | 0.98 |
| 160 | 1.08 |
| 161 | 1.11 |
| 162 | 0.91 |
| 163 | 0.9 |
| 164 | 1.18 |
| 165 | 1.13 |
| 166 | 0.97 |
| 167 | 1 |
| 168 | 1.19 |
| 169 | 1.11 |
| 170 | 1.06 |
| 171 | 2.23 |
| 172 | 2.22 |
| 173 | 1.99 |
| 174 | 1.47 |
| 175 | 1.34 |
| 176 | 1.15 |
| 177 | 2.01 |
| 178 | 2.22 |
| 179 | 2.03 |
| 180 | 1.93 |
| 181 | 1.35 |
| 182 | 0.93 |
| 183 | 0.96 |
| 184 | 1.47 |
| 185 | 0.84 |
| 186 | 1.3 |
| 187 | 1.38 |
| 188 | 2.36 |
| 189 | 2.27 |
| 190 | 2.56 |
| 191 | 0.92 |
| 192 | 1 |
| 193 | 1.57 |
| 194 | 1.06 |
| 195 | 2.37 |
| 196 | 1.9 |
| 197 | 1.67 |
| 198 | 1.27 |
| 199 | 1.3 |
| 200 | 1.25 |
| 201 | 1.32 |
| 202 | 1.66 |
| 203 | 1.46 |
| 204 | 1.44 |
| 205 | 1.3 |
| 206 | 1.31 |
| 207 | 1.04 |
| 208 | 1.19 |
| 209 | 1.33 |
| 210 | 1.37 |
| 211 | 1.24 |
| 212 | 0.91 |
| 213 | 1.46 |
| 214 | 1.3 |
| 215 | 1.06 |
| 216 | 1.13 |
| 217 | 1.34 |
| 218 | 1.41 |
| 219 | 1.25 |
| 220 | 1.6 |
| 221 | 1.28 |
| 222 | 1.07 |
| 223 | 0.96 |
| 224 | 1.04 |
| 225 | 1.05 |
| 226 | 2.07 |
| 227 | 1.62 |
| 228 | 1.28 |
| 229 | 1.86 |
| 230 | 0.7 |
| 231 | 1.05 |
| 232 | 1.14 |
| 233 | 1.01 |
| 234 | 1.53 |
| 235 | 1.47 |
| 236 | 1.2 |
| 237 | 0.83 |
| 238 | 1.05 |
| 239 | 0.98 |
| 240 | 0.98 |
| 241 | 1.01 |
| 242 | 0.96 |
| 243 | 1.52 |
| 244 | 1.75 |
| 245 | 1.54 |
| 246 | 1.82 |
| 247 | 1.5 |
| 248 | 1.53 |
| 249 | 1.9 |
| 250 | 1.47 |
| 251 | 1.47 |
| 252 | 2.25 |
| 253 | 2.13 |
| 254 | 1.56 |
| 255 | 1.8 |
| 256 | 2.69 |
| 257 | 2.98 |
| 258 | 3.02 |
| 259 | 2.9 |
| 260 | 2.26 |
| 261 | 1.72 |
| 262 | 1.88 |
| 263 | 2.45 |
| 264 | 1.67 |
| 265 | 2.68 |
| 266 | 2.09 |
| 267 | 1.67 |
| 268 | 2.39 |
| 269 | 1.78 |
| 270 | 2.12 |
| 271 | 1.96 |
| 272 | 1.71 |
| 273 | 1.83 |
| 274 | 2.11 |
| 275 | 2.11 |
| 276 | 1.52 |
| 277 | 1.98 |
| 278 | 2.14 |
| 279 | 2.1 |
| 280 | 1.25 |
| 281 | 1.04 |
| 282 | 0.87 |
| 283 | 1.07 |
| 284 | 0.89 |
| 285 | 1.2 |
| 286 | 1.36 |
| 287 | 1.24 |
| 288 | 1 |
| 289 | 1.46 |
| 290 | 1.17 |
| 291 | 1.21 |
| 292 | 0.7 |
| 293 | 1.46 |
| 294 | 0.95 |
| 295 | 0.98 |
| 296 | 0.96 |
| 297 | 1 |
| 298 | 1.46 |
| 299 | 0.9 |
| 300 | 1.2 |
| 301 | 1.21 |
| 302 | 0.94 |
| 303 | 0.89 |
| 304 | 1.08 |
| 305 | 0.71 |
| 306 | 1.07 |
| 307 | 0.91 |
| 308 | 0.92 |
| 309 | 0.85 |
| 310 | 1.03 |
| 311 | 0.86 |
| 312 | 0.8 |
| 313 | 0.81 |
| 314 | 1.05 |
| 315 | 0.81 |
| 316 | 1.34 |
| 317 | 0.91 |
| 318 | 0.7 |
| 319 | 0.88 |
| 320 | 0.91 |
| 321 | 1.08 |
| 322 | 1.29 |
| 323 | 1.32 |
| 324 | 0.95 |
| 325 | 0.92 |
| 326 | 1.2 |
| 327 | 1.35 |
| 328 | 1.08 |
| 329 | 1.01 |
| 330 | 0.5 |
| 331 | 0.86 |
| 332 | 1.33 |
| 333 | 0.96 |
| 334 | 0.87 |
| 335 | 1.29 |
| 336 | 1.44 |
| 337 | 0.93 |
| 338 | 1 |
| 339 | 1.02 |
| 340 | 1.38 |
| 341 | 0.86 |
| 342 | 1.5 |
| 343 | 0.94 |
| 344 | 0.93 |
| 345 | 1.26 |
| 346 | 0.92 |
| 347 | 1.13 |
| 348 | 0.88 |
| 349 | 0.85 |
| 350 | 1.12 |
| 351 | 0.85 |
| 352 | 1.14 |
| 353 | 1.33 |
| 354 | 1.1 |
| 355 | 0.96 |
| 356 | 0.45 |
| 357 | 0.59 |
| 358 | 0.21 |
| 359 | 0.15 |
| 360 | 0.75 |
| 361 | 1.08 |
| 362 | 1.28 |
| 363 | 1.22 |
| 364 | 1.47 |
| 365 | 1.06 |
| 366 | 0.97 |
| 367 | 1.47 |
| 368 | 0.41 |
| 369 | 0.55 |
| 370 | 0.51 |
| 371 | 0.96 |
| 372 | 0.76 |
| 373 | 1 |
| 374 | 1.14 |
| 375 | 1.06 |
| 376 | 1.27 |
| 377 | 0.85 |
| 378 | 1.63 |
| 379 | 0.32 |
| 380 | 1.23 |
| 381 | 1.46 |
| 382 | 1.31 |
| 383 | 0.9 |
| 384 | 1.37 |
| 385 | 1.07 |
| 386 | 1.43 |
| 387 | 1.32 |
| 388 | 0.94 |
| 389 | 1.27 |
| 390 | 0.95 |
| 391 | 1.48 |
| 392 | 1.42 |
| 393 | 1.43 |
| 394 | 0.34 |
| 395 | 0.24 |
| 396 | 0.69 |
| 397 | 0.31 |
| 398 | 0.2 |
| 399 | 0.33 |
| 400 | 0.84 |
| 401 | 0.98 |
| 402 | 1.07 |
| 403 | 1.31 |
| 404 | 1.02 |
| 405 | 1.16 |
| 406 | 1.55 |
| 407 | 3.11 |
| 408 | 1.51 |
| 409 | 0.62 |
| 410 | 0.98 |
| 411 | 0.99 |
| 412 | 1.59 |
| 413 | 1.15 |
| 414 | 0.91 |
| 415 | 0.82 |
| 416 | 1.32 |
| 417 | 0.81 |
| 418 | 0.62 |
| 419 | 1.05 |
| 420 | 1.57 |
| 421 | 0.94 |
| 422 | 1.27 |
| 423 | 0.77 |
| 424 | 0.84 |
| 425 | 0.8 |
| 426 | 0.46 |
| 427 | 0.32 |
| 428 | 1.6 |
| 429 | 1.39 |
| 430 | 0.19 |
| 431 | 0.64 |
| 432 | 1.65 |
| 433 | 0.96 |
| 434 | 1.07 |
| 435 | 1.58 |
| 436 | 1.1 |
| 437 | 0.73 |
| 438 | 1.07 |
| 439 | 1.16 |
| 440 | 1.28 |
| 441 | 0.92 |
| 442 | 1.44 |
| 443 | 1.1 |
| 444 | 0.83 |
| 445 | 0.91 |
| 446 | 1.05 |
| 447 | 1.1 |
| 448 | 0.89 |
| 449 | 0.95 |
| 450 | 1.05 |
| 451 | 0.97 |
| 452 | 1.13 |
| 453 | 0.92 |
| 454 | 0.93 |
| 455 | 0.97 |
| 456 | 1.49 |
| 457 | 1.4 |
| 458 | 1.07 |
| 459 | 1.13 |
| 460 | 1.03 |
| 461 | 1.19 |
| 462 | 1 |
| 463 | 1.03 |
| 464 | 0.94 |
| 465 | 1 |
| 466 | 1.04 |
| 467 | 1.12 |
| 468 | 1.08 |
| 469 | 1.04 |
| 470 | 1.17 |
| 471 | 0.99 |
| 472 | 1.13 |
| 473 | 0.55 |
| 474 | 0.81 |
| 475 | 0.81 |
| 476 | 0.61 |
| 477 | 0.76 |
| 478 | 0.99 |
| 479 | 1.21 |
| 480 | 0.83 |
| 481 | 0.83 |
| 482 | 0.86 |
| 483 | 0.8 |
| 484 | 0.34 |
| 485 | 0.38 |
| 486 | 0.97 |
| 487 | 0.79 |
| 488 | 0.82 |
| 489 | 0.76 |
| 490 | 0.77 |
| 491 | 0.35 |
| 492 | 0.79 |
| 493 | 0.85 |
| 494 | 0.82 |
| 495 | 0.46 |
| 496 | 0.63 |
| 497 | 0.87 |
| 498 | 0.99 |
| 499 | 0.77 |
| 500 | 0.82 |
| 501 | 0.85 |
| 502 | 0.83 |
| 503 | 0.82 |
| 504 | 0.83 |
| 505 | 0.91 |
| 506 | 0.9 |
| 507 | 0.86 |
| 508 | 0.82 |
| 509 | 0.79 |
| 510 | 0.86 |
| 511 | 0.88 |
| 512 | 0.18 |
| 513 | 1.16 |
| 514 | 0.9 |
| 515 | 0.73 |
| 516 | 0.7 |
| 517 | 0.76 |
| 518 | 1.11 |
| 519 | 1.15 |
| 520 | 1.01 |
| 521 | 0.96 |
| 522 | 1.19 |
| 523 | 1.21 |
| 524 | 1.03 |
| 525 | 1.21 |
| 526 | 0.82 |
| 527 | 0.78 |
| 528 | 0.92 |
| 529 | 0.92 |
| 530 | 1.05 |
| 531 | 1.13 |
| 532 | 1.04 |
| 533 | 0.77 |
| 534 | 1.07 |
| 535 | 1.17 |
| 536 | 1 |
| 537 | 0.99 |
| 538 | 1.4 |
| 539 | 1.12 |
| 540 | 0.96 |
| 541 | 0.87 |
| 542 | 0.85 |
| 543 | 0.99 |
| 544 | 0.96 |
| 545 | 0.84 |
| 546 | 0.89 |
| 547 | 0.95 |
| 548 | 0.83 |
| 549 | 0.85 |
| 550 | 0.85 |
| 551 | 0.81 |
| 552 | 0.82 |
| 553 | 1.33 |
| 554 | 1.04 |
| 555 | 0.61 |
| 556 | 0.79 |
| 557 | 0.87 |
| 558 | 0.88 |
| 559 | 0.81 |
| 560 | 0.93 |
| 561 | 0.85 |
| 562 | 1.14 |
| 563 | 0.94 |
| 564 | 1.04 |
| 564 | 0.9 |
| 566 | 1.51 |
| 567 | 0.52 |
| 568 | 0.61 |
| 569 | 0.97 |
| 570 | 0.87 |
| 571 | 0.6 |
| 572 | 0.37 |
| 573 | 0.5 |
| 574 | 0.7 |
| 575 | 0.31 |
| 576 | 0.85 |
| 577 | 0.86 |
| 578 | 1.25 |
| 579 | 0.31 |
| 580 | 0.86 |
| 581 | 0.86 |
| 582 | 0.76 |
| 583 | 0.78 |
| 584 | 0.85 |
| 585 | 1.17 |
| 586 | 0.76 |
| 587 | 0.75 |
| 588 | 0.86 |
| 589 | 0.91 |
| 590 | 0.82 |
| 591 | 0.79 |
| 592 | 0.78 |
| 593 | 0.72 |
| 594 | 0.84 |
| 595 | 0.74 |
| 596 | 0.79 |
| 597 | 0.82 |
| 598 | 1.23 |
| 599 | 0.84 |
| 600 | 0.81 |
| 601 | 0.78 |
| 602 | 0.79 |
| 603 | 0.61 |
| 604 | 0.69 |
| 605 | 1.01 |
| 606 | 1.24 |
| 607 | 0.78 |
| 608 | 0.79 |
| 609 | 0.78 |
| 610 | 0.86 |
| 611 | 0.71 |
| 612 | 1.15 |
| 613 | 0.9 |
| 614 | 0.89 |
| 615 | 0.76 |
| 616 | 0.64 |
| 617 | 1.06 |
| 618 | 0.77 |
| 619 | 0.78 |
| 620 | 0.74 |
| 621 | 0.78 |
| 622 | 1.06 |
| 623 | 0.8 |
| 624 | 0.79 |
| 625 | 0.87 |
| 626 | 0.81 |

A subset of the ASOs from Table 4 were selected for further validation by ELISA (Example 2) based on reproducibility and a fold-change threshold.

### Example 2

The selected ASOs to STXBP1 from Example 1 were evaluated by ELISA as a separate validation of increased STXBP1 levels.

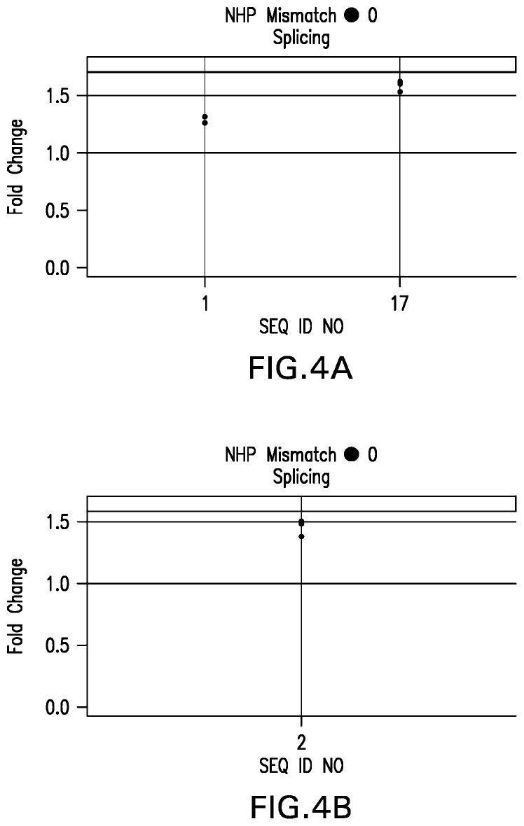

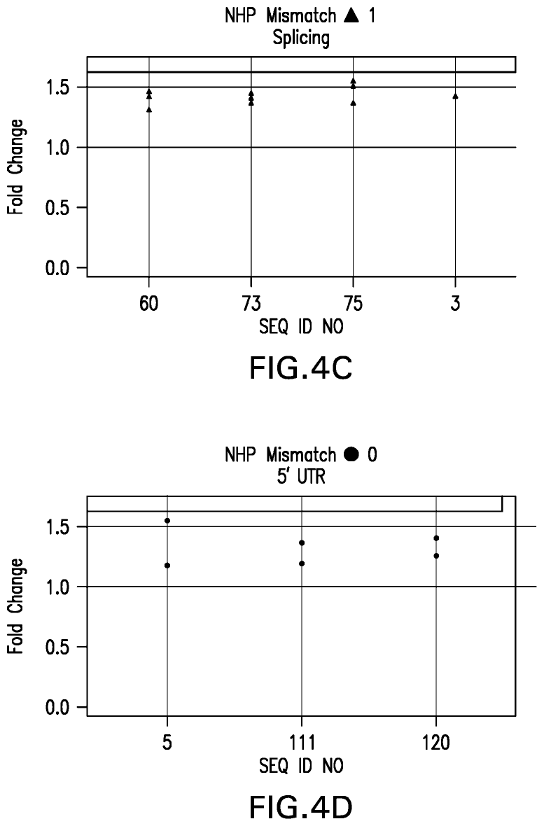

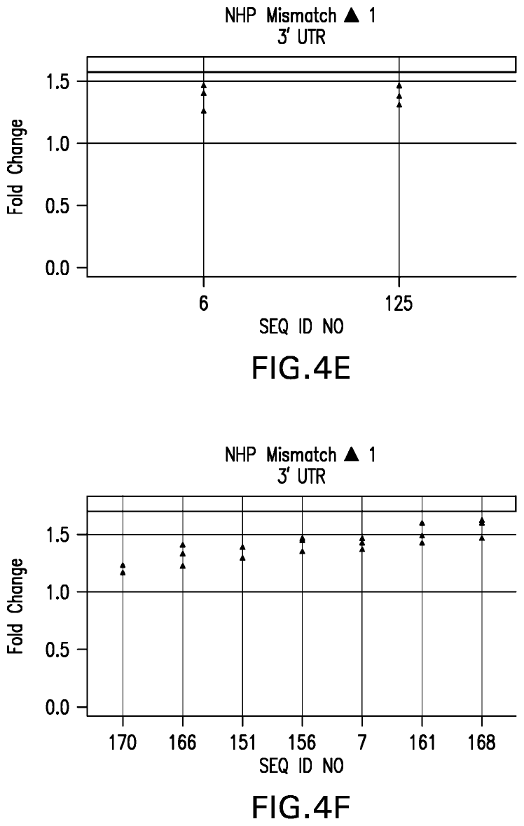

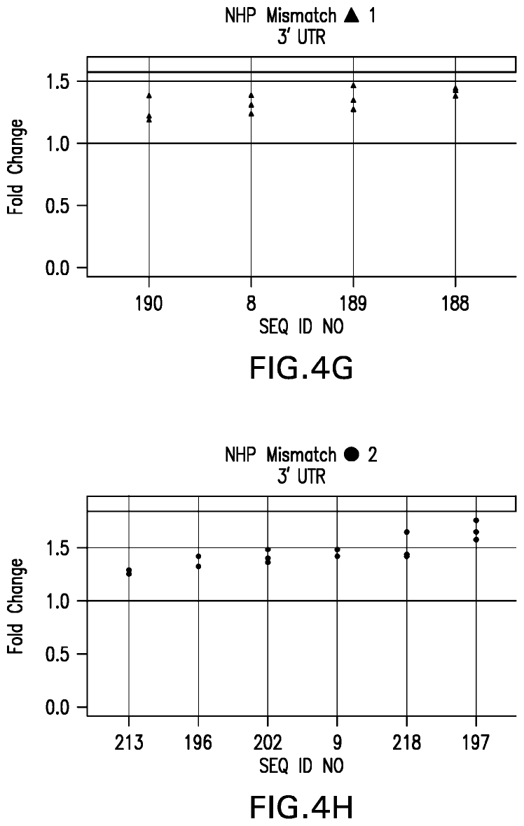

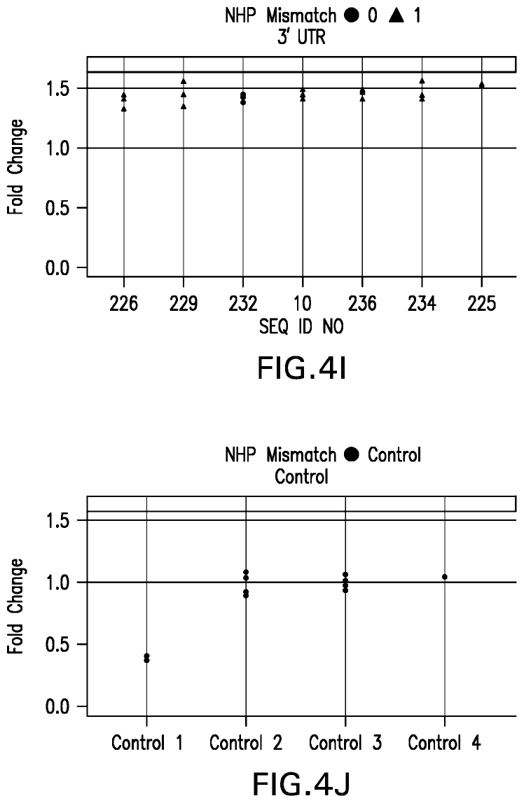

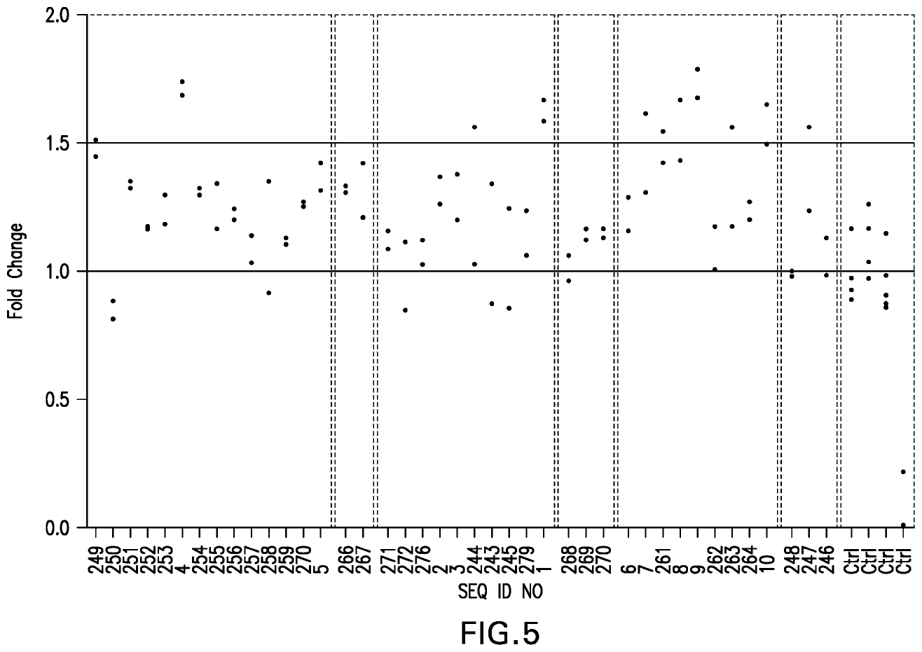

ASOs were screened with ELISA at 5 μM. Briefly, non-HiBiT tagged STXBP1 Het iNeurons were seeded into pre-coated PDL 96-well plates at a density of 50,000 cells/well. On the day after plating, media was removed and ASOs were added at a final concentration of 5 μM. After 17 days, a Cell-Titer Fluor assay was used to estimate the number of viable cells and cells were lysed for ELISA. To avoid artifacts from normalization, concentration values from ELISA were not normalized to the CTF values. Rather, both sets of values were considered individually when evaluating the ASOs. ELISA fold changes were calculated relative to the average of non-targeting controls within the plate. Results are shown in FIGS. 4A-J and 5.

### Example 3

### Dose-Response Study

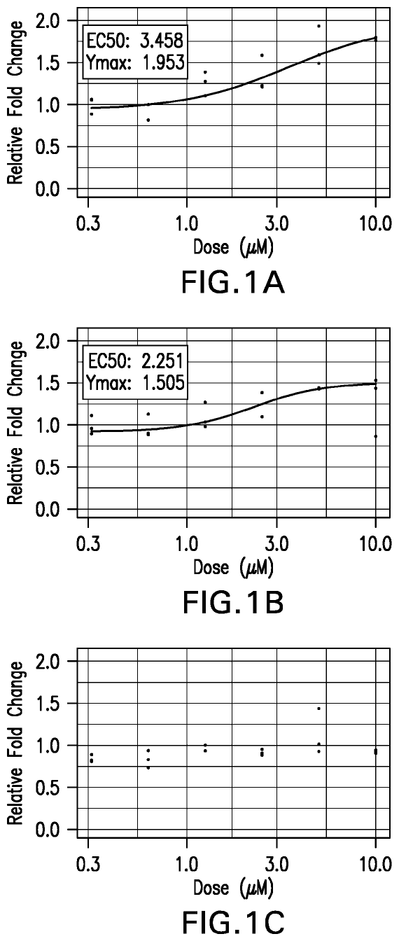

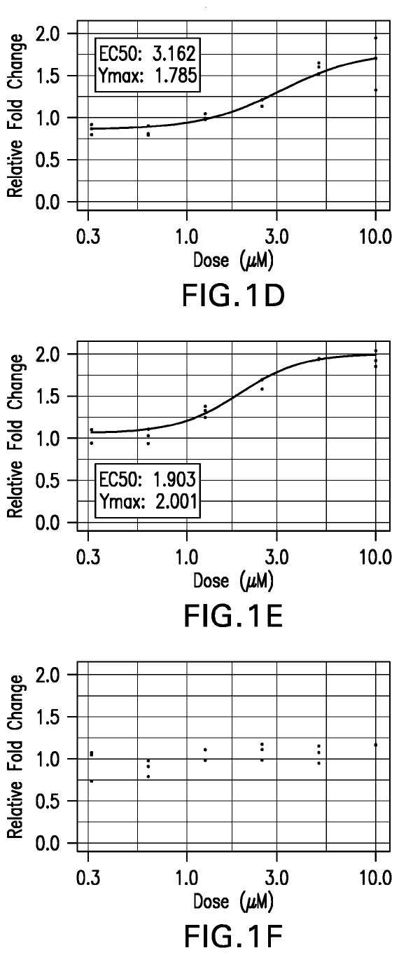

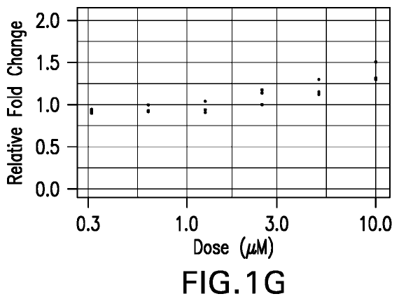

ASOs provided herein were evaluated with a 6-point, 1:2 dilution series dose-response study. Briefly, STXBP1 Het iNeurons were treated by gymnosis with ASOs at 0.3125 μM, 0.625 μM, 1.25 μM, 2.5 μM, 5 μM, and 10 μM. STXBP1 protein levels were measured using the Jess WB system. STXBP1 signals were normalized to a corresponding gamma-tubulin signal as a loading control. Fold change values were calculated relative to non-targeting control at the equivalent dose and displayed in FIGS. 1A-G. Each point represents a technical replicate. Analysis was performed using a median fit to a four-parameter logistic curve. EC₅₀ and Yₘₐₓ, where calculable, are shown in Table 5 below.

TABLE 5
Dose-Response Study shown in EC₅₀ and Yₘₐₓ

| SEQ ID NO: | EC₅₀ (μM) | Yₘₐₓ |
|---|---|---|
| 2 | 3.46 | 1.95 |
| 17 | 2.25 | 1.51 |
| 168 | 3.16 | 1.79 |
| 197 | 1.9 | 2 |

### Example 4

Validation in iNeurons Cell Lines

To test the efficacy of ASOs provided herein, STXBP1 heterozygous (Het) and wild-type (WT) iNeurons were treated in parallel at a 5 μM dose. Data were collected from 2 independent biological replicates, each of which were conducted on different days, used a different batch of neurons and were treated with an independently synthesized lot of ASO compounds. For STXBP1 Het only, data from the 5 μM treatment dose from the dose-response study (Example 3) were included as an additional third biological replicate.

As in the dose-response study described in Example 3, STXBP1 protein levels were measured using the Jess WB system. STXBP1 signals were normalized to a corresponding gamma-tubulin signal as a loading control. Fold change values were calculated relative to the respective STXBP1 Het or WT iNeurons treated with non-targeting control and displayed in the table below.

To test for significant increases in fold change, a one-sample one-sided t-test was used on each ASO against a null hypothesis of 1, where a Fold Change (FC) of 1 implies no change from the non-targeting control μ≤(* p<0.05, ** p<0.01, *** p<0.001). Data are provided below in Table 6.

TABLE 6
Validation in WT iNeurons

| | FC in STXBP1 | FC in |
|---|---|---|
| SEQ ID NO: | Het (5 μM) | WT (5 μM) |
| 2 | 1.53 ± 0.08*** | 1.10 ± 0.09 |
| 75 | 1.07 ± 0.04 | 1.12 ± 0.04* |
| 17 | 1.50 ± 0.07*** | 1.34 ± 0.09** |
| 168 | 1.61 ± 0.08*** | 1.53 ± 0.07*** |
| 188 | 1.21 ± 0.05** | 1.21 ± 0.04** |
| 197 | 1.82 ± 0.08*** | 1.56 ± 0.16** |
| 225 | 1.24 ± 0.03*** | 1.26 ± 0.06** |

### Example 5

### Functional Validation

A MaxWell high-density multi-electrode array (hd-MEA) system was used to evaluate the effect of ASOs provided herein on synaptic function. As neurons mature in vitro, synapse formation leads to synchronized bursts of action potentials, in which the burst frequency is partially regulated by presynaptic function. Sun and Sudhof (2021) Journal of Neuroscience Methods 349, 109041, previously demonstrated with calcium imaging that burst frequency in STXBP1 heterozygous neurons is reduced when compared to WT neurons.

Briefly, STXBP1 Het iNeurons and WT iNeurons were separately co-cultured with human primary astrocytes at a ratio of 5:1 (iNeuron:astrocyte). ASOs were added on Day 1 and Day 15 after plating. Following this protocol, unsynchronized action potential activity can typically be detected approximately two weeks after plating. By ~17-20 days in vitro, action potentials begin to synchronize, however, burst frequency tends to be inconsistent and fluctuates within minutes. To avoid capturing inaccurate burst frequency data during this period, the burst frequency was measured at 28 and 35 days in vitro (DIV) once bursting activity had stabilized. Burst frequency at both time points is displayed in the table below as a percentage of WT. Note that the burst frequency of STXBP1 Het iNeurons treated with non-targeting ASO control at DIV28 is 52.4% of WT, the estimated baseline when evaluating the ASOs provided herein for functional rescue. To test for significant differences at each time point between the treatment conditions and control condition (STXBP1 Het iNeurons treated with non-targeting ASO control), a two-tailed t-test assuming unequal variance was used (* p<0.05, ** p<0.01, ***p<0.001). The data are provided in Table 7 below.

TABLE 7
Synaptic function in STXBP1 Het iNeurons data presented
in (% of WT) MEA Burst Frequency ± Standard Deviation

| | MEA Burst | MEA Burst |
|---|---|---|
| | Frequency (% of | Frequency (% of |
| SEQ ID NO: | WT) at DIV28 | WT) at DIV35 |
| 2 | 77.6 ± 7.4%* | 66.1 ± 7.5% |
| 75 | 54.4 ± 5.0% | 41.2 ± 3.2% |
| 17 | 64.6 ± 4.5% | 58.2 ± 4.4% |
| 168 | 85.7 ± 5.9%** | 81.9 ± 4.6%* |
| 188 | 62.6 ± 4.6% | 53.7 ± 5.4% |
| 197 | 75.5 ± 5.1%* | 70.1 ± 7.0% |
| 225 | 72.8 ± 3.6%* | 67.2 ± 6.9% |
| Non-targeting control | 52.4 ± 5.4% | 57.6 ± 6.0% |

### Example 6

The effect of ASOs on cell viability was evaluated.

### Cell Viability

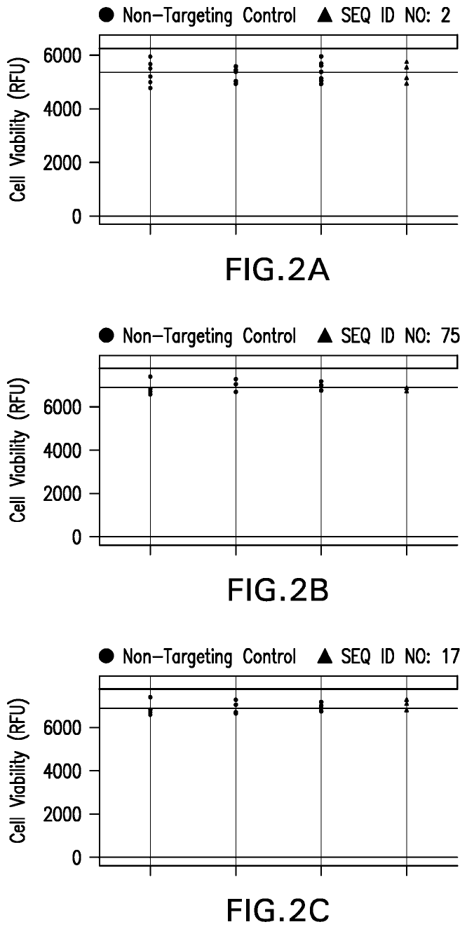

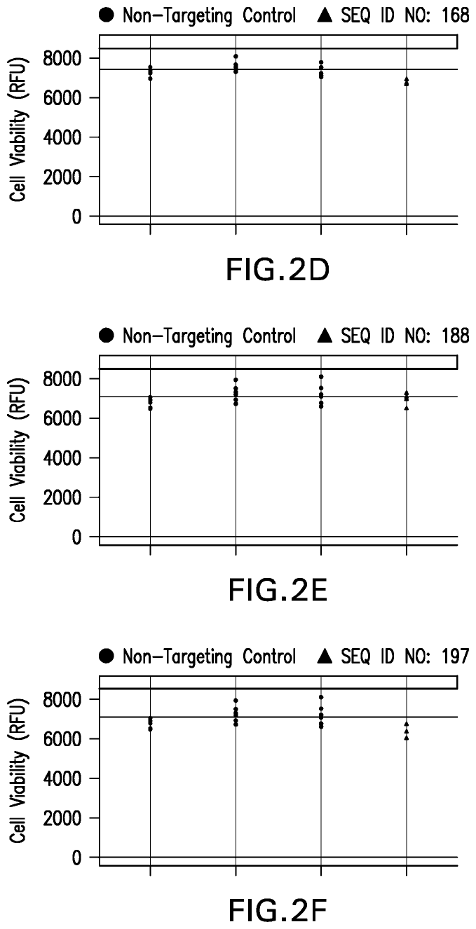

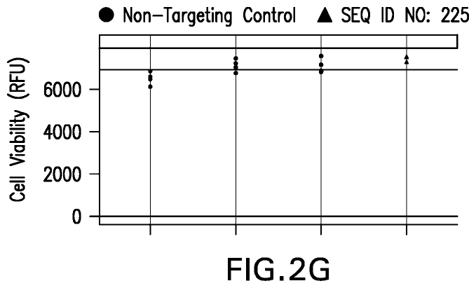

Cell viability was evaluated after a 17-day treatment at a 5 μM dose. Cell viability was determined from using a Cell-Titer Fluor Assay. To facilitate comparisons, raw CTF values (in relative fluorescence units) from 4 technical replicates for each of ASOs were re-plotted in FIGS. 2A-G alongside values from non-targeting ASO controls that were collected from the same plate as the targeted ASO. The cell viability was also calculated as a percentage of the non-targeting ASO controls and displayed in Table 8 below.

To test for significant differences in cell viability between the ASOs provided herein and non-targeting ASO controls, a two-tailed t-test was used assuming unequal variance (* p<0.05, ** p<0.01, *** p<0.001).

TABLE 8
Cell Viability % of viable cells ± Standard Deviation

| | Cell Viability with |
|---|---|
| SEQ ID NO: | 5 μM (% of Ctrl) |
| 2 | 99.51 ± 3.39 |
| 75 | 98.23 ± 0.72 |
| 17 | 102.40 ± 2.09 |
| 168 | 91.37 ± 0.77*** |
| 188 | 98.33 ± 2.36 |
| 197 | 88.80 ± 2.38* |
| 225 | 105.91 ± 1.03** |

### Example 7

### PBMC Study

To test for possible immunostimulatory effects from the ASOs provided herein, a human peripheral blood mononuclear cell (PBMC) study was conducted. Selected ASOs shown in Table 9 below were HPLC-purified (>85% full length), their identity was confirmed by ESI-MS (+/−0.05%) and were endotoxin tested (<0.1 EU/mg). PBMCs were sourced from 7 donors, out of which 5 were selected for the assay. From each of the 5 donors, 100,000 PBMCs were seeded per well (96wp) and ASOs were added for gymnosis at 0.08 μM, 0.4 μM, 2 μM, 10 μM and 40 μM in triplicate. After 24 h, levels of cytokines IL-6, IL-1β, IL-12p70, IFNα2a, IFN-γ and TNF-α were measured using the MSD U-Plex platform.

Internal quality control checks on each of the 5 donors were performed using PBS- and media-only controls in order to assess donor suitability. If any donors were found to consistently exhibit elevated cytokine levels in the absence of any treatment, the results from that donor were considered unsuitable for inclusion in these analyses. For this study, none of the 5 donors used in the PBMC assay were excluded from analyses.

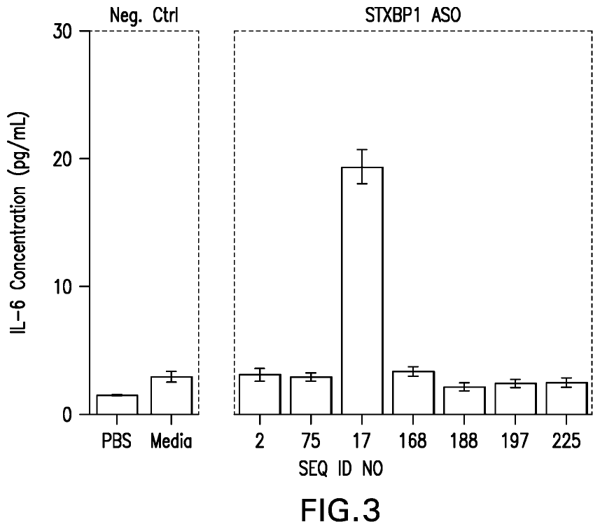

The ASO mediated IL-6 response has been shown to cause immunostimulatory issues in clinical trials. IL-6 results after treatment at 10 μM are displayed in FIG. 3. Error bars represent standard error of the mean. Table 9 below summarizes the mean cytokine concentration values (pg/mL) at 0.4 μM.

TABLE 9
Mean cytokine concentration (pg/mL) at 0.4 μM

| SEQ ID | | | | | | |
|---|---|---|---|---|---|---|
| NO: | IL-6 | IL-1β | IL-12p70 | IFN-α2a | IFN-γ | TNF-α |
| 2 | 3.27 | 0.11 | 0.04 | 0.23 | 0.88 | 1.07 |
| 75 | 4.31 | 0.16 | 0.00 | 0.02 | 1.64 | 0.78 |
| 17 | 5.90 | 0.36 | 0.01 | 0.02 | 0.65 | 0.76 |
| 168 | 2.94 | 0.10 | 0.02 | 0.11 | 0.68 | 0.94 |
| 188 | 2.08 | 0.08 | 0.02 | 0.21 | 0.69 | 0.37 |
| 197 | 2.06 | 0.07 | 0.01 | 0.01 | 0.43 | 0.54 |
| 225 | 2.37 | 0.06 | 0.01 | 0.07 | 0.44 | 0.59 |

### Example 8

In Vitro Efficacy Studies in Human Induced Pluripotent Stem (iPS)-Cell Derived Neuronal and Pathogenic STXBP1 Cell Lines

The ASOs provided herein were tested for STXBP1 RNA modulation, protein increase, synaptic localization, synaptic function, and in vitro network function in neuronal cell cultures. In addition to excitatory neurons that lack one copy of the gene which serve as the baseline, the ASOs provided herein will also be tested in inhibitory neurons and isogenic cell lines engineered with pathogenic patient mutations.

These studies will determine the biological efficacy of the ASO compounds provided herein, in a human patient cell line context.

STXBP1+/−(HZ) Ngn2-Induced Neuron Cultures, Cultured without Glia

STXBP1+/−(CRISPR engineered) Ngn2-induced neuron (BIONi010-C-13), cultured without glia, were treated with vehicle, 5 μM, or 10 μM ASOs, and compared with controls: WT isogenic control cell line+vehicle, HZ+lentivirus overexpressing STXBP1, or HZ+non-targeting control ASO.

Cells were plated at 30 k density, treated with ASOs after 8 and 15 days in vitro, and fixed after 28 days in vitro for immunostaining analysis.

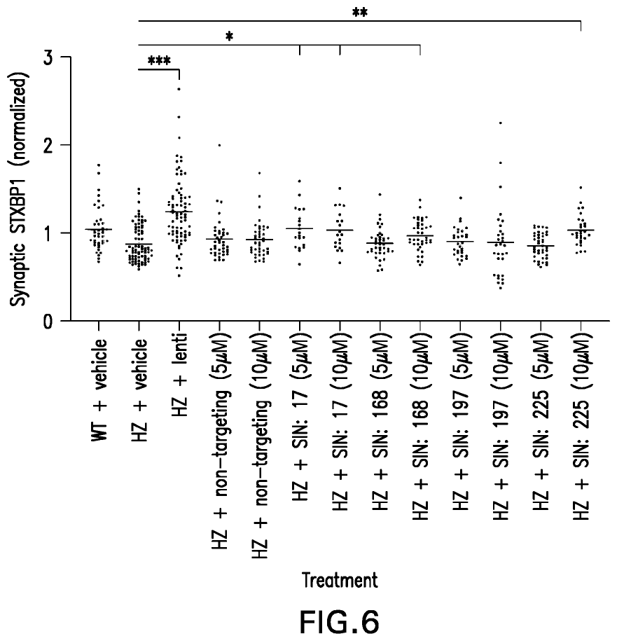

Neuronal cultures were immunostained for STXBP1 (Sigma: HPA023483) and Synaptophysin 1 (Synaptic Systems: SYSY101004). STXBP1 staining co-localized with Synaptophysin 1 was quantified as the primary readout in FIG. 6. Each dot represents a field of view, and fields of view were collected across two independent biological replicates. Horizontal lines represent the median of the distribution.

A key result was that STXBP1+/− neurons treated with ASOs of SEQ ID NOS: 17, 168 and 225 have significantly elevated synaptic STXBP1 expression compared to treatment with vehicle.

Experimental parameters: Cell type: Ngn2-induced neurons with no glia (DIV28); Gymnosis; Treatment: ASOs at 5 or 10 μM; immunostaining: 20 days after ASO treatment
* P≤0.05
** P≤0.01
*** P≤0.001

### WT Ngn2-Induced Neuron Cultures

WT Ngn2-induced neuron (BIONi010-C-13), cultured without glia, were treated with vehicle, 5 μM ASOs, and compared with controls: WT isogenic control cell line+vehicle, WT+lentivirus overexpressing STXBP1, or WT+non-targeting control ASO.

Cells were plated at 30 k density, treated with ASOs after 8 and 15 days in vitro, and fixed after 28 days in vitro for immunostaining analysis.

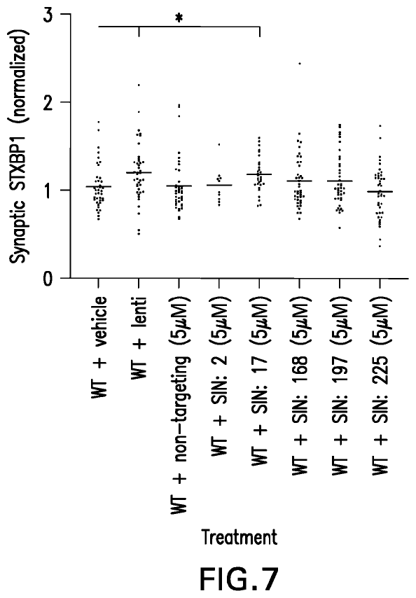

Neuronal cultures were immunostained for STXBP1 (Sigma: HPA023483) and Synaptophysin 1 (Synaptic Systems: SYSY101004). STXBP1 staining co-localized with Synaptophysin 1 was quantified as the primary readout in FIG. 7. Each dot represents a field of view, and fields of view were collected across two independent biological replicates. Horizontal lines represent the median of the distribution.

A key result was that STXBP1 WT neurons treated with an ASO of SEQ ID NO: 17 have significantly elevated synaptic STXBP1 expression compared to treatment with vehicle.

Experimental parameters: Cell type: Ngn2-induced neurons with no glia (DIV28); Gymnosis; Treatment: ASOs at 5 μM; immunostaining: 20 days after ASO treatment
* P≤0.05
   STXBP1+/−(HZ) Ngn2-Induced Neuron Cultures, Cultured with Rat Glia

STXBP1+/−(CRISPR engineered) Ngn2-induced neuron (BIONi010-C-13), cultured with rat glia, were treated with vehicle, 1 μM, 2.5 μM, 5 μM, or 10 μM ASOs, and compared with controls: HZ+lentivirus overexpressing STXBP1, or HZ+non-targeting control ASO.

Cells were plated at 30 k density, treated with ASOs after 8 and 15 days in vitro, and fixed after 28 days in vitro for immunostaining analysis.

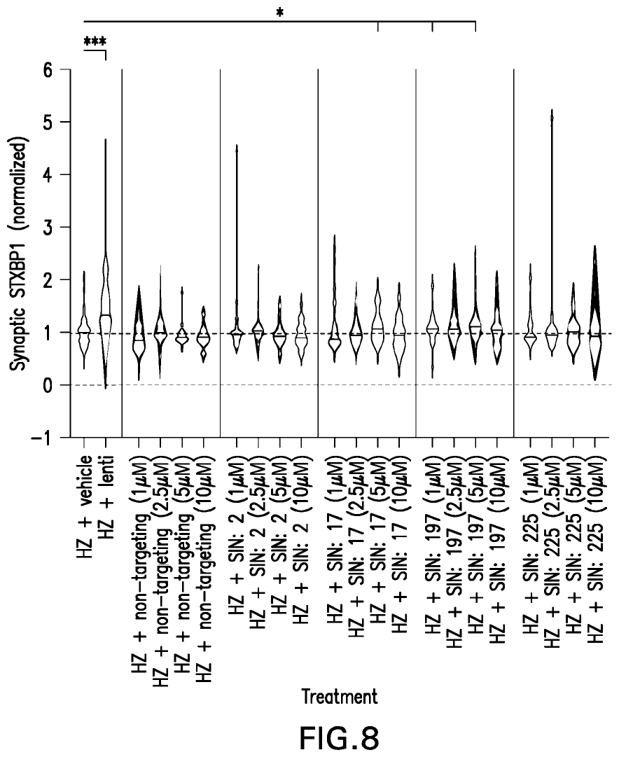

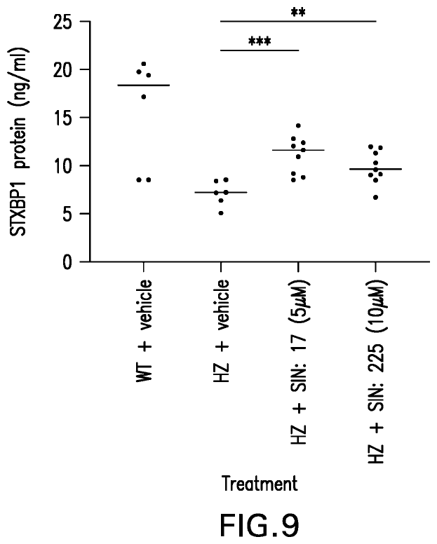

Neuronal cultures were immunostained for STXBP1 (Sigma: HPA023483) and Synaptophysin 1 (Synaptic Systems: SYSY101004). STXBP1 staining co-localized with Synaptophysin 1 was quantified as the primary readout in FIG. 8.

Violin plots represent the distribution of data points (which are individual fields of view collected across two independent biological replicates). Horizontal lines represent the median of the distribution.

A key result was that STXBP1+/− neurons, when cultured with rat glia, and treated with ASOs having SEQ ID NOS: 17 or 197 have significantly elevated synaptic STXBP1 expression compared to treatment with vehicle.

Experimental parameters: Cell type: Ngn2-induced neurons cultured with rat glia (DIV28); Gymnosis; Treatment: ASOs at 5 or 10 μM; immunostaining: 20 days after ASO treatment
* P≤0.05
*** P≤0.001

### Ngn2-Induced Neuron Cultures

STXBP1+/−(CRISPR engineered) Ngn2-induced neuron (BIONi010-C-13), cultured without glia, were treated with vehicle, 5 μM, or 10 μM ASOs, and compared with control: WT isogenic control cell line+vehicle,

Cells were plated at 30 k density, treated with ASOs after 8 days in vitro, and bulk cell lysate was collected after 15 days in vitro for ELISA analysis.

ELISA analysis of bulk STXBP1 protein was performed using a RayBiotech kit (ELH-STXBP1-A, 0621222675), with the calibration curve generated from recombinant human Munc18-1 protein (Abcam: ab267979, GR3337760-2).

Each dot represents a cell culture well (technical replicate), and data was collected across three independent biological replicates. Horizontal lines represent the median of the distribution.

A key result was that STXBP1+/− neurons treated with ASOs having SEQ ID NOS: 17 and 225 have significantly elevated synaptic STXBP1 expression compared to treatment with vehicle.

Experimental parameters: Cell type: Ngn2-induced neurons with no glia (DIV15); Gymnosis; Treatment: ASOs at 5 or 10 μM; ELISA: 7 days after ASO treatment
** P≤0.01
*** P≤0.001

### Example 9

### In Vivo Tolerability and Biodistribution Studies

The tolerability of the ASOs provided herein will be tested by intrathecal injection into adult rats, monitoring for signs of acute in-life toxicity, and brain pathology and biodistribution post-mortem will be analyzed. A parallel mouse intracerebroventricular injection will be performed and long-term toxicity and neurodegeneration will be monitored.

These studies will determine toxicity and biodistribution of the ASO compounds provide herein in a rodent model.

### Example 10

### In Vivo Tolerability and Pharmacodynamics Studies

The cynomolgus monkey will used for testing the tolerability of ASO provided herein administered through intrathecal injection. For ASOs that have perfect homology between human and monkey, we further expect that STXBP1 protein will increase, in vivo. These studies will determine the ASOs tolerability and pharmacodynamics in a wild type monkey.

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

1. An antisense oligonucleotide (ASO) comprising a base sequence that is (i) complementary or reverse complementary to a portion of STXBP1 mRNA or pre-mRNA, or (ii) at least 90% complementary or reverse complementary to the portion of STXBP1 mRNA or pre-mRNA, or (iii) has 1 or 2 mismatches with the portion of STXBP1 mRNA or pre-mRNA or complement thereof.

2. The ASO of claim 1, wherein the STXBP1 pre-mRNA is identical to the coding strand of NCBI Accession No. NC_000009.12 where all thymines are replaced with uracils.

3. The ASO of claim 2 that, when administered to a subject or contacted with a cell, increases expression of STXBP1 protein.

4. The ASO of claim 3, wherein the cell is a neuronal cell and the ASO increases a level of functional STXBP1 protein in the neuronal cell relative to a control neuronal cell not contacted with the ASO.

5. The ASO of claim 3, wherein the base sequence
i) comprises or consists of SEQ ID NO: 17, SEQ ID NO: 168, SEQ ID NO: 197, SEQ ID NO: 225 or any one of SEQ ID NOs: 1-16, 18-167, 169-196, 198-224 or 226-626,
ii) has at least 80%, 85%, 90%, 95% or 100% sequence identity to SEQ ID NO: 17, SEQ ID NO: 168, SEQ ID NO: 197, SEQ ID NO: 225 or any one of SEQ ID Nos: 1-16, 18-167, 169-196, 198-224 or 226-626, or
iii) comprises 10, 11, 12, 13, 14, 15 or 16 consecutive bases of SEQ ID NO: 17, SEQ ID NO: 168, SEQ ID NO: 197, SEQ ID NO: 225 or any one of SEQ ID NOs.: 1-16, 18-167, 169-196, 198-224 or 226-626.

6-7. (canceled)

8. The ASO of claim 5, wherein the base sequence has fewer than 50 nucleotides, optionally that has a length of 10-33 nucleotides.

9. The ASO of claim 5, comprising one or more modified bases, one or more modified sugars, and/or one or more modified internucleoside linkages.

10. The ASO of claim 9 that has at least 50%, 75%, 80%, 85%, 90% or all 2′-MOE sugars and, independently, at least 50%, 75%, 80%, 85%, 90% or all phosphorothioate internucleoside linkages.

11-13. (canceled)

14. An ASO conjugate, comprising (a) the ASO of claim 1, and (b) a heterologous moiety or a targeting moiety.

15. (canceled)

16. The ASO conjugate of claim 14, which is a peptide-ASO conjugate, a PPMO conjugate, or an antibody-ASO conjugate.

17. A pharmaceutical composition, comprising the ASO of claim 1 or an ASO conjugate comprising the ASO of claim 1, and a pharmaceutically acceptable carrier.

18. A method of treating a subject having an STXBP1 disorder or STXBP1 haploinsufficiency, comprising administering to the subject the ASO of claim 1 or an ASO conjugate comprising the ASO of claim 1 or a pharmaceutical composition comprising the ASO or the ASO conjugate.

19. (canceled)

20. The method of claim 18, wherein the STXBP1 disorder is encephalopathy, STXBP1 encephalopathy, epilepsy, epileptic encephalopathy, a severe early onset epileptic encephalopathy, a non-syndromic epilepsy, Ohtahara syndrome, West syndrome, Lennox-Gastaut syndrome, Dravet syndrome, early myoclonic encephalopathy, an unclassified early onset epileptic encephalopathy that is associated with STXBP1 haploinsufficiency, atypical Rett syndrome, a severe intellectual disability without epilepsy associated with STXBP1 haploinsufficiency, global delay, cognitive impairment (mild to profound), a movement disorder, hypotonia, autism, DEE4, Developmental and epileptic encephalopathy 4, Developmental and epileptic encephalopathy, type 4, Early-infantile epileptic encephalopathy 4, EIEE4, STXBP1 encephalopathy with epilepsy, STXBP1 epileptic encephalopathy, STXBP1-related developmental and epileptic encephalopathy, STXBP1-related early-onset encephalopathy, or STXBP1-related epileptic encephalopathy.

21. The method of claim 18 which is a method for treating, preventing or delaying the onset of a disorder caused by an STXBP1 mutation in a subject, comprising administering to the subject the ASO of claim 1 or an ASO conjugate comprising the ASO of claim 1 or a pharmaceutical composition comprising the ASO or ASO conjugate.

22. A method of increasing STXBP1 expression in a cell, comprising contacting the cell with the ASO of claim 1 or an ASO conjugate comprising the ASO of claim 1 or a pharmaceutical composition comprising the ASO or ASO conjugate.

23-24. (canceled)

25. The method of claim 22, wherein the cell is a neuronal cell in vivo.

26. (canceled)

27. The method of claim 22 which is a method of increasing the synaptic local expression of STXBP1 protein in a composition containing neuronal cells, comprising contacting the composition with the ASO of claim 1 or an ASO conjugate comprising the ASO of claim 1 or a pharmaceutical composition comprising the ASO or ASO conjugate.

28. The method of claim 27, wherein the composition containing neuronal cells is in vivo.
